(19) United States
(12) Patent Application Publication
(10) Pub. No.: US 2024/0181049 A1
(43) Pub. Date: Jun. 6, 2024

(54) HARNESSING THE POWER OF MICROBIOTA AND METABOLITES FOR THE TREATMENT OF CANCER

(71) Applicant: UTI LIMITED PARTNERSHIP, CALGARY (CA)

(72) Inventors: Kathleen Dora MCCOY, Calgary (CA); Lukas Franz MAGER, Calgary (CA)

(21) Appl. No.: 17/773,129

(22) PCT Filed: Nov. 2, 2020
(86) PCT No.: PCT/CA2020/051487

§ 371 Date: Apr. 29, 2022

Related U.S. Application Data
(60) Provisional application No. 62/929,340, filed on Nov. 1, 2019.

(51) Int. Cl. A61K 39/395; A61K 31/708; A61K 35/00; A61K 35/741; A61K 35/745; A61K 35/747; A61P 35/00; C12N 1/20; C12R 1/01; C12R 1/225

(52) U.S. Cl. CPC A61K 39/3955; A61K 31/708; A61K 35/741; A61K 35/745; A61K 35/747; A61P 35/00; C12N 1/205; A61K 2035/115; C12R 2001/01; C12R 2001/225

(57) ABSTRACT

Disclosed is a method for treatment of a subject having, or suspected of having, a cancer, in particular colorectal cancer, using an immune checkpoint inhibitor in combination with one or more bacteria selected from Bifidobacterium pseudolongum, Lactobacillus johnsonii, and Olsenella species, wherein the immune checkpoint inhibitor is an anti-CTLA-4 antibody or an anti-PD-1 antibody, wherein antibiotic therapy may precede the use of the immune checkpoint inhibitor and the one or more bacteria.

### RELATED APPLICATION DISCLOSURE

The present application claims the benefit of U.S. Provisional Application Ser. No. 62/929,340 filed Nov. 1, 2019, the disclosure of which is hereby incorporated by reference in its entirety.

### SEQUENCE DISCLOSURE

This application includes as part of its disclosure a Biological Sequence Listing which has been submitted in ASCII format via EFS-Web and is hereby incorporated by reference in its entirety. Said ASCII copy, created on Oct. 30, 2020, is named “1159583o000001.txt” and is 6,261 bytes in size.

### FIELD

The present disclosure relates generally to methods and compositions for treating cancer, and in a specific aspect colorectal cancer. In another aspect the disclosure relates to novel strains of bacteria, as well as compositions and uses thereof.

### BACKGROUND

Colorectal cancer (CRC) is the second and third most common malignancy in Western countries in women and men, respectively (Ferlay et al., 2015). In addition to genetic aberrations, which are essential for the development of CRC, other disease-contributing factors have been identified. These include the microbiota and inflammation, whereby inflammation can drive or inhibit CRC development. Interferon (IFN)-γ producing T helper type 1 (Th1) cells are known to be protective (Mager et al., 2016; Mlecnik et al., 2016; Wang et al., 2015), whereas interleukin (IL)-17-producing Th17 cells promote CRC development (Galon et al., 2006; Grivennikov et al., 2012; Le Gouvello et al., 2008). In fact, the impact of the immune system is so potent that immune cell infiltration in the tumor is a superior prognostic factor compared to the classical tumor-lymph nodes-metastasis (TNM) system in CRC (Anitei et al., 2014; Mlecnik et al., 2016). Similarly, the microbiota also impacts on CRC progression (Arthur et al., 2012; Dejea et al., 2018) and may even alter the efficacy of chemotherapeutics (Iida et al., 2013; Viaud et al., 2013).

Immune checkpoint blockade (ICB) therapy is an efficient anti-cancer strategy that utilizes the therapeutic potential of the immune system. Most notably, ICB inhibitors targeting cytotoxic T-lymphocyte-associated antigen 4 (CTLA-4), programmed cell death protein 1 (PD-1), or its ligand (PD-L1) have shown great success in the treatment of various cancers, including melanoma, renal cell carcinoma, and non-small cell lung cancer (Brahmer et al., 2012; Hodi et al., 2010). More recently, seminal work has shown that the efficacy of ICB therapy is dependent on the presence of certain ICB-promoting gut bacteria (Routy et al., 2018; Sivan et al., 2015; Vetizou et al., 2015).

Despite these exciting advances, ICB therapy efficacy in CRC has been disappointing (Brahmer et al., 2012), with only 5-10% of all CRC patients responding (Le et al., 2017). Moreover, the detailed molecular mechanisms through which bacteria enhance the efficacy of ICB therapies remains unclear. Here, we identified three bacterial species that promote ICB efficacy in CRC and identified inosine as a critical bacterial metabolite that promoted differentiation of Th1-mediated anti-tumor immunity.

### SUMMARY

In one aspect there is provided a method of treating a subject having a cancer or suspected of having a cancer, comprising or consisting of, administering an immune checkpoint inhibitor and one or more bacterium selected from Bifidobacterium pseudolongum, Lactobacillus johnsonii, Olsenella profuse, Olsenella umbonata, or Olsenella uli or a combination thereof.

In one aspect there is provided a method of treating a subject having a cancer or suspected of having a cancer, comprising or consisting of, administering an immune checkpoint inhibitor and one or more bacterium selected from Bifidobacterium pseudolongum, Lactobacillus johnsonii, or Olsenella sp. or a combination thereof.

In an exemplary embodiment the bacterium is selected from the Bifidobacterium pseudolongum strain deposited as IDAC Deposit No. 231020-01, Lactobacillus johnsonii strain deposited as IDAC Deposit No. 231020-02, or Olsenella sp. strain deposited as IDAC Deposit No. 231020-03, or a combination thereof.

In one aspect there is provided a method of treating a subject having or suspected of having colorectal cancer (CRC), comprising or consisting of, administering an immune checkpoint inhibitor and one or more bacterium selected from Bifidobacterium pseudolongum, Lactobacillus johnsonii, Olsenella profuse, Olsenella umbonata, or Olsenella uli, such as the Bifidobacterium pseudolongum strain deposited as IDAC Deposit No. 231020-01, the Lactobacillus johnsonii strain deposited as IDAC Deposit No. 231020-02, or the Olsenella sp. strain deposited as IDAC Deposit No. 231020-03, or a combination thereof.

In one aspect there is provided a method of treating a subject having or suspected of having colorectal cancer (CRC), comprising or consisting of, administering an immune checkpoint inhibitor and one or more bacteria selected from Bifidobacterium pseudolongum (B.p.), Lactobacillus johnsonii (L.j), or Olsenella sp. (O.sp.), such as the Bifidobacterium pseudolongum strain deposited as IDAC Deposit No. 231020-01, the Lactobacillus johnsonii strain deposited as IDAC Deposit No. 231020-02, or the Olsenella sp. strain deposited as IDAC Deposit No. 231020-03, or a combination thereof.

In one aspect there is provided a method of treating a subject having or suspected of having colorectal cancer (CRC), comprising or consisting of, administering an immune checkpoint inhibitor and one or more bacteria selected from Bifidobacterium sp. (B.sp.), Lactobacillus sp. (L.sp.), or Olsenella sp. (O.sp.), such as the Bifidobacterium pseudolongum strain deposited as IDAC Deposit No. 231020-01, the Lactobacillus johnsonii strain deposited as IDAC Deposit No. 231020-02, or the Olsenella sp. strain deposited as IDAC Deposit No. 231020-03, or a combination thereof.

In one aspect there is provided a use of an immune checkpoint inhibitor and one or more bacterium selected from Bifidobacterium pseudolongum, Lactobacillus johnsonii, Olsenella profuse, Olsenella umbonata, or Olsenella uli, or a combination thereof, for treating a subject having a cancer or suspected of having a cancer. Said bacterium may comprise Bifidobacterium pseudolongum strain deposited as IDAC Deposit No. 231020-01, the Lactobacillus johnsonii strain deposited as IDAC Deposit No. 231020-02, or the Olsenella sp. strain deposited as IDAC Deposit No. 231020-03, or a combination thereof.

In one aspect there is provided a use of an immune checkpoint inhibitor and one or more bacterium selected from Bifidobacterium pseudolongum, Lactobacillus johnsonii, or Olsenella sp., or a combination thereof, for treating a subject having a cancer or suspected of having a cancer. Said bacterium may comprise Bifidobacterium pseudolongum strain deposited as IDAC Deposit No. 231020-01, the Lactobacillus johnsonii strain deposited as IDAC Deposit No. 231020-02, or the Olsenella sp. strain deposited as IDAC Deposit No. 231020-03, or a combination thereof.

In one aspect there is provided a use of an immune checkpoint inhibitor and one or more bacterium selected from Bifidobacterium pseudolongum, Lactobacillus johnsonii, Olsenella profuse, Olsenella umbonata, or Olsenella uli, or a combination thereof, for treating a subject having or suspected of having colorectal cancer (CRC). Said bacterium may comprise Bifidobacterium pseudolongum strain deposited as IDAC Deposit No. 231020-01, the Lactobacillus johnsonii strain deposited as IDAC Deposit No. 231020-02, or the Olsenella sp. strain deposited as IDAC Deposit No. 231020-03, or a combination thereof.

In one aspect there is provided a use of an immune checkpoint inhibitor and one or more bacteria selected from Bifidobacterium pseudolongum (B.p.), Lactobacillus johnsonii (L.j), or Olsenella sp. (O.sp.), or a combination thereof, for treating a subject having or suspected of having colorectal cancer (CRC). Said bacterium may comprise Bifidobacterium pseudolongum strain deposited as IDAC Deposit No. 231020-01, the Lactobacillus johnsonii strain deposited as IDAC Deposit No. 231020-02, or the Olsenella sp. strain deposited as IDAC Deposit No. 231020-03, or a combination thereof.

In one aspect there is provided a use of an immune checkpoint inhibitor and one or more bacteria selected from Bifidobacterium sp. (B.sp.), Lactobacillus sp. (L.sp.), or Olsenella sp. (O.sp.), or a combination thereof, for treating a subject having or suspected of having colorectal cancer (CRC). Said bacterium may comprise Bifidobacterium pseudolongum strain deposited as IDAC Deposit No. 231020-01, the Lactobacillus johnsonii strain deposited as IDAC Deposit No. 231020-02, or the Olsenella sp. strain deposited as IDAC Deposit No. 231020-03, or a combination thereof.

In one aspect there is provided a kit for treating a subject having a cancer or suspected of having a cancer, comprising or consisting of, an immune checkpoint inhibitor and one or more bacterium selected from Bifidobacterium pseudolongum, Lactobacillus johnsonii, Olsenella profuse, Olsenella umbonata, or Olsenella uli, or a combination thereof and optionally a container. In an exemplary embodiment the bacterium is selected from the Bifidobacterium pseudolongum strain deposited as IDAC Deposit No. 231020-01, Lactobacillus johnsonii strain deposited as IDAC Deposit No. 231020-02, or Olsenella sp. strain deposited as IDAC Deposit No. 231020-03, or a combination thereof.

In one aspect there is provided a kit for treating a subject having a cancer or suspected of having a cancer, comprising or consisting of an immune checkpoint inhibitor and one or more bacterium selected from Bifidobacterium pseudolongum, Lactobacillus johnsonii, or Olsenella sp., or a combination thereof, and optionally a container. In an exemplary embodiment the bacterium is selected from the Bifidobacterium pseudolongum strain deposited as IDAC Deposit No. 231020-01, Lactobacillus johnsonii strain deposited as IDAC Deposit No. 231020-02, or Olsenella sp. strain deposited as IDAC Deposit No. 231020-03, or a combination thereof.

In one aspect there is provided a kit for treating a subject having or suspected of having colorectal cancer (CRC), comprising or consisting of, an immune checkpoint inhibitor and one or more bacterium selected from Bifidobacterium pseudolongum, Lactobacillus johnsonii, Olsenella profuse, Olsenella umbonata, or Olsenella uli or a combination thereof and optionally a container. In an exemplary embodiment the bacterium is selected from the Bifidobacterium pseudolongum strain deposited as IDAC Deposit No. 231020-01, Lactobacillus johnsonii strain deposited as IDAC Deposit No. 231020-02, or Olsenella sp. strain deposited as IDAC Deposit No. 231020-03, or a combination thereof.

In one aspect there is provided a kit for treating a subject having or suspected of having colorectal cancer (CRC), comprising or consisting of, administering an immune checkpoint inhibitor and one or more bacteria selected from Bifidobacterium pseudolongum (B.p.), Lactobacillus johnsonii (L.j), or Olsenella sp. (O.sp.) and optionally a container. In an exemplary embodiment the bacterium is selected from the Bifidobacterium pseudolongum strain deposited as IDAC Deposit No. 231020-01, Lactobacillus johnsonii strain deposited as IDAC Deposit No. 231020-02, or Olsenella sp. strain deposited as IDAC Deposit No. 231020-03, or a combination thereof.

In one aspect there is provided a kit for treating a subject having or suspected of having colorectal cancer (CRC), comprising or consisting of, administering an immune checkpoint inhibitor and one or more bacteria selected from Bifidobacterium sp. (B.sp.), Lactobacillus sp. (L.sp.), or Olsenella sp. (O.sp.) and optionally a container. In an exemplary embodiment the bacterium is selected from the Bifidobacterium pseudolongum strain deposited as IDAC Deposit No. 231020-01, Lactobacillus johnsonii strain deposited as IDAC Deposit No. 231020-02, or Olsenella sp. strain deposited as IDAC Deposit No. 231020-03, or a combination thereof.

In one aspect there is provided a method of treating a subject having a cancer or suspected of having a cancer, comprising or consisting of, administering: an immune checkpoint inhibitor; inosine, a derivative of inosine, functional derivative of inosine, a prodrug of inosine, or a physiologically functional derivative of inosine; and a co-stimulant.

In one aspect there is provided a method of treating a subject having or suspected of having colorectal cancer (CRC), comprising or consisting of, administering: an immune checkpoint inhibitor; inosine, a derivative of inosine, functional derivative of inosine, a prodrug of inosine, or a physiologically functional derivative of inosine; and a co-stimulant.

In one aspect there is provided a use of an immune checkpoint inhibitor; inosine, a derivative of inosine, functional derivative of inosine, a prodrug of inosine, or a physiologically functional derivative of inosine; and a co-stimulant, for treating a subject having a cancer or suspected of having a cancer.

In one aspect there is provided a use of an immune checkpoint inhibitor; inosine, a derivative of inosine, functional derivative of inosine, a prodrug of inosine, or a physiologically functional derivative of inosine; and a co-stimulant, for treating a subject having a cancer or suspected of having a cancer.

In one aspect there is provided a kit for treating a subject having a cancer or suspected of having a cancer, comprising or consisting of an immune checkpoint inhibitor; inosine, a derivative of inosine, functional derivative of inosine, a prodrug of inosine, or a physiologically functional derivative of inosine; and a co-stimulant, and optionally a container.

In one example, the cancer is colorectal cancer (CRC), lung cancer, melanoma, bladder cancer, kidney cancer, breast cancer, prostate cancer, stomach cancer, liver cancer, esophageal cancer, pancreatic cancer, brain cancer, cervical cancer, ovarian cancer, thyroid cancer, lip cancer, oral cancer, larynx cancer, nasopharynx cancer, or uterine cancer.

In an exemplary embodiment the cancer is a solid cancer. In an exemplary embodiment the cancer is a blood cancer (e.g., a leukemia or a lymphoma).

In another example, the cancer is selected from non-small cell lung cancer, small cell lung cancer, gastric carcinoma, testicular cancer, mesothelioma, head and neck cancers, glioblastoma, thymic carcinoma, or Merkel cell cancer. In another example, the cancer is selected from leukemias, myeloproliferative neoplasms (MPN), myelodysplastic syndromes (MDS), chronic lymphocytic leukemia (CLL), chronic myelocytic leukemia (CML), acute lymphoblastic leukemia (ALL), acute myeloid leukemia (ALL), myelodysplastic syndrome (MDS), Hodgkin lymphoma (HL), Non-Hodgkin lymphoma (NHL), multiple myeloma (MM), polycythemia vera (PV), essential thrombocythemia (ET), primary myelofibrosis (PMF), chronic eosinophilic leukemia, or mycosis fungoides.

In one example the cancer is mismatch repair deficient, such as an MMRD colorectal cancer, gastrointestinal cancer, endometrial cancer, breast cancer, prostate cancer, bladder cancer, or thyroid cancer, and/or in a subject having Lynch syndrome. In one example, the cancer is a CRC that is mismatch repair deficient (MMRD) CRC or inflammation-associated CRC. In exemplary embodiments the MMRD is determined based on a lack of functional expression of one or more mismatch repair proteins, e.g., MLH1, MSH2, MSH6 and PMS2 gene. MMRD may result from a loss of function in or decreased expression of at least one of mismatch repair protein, such as due to gene methylation, e.g., in the MLH1 gene. MMRD deficiency can be determined by immunohistochemical analysis of mismatch repair proteins. Said MMRD may be determined based on cancer histological features, e.g., increased tumor infiltrating lymphocytes, medullary or micro-glandular morphology, and/or mucinous or signet ring cell morphology in 50% or more of the tumor. MMRD may also be identified by the presence of microsatellite instability (MSI).

In one example, said ICB inhibitor is an anti-CTLA4 antibody, or an anti-PD-L1 antibody, or an anti-PD-1 antibody.

In one example said ICB inhibitor is an antagonist of CTLA-4, PD-1, PD-L1, PD-L2, LAG-3, VISTA, IDO, IDO1 IDO2, TIGIT, BTLA, HVEM, CD226 (DNAM-1), CD96 (Tactile), TIM-3, LAIR1, CD160 (BY55), CD244 (2B4), VTCN1 (B7-H4), KIR, A2_{A}R, or B7-H3.

In one example said ICB inhibitor is a small molecule antagonist of CTLA-4, PD-1, PD-L1, PD-L2, LAG-3, VISTA, IDO, IDO1 IDO2, TIGIT, BTLA, HVEM, CD226 (DNAM-1), CD96 (Tactile), TIM-3, LAIR1, CD160 (BY55), CD244 (2B4), VTCN1 (B7-H4), KIR, A2_{A}R, or B7-H3.

In one example said ICB inhibitor comprises an antagonist antibody that specifically binds to CTLA-4, PD-1, PD-L1, PD-L2, LAG-3, VISTA, IDO, IDO1 IDO2, TIGIT, BTLA, HVEM, CD226 (DNAM-1), CD96 (Tactile), TIM-3, LAIR1, CD160 (BY55), CD244 (2B4), VTCN1 (B7-H4), KIR, A2_{A}R, or B7-H3.

In one example said ICB inhibitor comprises a fragment of CTLA-4, PD-1, PD-L1, PD-L2, LAG-3, VISTA, IDO, IDO1 IDO2, TIGIT, BTLA, HVEM, CD226 (DNAM-1), CD96 (Tactile), TIM-3, LAIR1, CD160 (BY55), CD244 (2B4), VTCN1 (B7-H4), KIR, A2_{A}R, or B7-H3, or comprises a fragment of a binding partner (e.g., receptor or ligand) of any of the foregoing.

In exemplary embodiments, said ICB inhibitor comprises an antibody, small molecule, or fusion protein, or a combination thereof. In exemplary embodiments, said ICB inhibitor is selected from ipilimumab (YERVOY®, anti-CDLA-4 antibody, Bristol-Myers Squibb), nivolumab (OPDIVO®, anti-PD-1 antibody, Bristol-Myers Squibb), pembrolizumab (KEYTRUDA®, anti-PD-1 antibody, Merck), atezolizumab (TECENTRIQ®, anti-PD-L1 antibody, Roche), avelumab (BAVENCIO®, anti-PD-L1 antibody, Merck KGaA/Pfizer), durvalumab (IMFINZI®, anti-PD-L1 antibody, Medimmune/AstraZeneca), cemiplimab (LIBTAYOR, anti-PD-1 antibody, Regeneron/Sanofi), lambrolizumab (anti-PD-1 antibody, Merck), pidilizumab (anti-PD-1 and anti-DLL antibody, Medivation), BMS-936559 (anti-PD-L1, Bristol-Myers Squibb), MEDI-0680 (anti-PD-1 antibody; AMP-514; AstraZeneca), REGN2810 (anti-PD-1 antibody, Regeneron), CA-170 (small molecule PD-1 and PD-L1 inhibitor; Curis), BMS-1166 (small molecule PD-L1 inhibitor, Bristol-Myers Squibb), AMP-224 (anti-PD-1 fusion protein, Medimmune), spartalizumab (anti-PD-1 antibody, Novartis), STI-A1110 (anti-PD1 antibody, Sorrento/Servier), Dostarlimab (anti-PD-1 antibody, TSR-042, Tesaro), RG-7446 (anti-PD-L1 antibody, Roche), AUR-012 (peptide antagonist of PD1, Aurigene), STI-A1010 (anti-PD-L1 antibody, Sorrento), or a combination thereof.

In one example, the Bifidobacterium sp. is presented in FIG. 22.

In one example, the Lactobacillus sp. is presented in FIG. 23.

In one example, the Olsenella sp. is presented in FIG. 24.

In one example, the Bifidobacterium sp. comprises a 16S rDNA sequence having at least 85%, such as at least 90%, at least 95%, at least 96%, at least 97%, at least 98%, at least 99%, at least 99.5%, or having 100% identity to SEQ ID NO: 1.

In one example, the Lactobacillus sp. comprises a 16S rDNA sequence having at least 85%, such as at least 90%, at least 95%, at least 96%, at least 97%, at least 98%, at least 99%, at least 99.5%, or having 100% identity to SEQ ID NO: 2.

In one example, the Olsenella sp. comprises a 16S rDNA sequence having at least 85%, such as at least 90%, at least 95%, at least 96%, at least 97%, at least 98%, at least 99%, at least 99.5%, or having 100% identity to SEQ ID NO: 3.

In one example, the method or use or kit or use of a kit further comprises administration of a chemotherapeutic agent, an immunotherapeutic agent, or a radiotherapy, or a combination thereof.

In one example, said subject is a human. Said human subject may be of any age, e.g., infant, child, adolescent, adult, or elderly.

In one example, said subject is a non-human animal, such as a non-human primate, a companion animal (e.g., a mammalian animal such as a dog, cat, ferret, horse, rabbit, guinea pig, gerbil, hamster, chinchilla, rat, mouse, or other small mammal; a bird; a reptile; a fish; an amphibian; an arthropod) or a livestock animal (e.g., a mammalian livestock animal such as a cow, pig, sheep, goat, alpaca, donkey, camel, water buffalo, or mink; or a chicken).

In exemplary embodiments, said bacteria may be a strain that raises the level of inosine, xanthine, hypoxanthine and/or xanthine monophosphate, preferably inosine or hypoxanthine, in vivo or in an in vitro secretion assay.

In exemplary embodiments, said bacteria may be administered in an effective amount to raise the level of inosine, xanthine, hypoxanthine and/or xanthine monophosphate in said subject.

In exemplary embodiments, said bacteria may be administered in an effective amount to sensitize said cancer to treatment with said immune checkpoint inhibitor.

In one example, the CRC is mismatch repair deficient (MMRD) CRC or inflammation-associated CRC. In exemplary embodiments the MMRD is determined based on a lack of functional expression of one or more mismatch repair proteins, e.g., MLH1, MSH2, MSH6 and PMS2 gene. MMRD may result from a loss of function in or decreased expression of at least one of mismatch repair protein, such as due to gene methylation, e.g., in the MLH1 gene. MMRD deficiency can be determined by immunohistochemical analysis of mismatch repair proteins. Said MMRD may be determined based on cancer histological features, e.g., increased tumor infiltrating lymphocytes, medullary or micro-glandular morphology, and/or mucinous or signet ring cell morphology in 50% or more of the tumor. MMRD may also be identified by the presence of microsatellite instability (MSI).

In one example, said co-stimulant is Toll like receptor (TLR) signals, CpG, LPS, Flagellin, Nucleotide-binding oligomerization domain-like receptors (NLRs), meso-diaminopimelic acid, muramyl dipeptide, ATP, extracellular glucose, crystals of monosodium urate, calcium pyrophosphate dihydrate, alum, cholesterol or environmental irritants; silica; asbestos; UV irradiation and skin irritants. RIG-I-like receptors (retinoic acid-inducible gene-I-like receptors), single- or double-stranded RNA (e.g., from viruses), C-type lectin receptors (CLR), repeated mannose units, C-type lectin domain, Cytokine receptor signalling, IL-12, IL-18, IL-33, IFN-g, Stimulation provided through antigen presenting cells or their counterpart on T-cells, CD80-CD28, CD86-CD28, CD40CD40L, OX-40L-OX40, -cGAS-STING pathway, for example, cytosolic DNA.

In another aspect, the disclosure provides an isolated bacterium comprising a 16S rDNA sequence having at least 85%, at least 90%, at least 95%, at least 96%, at least 97%, at least 98%, at least 99%, at least 99.5%, or having 100% identity to SEQ ID NO: 1, preferably having at least 99.5%, or having 100% identity to SEQ ID NO: 1.

In another aspect, the disclosure provides an isolated bacterium comprising a 16S rDNA sequence having at least 85%, at least 90%, at least 95%, at least 96%, at least 97%, at least 98%, at least 99%, at least 99.5%, or having 100% identity to SEQ ID NO: 2, preferably having at least 99.5%, or having 100% identity to SEQ ID NO: 2.

In another aspect, the disclosure provides an isolated bacterium comprising a 16S rDNA sequence having at least 85%, such as at least 90%, at least 95%, at least 96%, at least 97%, at least 98%, at least 99%, at least 99.5%, or having 100% identity to SEQ ID NO: 3, preferably having at least 95%, at least 96%, at least 97%, at least 98%, at least 99%, at least 99.5%, or having 100% identity to SEQ ID NO: 3.

In another aspect, the disclosure provides an isolated bacterium of the Bifidobacterium pseudolongum strain deposited as IDAC Deposit No. 231020-01.

In another aspect, the disclosure provides an isolated bacterium of the Lactobacillus johnsonii strain deposited as IDAC Deposit No. 231020-02.

In another aspect, the disclosure provides an isolated bacterium of the Olsenella sp. strain deposited as IDAC Deposit No. 231020-03.

In another aspect, the disclosure provides a composition comprising a bacterium of any of the aforementioned bacteria and a pharmaceutically acceptable carrier.

In another aspect, the disclosure provides a composition comprising an effective amount of any of the aforementioned bacteria for the treatment of a cancer and optionally further comprising a pharmaceutically acceptable carrier.

In another aspect, the disclosure provides a composition comprising a mixture of two or more of the aforementioned strains of bacteria and optionally further comprising a pharmaceutically acceptable carrier.

In another aspect, the disclosure provides a composition comprising an effective amount of a mixture of two or more of the aforementioned strains of bacteria for the treatment of a cancer and optionally further comprising a pharmaceutically acceptable carrier.

In another aspect, the disclosure provides a food, beverage, food supplement, probiotic, or nutraceutical comprising a bacterium of any of the aforementioned bacteria, which preferably is formulated for ingestion.

In exemplary embodiments, said bacteria produce elevated levels of inosine, xanthine, hypoxanthine, and/or inosine monophosphate, preferably inosine, in an in vitro or in vivo assay.

In exemplary embodiments, said bacterium or composition is lyophilized.

In exemplary embodiments, said bacterium or composition is adapted for administration to a subject, preferably a human subject. Said human subject may be of any age, e.g., infant, child, adolescent, adult, or elderly. Said subject may be a non-human animal, such as a non-human primate, a companion animal (e.g., a mammalian animal such as a dog, cat, ferret, horse, rabbit, guinea pig, gerbil, hamster, chinchilla, rat, mouse, or other small mammal; a bird; a reptile; a fish; an amphibian; an arthropod) or a livestock animal (e.g., a mammalian livestock animal such as a cow, pig, sheep, goat, alpaca, donkey, camel, water buffalo, or mink; or a chicken).

In exemplary embodiments, said bacterium or composition is adapted for use in any of the methods disclosed herein, e.g., methods of treating cancer as described above.

In exemplary embodiments, said bacterium or composition contains an effective amount of said bacteria for treating a subject having a cancer or suspected of having a cancer according to the method disclosed herein.

### BRIEF DESCRIPTION OF SEVERAL VIEWS OF THE DRAWINGS

Embodiments of the present disclosure will now be described, by way of example only, with reference to the attached Figures.

FIG. 1A-1J: Immune cell and microbial dynamics upon ICB therapy in AOM/DSS tumors. (a) Overview of the experimental setup of AOM/DSS-induced CRC and ICB treatment. (b) Tumor weight, (c) number of tumors, (d) Ep-CAM⁺LGR5⁺ cancer stem cells and (e) tumor-infiltrating leukocytes (TILs) 140 days post induction in animals treated with isotype-, anti-PD-L1 or anti-CTLA-4 antibodies. (f) CD8⁺ T cell frequencies in the tumor draining lymph node at day 140. Splenic IFN-γ⁺ production in (g) CD4⁺ or (h) CD8⁺ T cells. (i) 16S rRNA gene V4 region amplicon sequencing to identify bacteria in tumor tissue. Bacteria enriched or reduced in tumors of anti-PD-L1/anti-CTLA-4 compared to isotype treated animals are shown in green or red, respectively. (j) Bacteria cultured from homogenized tumors under anaerobe conditions from anti-PD-L1/anti-CTLA-4 (ICB groups) or isotype (Isotype group) treated animals. Bacteria depicted as green or red could only be cultured in ICB groups or Isotype group, respectively. Bacteria depicted as brown were present in both groups. Data are (b-h) mean±SEM or (i) mean #lfcSE (logfoldchangeStandard Error) and pooled from three individual experiments. (b-f) n=16-20 mice/group, (g and h) n=4-5 mice/group. *, P<0.05; **, P<0.01; ***, P<0.001; ****, P<0.0001.

FIG. 2A-21: Individual bacterial species boost ICB therapy. (a) Schematic of the experimental setup to assess the effect of individual bacteria on anti-CTLA-4 therapy efficacy. (b) Tumor growth, (c) tumor weight, (d) live tumor cells and (e) representative pictures of tumors are shown at day 18. Scale bars: 1 cm. (f) representative plots and (g) quantification of IFN-γ⁺ and Ki-67⁺CD8⁺ T cells at day 18 in the tumor tissue. (h and i) same as (f and g), but for CD4⁺ T cells. Data are mean±SEM and pooled from three individual experiments (n=8-15 mice/group). *, P<0.05; **, P<0.01; ***, P<0.001; ****, P<0.0001.

FIG. 3A-3N: Effect of B.p., anti-CTLA-4 and B.p. conditioned serum on T cell differentiation and activation. (a, h and k) Schematic of the experimental setups. (b) representative plots and (c) quantification of T-bet⁺ and T-bet⁺IFN-γ⁺ events of CD3ε⁺CD4⁺ cells in the small intestine (SI) in the presence of indicated bacteria at day 28. (d and e) same as b and c, but in the mesenteric lymph node (MLN). (f and g) same as b and c, but in the spleen. (i) representative plots and (j) quantification of T-bet⁺ and T-bet⁺IFN-γ⁺ events of CD3ε⁺CD4⁺ T cells in the spleen in the presence of indicated bacteria and anti-CTLA-4 treatment at day 32. (l) Tumor growth and weight are shown 32 days after MC38 tumor challenge and subsequent serum transfer as well as anti-CTLA-4 treatment. (m) representative plots and (n) quantification of intratumoral IFN-γ⁺ or Ki-67⁺CD8⁺ T cells. Data are mean±SEM and pooled from two individual experiments (a-j) n=10-11 mice/group. (k-n) n=5-7 mice/group. *, P<0.05; **, P<0.01; ***, P<0.001; ****, P<0.0001.

FIG. 4A-4K: Effect of inosine on T cell differentiation and dependency on classical dendritic cells of ICB therapy efficacy. (a) Scatter plot of untargeted metabolomics data in the serum of anti-CTLA-4 treated, tumor-bearing B.p. monocolonized compared to C.sp. monocolonized and germ-free (GF) mice. Grey circles or dotted grey circles depict inosine or inosine fragments/adducts, respectively. Inset shows an extracted ion chromatogram of inosine (b) Intensity of inosine (AUC: area under the curve) in sera shown in panel (a) of this figure. (c) Naïve CD4⁺ T cells were co-cultured with bone marrow derived dendritic cells and IFN-γ. Quantification of T-bet⁺CD3ε⁺CD4⁺ T cells 48 hours after co-culture in the presence or absence of inosine, A_{2A} receptor inhibitor (ZM241385), cell permeable cAMP (db-cAMP) and protein kinase A inhibitor (RP-8-CPT-CAMPS). (d) Same as (c) without IFN-γ. (e) Representative plot and quantification (left and right panel) of phospho-CREB (Ser133) levels in naïve CD4⁺ T cells cultured with anti-CD3/anti-CD28 coated beads for 1 hour in the presence or absence of inosine. (f) Schematic overview of experimental setup to deplete classical dendritic cells during MC38 tumor challenge and anti-CTLA-4 treatment. Intratumoral (g) frequency of CD8⁺ T cells (h) IFN-γ⁺CD8⁺ T cells. (i and j) same as (g and h), but CD4⁺ T cells and (k) tumor weight at day 95. Data are mean±SEM and pooled from two individual experiments (a-b) n=5-8 samples/group (c-e) 10-15 biological replicates/group (f-k) 10 mice/group. *, P<0.05; **, P<0.01; ***, P<0.001; ****, P<0.0001

FIG. 5A-5L: Inosine promotes Th1 activation and anti-tumor immunity. (a) Schematic overview of experimental setup to assess the effect of inosine on Th1 activation in vivo. (b, c) Representative dot plots and quantification (left and right panel respectively) of T-bet⁺IFN-γ⁺CD3ε⁺ (b) CD8⁺ or (c) CD4⁺ T cells in the MLN. (d) Schematic overview of experimental setup to assess the effect of inosine on anti-tumor immunity. Upon palpable tumors, mice were treated with 100 μg anti-CTLA-4 i.p. (5 times every 72 hours) and in some groups 20 μg CpG i.p. (5 times every 72 hours). In addition, inosine (300 mg/KG/BW) or PBS was given daily orally (O), through gavage or systemically (S) through i.p. injection. (e) Tumor weight and quantification of IFN-γ⁺ cells amongst (f) CD4⁺ or (g) CD8⁺ cells are shown. (h). Schematic overview to assess the requirement of A2_{A}R signaling for inosine-induced anti-tumor immunity. 1×10⁶ MC38 cells (s.c.) and WT or A2_{A}R-deficient 1×10⁷ T cells (i.v. 6×10⁶ CD4⁺ and 4×10⁶ CD8⁺ cells) were injected. Upon palpable tumors, mice were treated with 100 μg anti-CTLA-4, 20 μg CpG (4 times every 72 hours, both i.p.) and inosine (daily, 300 mg/KG/BW, through gavage). (i) Pictures of tumors are shown at day 20. scale bars: 1 cm. (j) Tumor weight and quantification of IFN-γ⁺ in (k) CD8⁺ or (l) CD4⁺ cells in the tumor are shown. Data are mean±SEM and pooled from two individual experiments (a-l) 10-11 mice/group. *, P<0.05; **, P<0.01; ***, P<0.001.

FIG. 6A-6J: ICB therapy efficacy is CRC subtype dependent. (a, c, e and h) Schematic overview of the experimental setups to assess the effect of ICB-promoting bacteria in different subtypes of CRC. (b and d) Survival curve of isotype, anti-CTLA-4 or ICB-promoting (B.p. L.j. O.sp.) or control (C.sp. P.sp.) and anti-CTLA-4 co-treated Apc^{2lox14/+}; Kras^{LSL-G12D/+}; Fabpl-Cre animals. (f) Representative plots and quantification of intratumoral IFN-γ⁺Ki-67⁺ CD8⁺ T cells and (g) tumor weight of Msh2^{LoxP/LoxP} Villin-Cre mice. (i and j) same as (f and g), but for bacteria anti-CTLA-4 co-treated and anti-IL-12p75 co-treated mice as indicated. Data are mean±SEM and pooled from (b and f) five or (d and i) three individual experiments. (b) n=8-9, (d) n=6-7, (f and g) n=10, (i and j) n=7-9 mice/group. *, P<0.05; **, P<0.01.

FIG. 7A-7T: Bacteria are required for ICB therapy efficacy. (a) Schematic overview of the experimental setup to determine if bacteria modulate ICB therapy efficacy. (b) Tumor weight at day 28. Intratumoral (c) representative plots and quantification of (d) IFN-γ⁺ and (e) Ki-67⁺ CD4⁺ T cells at day 28. (f-g) same as (c-e), but for CD8⁺ T cells. Splenic (i) representative plots and quantification of (j) IFN-γ⁺ and (k) Ki-67⁺ CD4⁺ T cells at day 28. (l-n) same as (i-k), but for CD8⁺ T cells. Data are pooled from two individual experiments (n=5-10 mice/group). (o) SPF mice were injected with 1×10⁶ MC38 s.c. and seven days later upon palpable tumors, mice were treated with 100 μg anti-CTLA-4 i.p. (5 times every 72 hours). Mice in the antibiotics (ABX) group received a mix of antibiotics (Ampicillin 1 mg/ml, Colistin 1 mg/ml and Streptomycin 5 mg/ml) orally through the drinking water, starting seven days prior to MC38 cell injection until the end of the experiment, whereas mice in the water group received regular water. Tumors were analyzed three days after the last anti-CTLA-4 injection. (p) Tumor weight, quantification of (q) IFN-γ⁺ and (r) Ki-67⁺ in CD4⁺ T cells at day 25 in the tumor tissue. (s and t) same as (q and r) but CD8⁺ cells. (n=9-10 mice/group). Data are mean±SEM. *, P<0.05; **, P<0.01; ***, P<0.001; ****, P<0.0001.

FIG. 8A-8C: Microbiota dynamics in ICB treated animals and enrichment in fecal samples following ICB treatment. (a) Weighted UniFrac PCoA analysis of 16S rRNA gene V4 region amplicon sequencing in tumors of anti-PD-L1/anti-CTLA-4 (ICB) compared to isotype treated animals. (b) same as (a) but for fecal samples. (c) 16S rRNA gene V4 region amplicon sequencing to identify bacteria in fecal samples of mice treated with ICB or control therapies. Bacteria enriched or reduced in fecal samples of anti-PD-L1/anti-CTLA-4 (ICB) compared to isotype treated animals are shown in green or red respectively. Data are mean±lfcSE (logfoldchangeStandard Error) (n=7-14 mice/group). Statistics: (a) and (b) PERMANOVA, (c) Benjamini-Hochberg

FIG. 9A-9F: B.p. enhances anti-PD-L1 therapy efficacy. (a) Germ-free mice were monocolonized with B.p. or C.sp. Seven days later, 1×10⁶ MC38 cells were injected s.c. and seven days later upon palpable tumors mice were treated with 100 μg anti-PD-L1 i.p. (5 times every 72 hours). Tumors were analyzed three days after the last anti-PD-L1 injection. (b) Tumor weight, quantification of (c) IFN-γ⁺ and (d) Ki-67⁺ in CD4⁺ cells are shown at day 18 in the tumor tissue. (e and f) same as (c and d) but CD8⁺ cells. Data are mean±SEM (n=7 mice/group). *, P<0.05; **, P<0.01; ***, P<0.001.

FIG. 10A-10J: Bacteria alone to not impact on tumor development. (a) Overview of the experimental setup to determine whether B.p. alone have anti-tumor properties. (b) Tumor growth, (c) tumor weight and (d) representative pictures of tumors are shown, scale bars: 1 cm. Intratumoral IFN-γ⁺ (e) CD8⁺ or (f) CD4⁺ T cells at the end of the experiment. Splenic (g) IFN-γ⁺ or (h) Ki-67⁺ CD8⁺ or (i) IFN-γ⁺ or (j) Ki-67⁺ CD4⁺ T cells at the end of the experiment. Data are mean±SEM. (a-j) n=7 mice/group.

FIG. 11A-11B: Bacteria do not translocate into tumor tissue. (a) Representative pictures of SYTOX green nucleic acid stain of feces and tumor tissue of indicated colonized mice 18 days after initiation of anti-CTLA-4 therapy. (b) Agarose gel of full length 16SrRNA amplicons of feces and tumor tissue of indicated colonized mice.

FIG. 12A-12R: Effect of B.p. on T cell differentiation and activation. GF animals were monocolonized with either B.p. C.sp. or left GF for 28 days before analysis. Small intestinal CD3e⁺ CD4⁺ T cells expressing (a) RORγt and IL-17a, (b) RORγt, (c) Foxp3 or (d) naïve T cells (defined as RORγt⁻GATA3⁻Foxp3⁻T-bet⁻). (e) Small intestinal CD3e⁺CD8⁺ T cells expressing T-bet. (f-j) same as (a-e), but MLN. (k-o) same as (a-e), but spleen. (p-q) GF animals were colonized as indicated and after 14 days of colonization treated with anti-CTLA-4 (5 times every 72 hours). Quantification of, (p) T-bet⁺IFN-γ⁺CD3e⁺CD8⁺ or (q) naive (defined as RORγt⁻GATA3⁻Foxp3⁻T-bet⁻) CD4⁺ splenic T cells. (r) Correlation of CD8⁺IFN-γ⁺ and CD4⁺IFN-γ⁺ T cells in tumors of anti-CTLA-4 treated, differently colonized mice. Data are mean±SEM and pooled from two individual experiments (a-q) n=5-11 mice/group. (p) n=46 mice. *, P<0.05; **, P<0.01.

FIG. 13A-13D. Reduced barrier integrity upon anti-CTLA-4 treatment. Serum from monocolonized mice treated with or without anti-CTLA-4 was collected and binding against commensal bacteria was assessed. (a) Systemic IgG2b and IgG1 antibody response upon anti-CTLA-4 treatment in monocolonized mice. (b) Jejunum of B.p. or C.sp. monocolonized mice treated with or without anti-CTLA-4 was collected and barrier integrity was assessed through transepithelial electrical resistance measured in Ussing chambers. (c) Histological inflammation score of small intestinal intestine of B.p. or C.sp. monocolonized mice treated with or without anti-CTLA-4. Blinded scoring by a board-certified pathologist revealed no inflammation (Scale bar=100 μm). (d) Levels of proinflammatory cytokines in the serum of B.p. or C.sp. monocolonized mice with or without anti-CTLA-4 treatment (100 μg i.p. five times every 72 hours) were measured. Serum from DSS-treated SPF mice (2% DSS for 5 days) was used as a positive control for systemic inflammatory cytokines. Data are mean±SEM and pooled from two individual experiments. (a) n=9-13 mice/group. (b) n=6-11 mice/group (c) n=4 mice/group (d) n=5 mice/group (pos. ctrl. n=2 mice). *, P<0.05; **, P<0.01; ***, P<0.001, ****, P<0.0001

FIG. 14A-14F: Systemic anti-tumor immunity upon serum transfer and anti-CTLA-4 treatment. GF animals were challenged with MC38 tumor cells. Ten days later mice received serum (i.v.) of anti-CTLA-4 treated tumor-bearing animals. Mice were then additionally treated with anti-CTLA-4 (3 times every 72 hours). Serum donors were colonized with B.p., C.sp. or remained GF, as indicated. Intratumoral (a) IFN-γ⁺ or (b) Ki-67⁺CD4⁺ T cells. Splenic (c) IFN-γ⁺ or (d) Ki-67⁺CD8⁺ T cells. (e and f) same as (c and d) but CD4⁺ T cells. Data are mean±SEM. (a-f) n=5-8 mice/group. *, P<0.05; **, P<0.01; ***, P<0.001; ****, P<0.0001.

FIG. 15A-15F: Inosine levels in vitro and in vivo. (a) Scatter plot of untargeted metabolomics data in the serum of anti-CTLA-4 treated, tumor-bearing B.p. monocolonized compared to C.sp. monocolonized mice. Grey circle identifies the inosine signal. (b) Scatter plot of untargeted metabolomics data in the serum of anti-CTLA-4 treated, tumor-bearing B.p. monocolonized compared to GF mice. Grey circle identifies the inosine signal. (c) Intensity of inosine (AUC: area under the curve) in culture supernatant of indicated bacteria or BHI medium. (d) Parallel reaction monitoring analysis (HCD set at 50 eV) of inosine comparing observed fragmentation patterns in BHI medium spiked with and without 50 μM inosine as well as B.p. cultured in BHI medium. Extracted ion chromatograms of each respective sample are shown in the right panel. (e) Inosine concentrations in duodenal, jejunal or cecal content of B.p. monocolonized mice and in the serum of B.p. or C.sp monocolonized and anti-CTLA-4 treated mice. (f) Inosine concentrations in the serum of untreated (SPF) tumor-bearing, anti-CTLA-4 i.p. (SPF+ anti-CTLA-4) or anti-CTLA-4 plus antibiotic (SPF+ ABX+anti-CTLA-4) treated SPF colonized mice. Anti-CTLA4 treatment: (100 μg 5 times every 72 hours). Antibiotics: Ampicillin 1 mg/ml, Colistin 1 mg/ml and Streptomycin 5 mg/ml orally through the drinking water for 32 days. Data are mean±SEM and pooled from two individual experiments. (c) n=5 biological replicates/group. (e) n=8-11 mice per group. (f) n=6 samples/group. **, P<0.01; ***, P<0.001, ****, P<0.0001.

FIG. 16A-16J: Context dependent effect of inosine on Th1 T cell differentiation. (a) Naïve CD4⁺ T cells were co-cultured with bone marrow derived dendritic cells without IFN-γ. Quantification of T-bet⁺CD3e⁺CD4⁺ T cells 48 hours after co-culture in the presence or absence of inosine and anti-CTLA-4, as indicated. (b) Naïve CD4⁺ T cells were cultured anti-CD3/anti-CD28 coated beads at a ratio of 1:1 without IFN-γ for 48 hours. Representative plot and quantification of IL12Rβ2 surface expression on CD4⁺ T cells in the presence or absence of inosine (left and right panel). (c) Quantification of T-bet⁺CD3e⁺CD4⁺ T cells 48 hours after co-culture in the presence or absence of inosine, db-cAMP and anti-CTLA-4 as indicated. (d) Naïve, A2_{A}R-deficient CD4⁺ T cells were cultured with anti-CD3/anti-CD28 coated beads at a ratio of 1:1 without IFN-γ. Quantification of T-bet⁺CD3e⁺CD4⁺ T cells 48 hours after co-culture in the presence or absence of inosine or db-cAMP (e) Representative plot and quantification (left and right panel) of pCREB expression of anti-CD3/anti-CD28 bead co-cultured CD4⁺ T cells in the presence or absence of inosine or db-cAMP 1 hour after stimulation. Analysis through flow cytometry. (f and g) Naïve, wild type CD4⁺ T cells were cultured with anti-CD3/anti-CD28 coated beads at a ratio of 1:1. Il12rb2 and Ifng gene transcripts (normalized to Gapdh) were evaluated 24 and 48 hours following inosine (1 mM) stimulation. Expression was normalized to cells treated with medium. Analysis through quantitative PCR assay (h) Naïve CD4⁺ T cells were cultured with anti-CD3/anti-CD28 coated beads at a ratio of 1:1 without IFN-γ for 24 hours. Then inosine or vehicle was added at the indicated concentrations for another 48 hours before T cell differentiation and activation was assessed. (i) Adenosine concentrations in the duodenal-, jejunal- or cecal content of B.p. monocolonized mice and in the serum of B.p. or C.sp. monocolonized anti-CTLA-4 treated mice. (j) Naïve CD4⁺ T cells were cultured with anti-CD3/anti-CD28 coated beads at a ratio of 1:1 without IFN-γ for 24 hours. Adenosine or was added then in the indicated concentrations for another 48 hours followed by T cell differentiation and activation was assessed through flow cytometry. Data are mean±SEM and show pooled data of 2 individual experiments (a and b) n=10-16, (c) n=5-10 biological replicates/group, (d and e) n=6 biological replicates/group, (f and g) n=5 biological replicates/group, (h) n=8 biological replicates/group (i) n=8 mice/group (j) n=6 biological replicates/group. *, P<0.05; **, P<0.01; ****, P<0.0001.

FIG. 17A-17B. Inosine does not directly impact tumor cell viability or condition tumor cells for T cell-mediated killing. (a) MC38 tumor cells were treated with the indicated doses of inosine in vitro for 72 hours. Cell death and survival was assessed through flow cytometry. (b) MC38 tumor cells expressing full length ovalbumin (MC38-OVA) were treated with the indicated doses of inosine in vitro for 72 hours. In parallel, OVA-specific naïve CD4 and CD8 T cells from spleens of OT-II and OT-I mice, respectively, were activated with anti-CD3/anti-CD28 beads and rmIL-2 (20 IU/ml) for 72 hours. Inosine was then washed away from conditioned MC38-OVA cells and fresh medium together with activated T cells were added (100,000 MC38-OVA cells+25,000 CD4 cells+25,000 CD8 cells). 72 hours later, cell death and survival of MC38-OVA cells was assessed through flow cytometry. Grey and black dash-dotted lines indicate MC38-OVA cell viability and death when co-cultured with naïve OVA-specific CD4 and CD8 T cells. Data are mean±SEM and pooled from two individual experiments. n=6 biological replicates/condition. #, & P<0.05, **, P<0.01; (**=0.0001 vs 10 mM, #0.001 vs 10 mM and & 1 vs 10 mM)

FIG. 18A-18H: Classical dendritic cells are required for bacteria dependent effect of ICB. (a) IL-12p70 expression in classical dendritic cells (MHCII⁺CD11c⁺B220⁻CD64⁻) and macrophages (MHCII⁺CD11c⁺B220⁻, CD64⁺) (b) Quantification of IL-12p70 expression in macrophages and cDCs. Classical dendritic cells were depleted with diphtheria toxin (DT) in bone marrow chimeric mice after MC38 tumor challenge, followed by anti-CTLA-4 treatment (see FIG. 4h for experimental setup). Quantification of splenic (c) IFN-γ⁺CD8⁺ or (d) Ki-67⁺ CD8⁺ T cells. (e and f) same as (c and d) but for CD4⁺ T cells. (g and h) 1×10⁶ MC38 cells (s.c.) were injected in GF mice. Seven days later upon palpable tumors, mice were treated with 100 μg anti-CTLA-4 i.p. (5 times every 72 hours) and in some groups 20 μg CpG i.p. (5 times every 72 hours). In addition, inosine (300 mg/KG/BW) or PBS was given daily orally (O), through gavage or systemically (S) through i.p. injection. Quantification of Ki-67⁺ cells is shown. Data are mean±SEM and show pooled data of 2 individual experiments. (a-f) n=10 mice/group. (g-h) n=6-7 mice/group. *, P<0.05 ***, P<0.001; ****, P<0.0001.

FIG. 19A-19F: Bacteria-dependent enhancement of ICB therapy efficacy in Msh2^{LoxP/LoxP} Villin-Cre mice. Msh2^{LoxP/LoxP} Villin-Cre mice were treated with anti-CTLA-4, ICB-promoting or control-bacteria and/or anti-IL-12p75 (see FIGS. 5e and h for a detailed experimental setup) (a) Representative plots and quantification of intratumoral IFN-γ⁺Ki-67⁺CD4⁺ T cells. (b) same as (a), but for bacteria anti-CTLA-4 co-treated and anti-IL-12p75 co-treated animals as indicated. (c) Representative plots and quantification of intratumoral CRC stem cells (defined as Ep-CAM⁺LGR5⁺). (d) same as (c), but for bacteria anti-CTLA-4 co-treated and anti-IL-12p75 co-treated animals as indicated. (e) Representative plots and quantification of tumor infiltrating leukocytes (TILs). (f) same as (e), but for bacteria anti-CTLA-4 co-treated and anti-IL-12p75 co-treated animals as indicated. Data are mean±SEM and pooled from (a, c and e) five or (b, d and f) three individual experiments. (a, c and e) n=10 (b, d and f) n=7-9 mice/group. *, P<0.05; **, P<0.01; ***, P<0.001.

FIG. 20A-20D: Oxaliplatin, anti-PD-L1 co-therapy is enhanced by ICB-promoting bacteria. (a) Schematic overview of the experimental setup to assess the effect of ICB-promoting bacteria in Msh2^{LoxP/LoxP} Villin-Cre mice. 319 days post birth antibiotics were given orally through the drinking water for seven days (Ampicillin 1 mg/ml, Colistin 1 mg/ml and Streptomycin 5 mg/ml). Then Msh2^{LoxP/LoxP} Villin-Cre mice were treated with Oxaliplatin, anti-PD-L1 and ICB promoting (B.p., L.j. and O.sp.) or control bacteria (C.sp. and P.sp.). Bacteria were given 5 times 72 hours apart through gavage, 100 μg anti-PD-L1 was given 5 times 72 hours apart, i.p. Oxaliplatin 2.5 mg/KG/BW was given three times 7 days apart, i.p. (b) Tumor weight of Msh2^{LoxP/LoxP} Villin-Cre mice. (c) Representative pictures of dissected tumors. (scale bar: 1 cm) (d) Quantification of tumor-infiltrating leukocytes (TILs). Data are mean±SEM. n=5-7 mice/group **, P<0.01.

FIG. 21: Mechanism of bacteria-induced ICB efficacy enhancement. ICB-promoting bacteria increase inosine levels systemically, which is linked to an ICB-dependent reduction in gut barrier integrity. Inosine-mediated A_{2A} receptor engagement leads to increased intracellular cAMP, protein kinase A activation and finally phosphorylation of the transcription factor CREB. Together with TCR stimulation, which is further enabled through anti-CTLA-4 treatment, this leads to increased expression of IL12 receptor on T cells. Classical dendritic cells (CDC) sample antigens and are the major cellular source of IL-12. IL-12 produced by cDCs induces Th1 differentiation, through induction of T-bet (Tbx21) expression and activation of T cells. cDCs are required for microbe-anti-CTLA-4 induced IFN-γ (Ifng) production by Th1 T cells, which are protective in cancer.

FIGS. 22-24: The lists of Bifidobacterium sp. (B.sp.), Lactobacillus sp. (L.sp.) and Olsenella sp. (O.sp.). Tables show the sequence ID of Bifidobacterium sp. (B.sp.), Lactobacillus sp. (L.sp.) and Olsenella sp. (O.sp.) with more than 84%-95% identity to the sequences identified in examples (FIGS. 22, 23, and 24, respectively) based on full length 16S sequence.

FIGS. 25-27. 16S rDNA sequence of the administered strains, Bifidobacterium pseudolongum strain deposited as IDAC Deposit No. 231020-01, Lactobacillus johnsonii strain deposited as IDAC Deposit No. 231020-02, and Olsenella sp. strain deposited as IDAC Deposit No. 231020-03, respectively. SEQ ID NO: 1 has 99% identity to the 16S rRNA sequence of Bifidobacterium pseudolongum subsp. globosum strain RU 224. SEQ ID NO: 2 has 99% identity to the 16S rRNA sequence of Lactobacillus johnsonii strain CIP 103620. SEQ ID NO: 3 has 94% Olsenella profusa strain DSM 13989, 94% identity to Olsenella umbonata strain lac31, and 94% identity to Olsenella uli strain DSM 7084.

FIG. 28. Comparison of levels of selected metabolites in transferred serum samples in the serum of mice monocolonized with B.p. compared to C.sp. or GF mice. The purine metabolite inosine was significantly more abundant (8 to 9-fold) in sera from B.p. monocolonized mice compared to sera from C.sp. monocolonized or GF mice. Of note, xanthine and hypoxanthine, degradation products of inosine, were also elevated in the sera of B.p. monocolonized mice.

FIG. 29A-29F. (A) Schematic of the experimental setup, (B) Tumors and tumor weight at the end of the experiment in MC38 tumor bearing and anti-CTLA-4 treated (5 times, 72 hours apart) monocolonized mice. (C) Inosine concentration measured in the serum of mice shown in (B). (D) Hypoxanthine production of indicated bacteria in vitro in BHI media. (E) Tumors and tumor weight at the end of the experiment in MC38 tumor bearing and anti-PD-1 treated (5 times, 72 hours apart) monocolonized mice. (F) Tumors and tumor weight at the end of the experiment in MB49 tumor bearing and anti-CTLA-4 treated (4 times, 72 hours apart) monocolonized mice. Data are mean±SEM. n=4-5 mice/group. (B) One-way ANOVA with Bonferroni post-test. (E and F) students t test. *, P<0.05; **, P<0.01; ***, P<0.001; ****, P<0.0001. The strain labeled B. pseudolongum+Ctrl in FIGS. 30B-30D and as B. pseudolongum in FIGS. 30E-30F are the strain deposited as IDAC Deposit No. 231020-01.

FIG. 30A-30D. (A) Weighted UniFrac PCoA analysis of 16S rRNA gene V4 region amplicon sequencing in feces of anti-PD-L1/anti-CTLA-4 (ICB) compared to isotype treated animals. (B) 16S rRNA gene V4 region amplicon sequencing to identify bacteria in fecal samples of mice treated with ICB or control therapies. Bacteria enriched or reduced in fecal samples of anti-PD-L1/anti-CTLA-4 (ICB) compared to isotype treated animals are shown in green or red, respectively. (C) same as (B) but for tumor samples. Bacteria enriched or reduced in tumors of (D) anti-CTLA-4 and (E) anti-PD-L1 compared to isotype treated animals are shown in green or red, respectively. Panels D and E are the same data as in FIG. 1I but separated by treatment. Data are (A) mean+/−lfcSE (logfoldchangeStandard Error). (A-C) n=5-14 mice/group. *, P<0.05; **, P<0.01; ***, P<0.001; ****, P<0.0001.

FIG. 31A-31E. Bacteria alone do not impact on tumor development. (A) Schematic of the experimental setup, (B) Tumor growth, (C) tumor weight, and quantification of intratumoral IFN-γ+ (D) CD8+ and (E) CD4+ T cells are shown in germ-free (GF) or monocolonized (B. pseudolongum, Colidextribacter species, L. johnsonii, or Olsenella species) MC38 tumor-bearing mice. Data are mean±SEM (B-E) n=5 mice/group. *, P<0.05; **, P<0.01; ****, P<0.0001.

FIG. 32. Inosine levels in vitro. Fold induction compared to media of inosine in culture supernatant of indicated bacteria.

FIG. 33A-33F. B cells and their responses are not required for B. pseudolongum enhanced ICB therapy efficacy. (A) Germ-free (GF) wild type or Igh−/− mice were colonized with B. pseudolongum or left GF. Seven days later, 1×10⁶ MC38 cells were injected s.c. and seven days later upon palpable tumors, mice were treated with 100 μg anti-CTLA-4 i.p. (5 times every 72 hours). Tumors were analysed three days after the last anti-CTLA-4 injection. (B) Tumor weight and quantification of IFN-γ+ in (C) CD4⁺ and (D) CD8⁺ cells are shown at day 18 in the tumour tissue. (E and F) same as (C and D) but Ki-67+ cells. Data are mean+/−SEM. n=4-7 mice/group. *, P<0.05; **, P<0.01; ***, P<0.001; ****, P<0.0001.

FIG. 34A-34H. Akkermansia muciniphila and Lactobacillus johnsonii promote anti-CTLA-4 efficacy and is dependent on T cell expression of A2_{A}R. (A) Schematic overview to assess the requirement of A2_{A}R signaling for Akkermansia muciniphila-induced anti-tumor immunity. Germ-free RAG-1-deficient mice were gavaged with Akkermansia muciniphila and seven days later 1×10⁶ MC38 cells (s.c.) and WT or A2_{A}R-deficient 1×10⁷ T cells (i.v. 6×10⁶ CD4⁺ and 4×10⁶ CD8⁺ cells) were injected. Upon palpable tumors, mice were treated with 100 μg anti-CTLA-4 (4 times every 72 hours). (B) Pictures of tumors (scale bars: 1 cm) and (C) tumor weight are shown at day 27. (D) Quantification of IFN-γ+ in CD8+ or CD4+ cells in the tumor are shown. (E-H) same as (A-D) but in Lactobacillus johnsonii monocolonized mice. Data are mean+/−SEM and (A-H) n=7 mice/group. P<0.05; **, P<0.01; ***, P<0.001.

FIG. 35A-35H. Inosine and live B. pseudolongum improve anti-CTLA-4 therapy efficacy in moderately diverse and complex microbiomes. A) Schematic overview of experimental setup to assess the effect of inosine on anti-tumor immunity in gnotobiotic mice (Oligo-MM12) stably colonized with 12 bacterial species. Upon palpable tumors, Oligo-MM12 colonized mice were treated with 100 μg anti-CTLA-4 i.p. or Isotype antibody (5 times every 72 hours). In addition, inosine (300 mg/KG/BW) or PBS was given daily orally through gavage. (B) Pictures of tumors and (C) tumor weight are shown at day 20. scale bars: 1 cm. (D) Quantification of intratumoral IFN-γ+ cells amongst CD8+ or CD4+ T cells are shown. (E) Schematic overview of experimental setup to assess the effect of inosine and B. pseudolongum on anti-tumor immunity in SPF mice. Following MC38 injection some mice received antibiotics (ABX), specifically Ampicillin 1 mg/ml, Colistin 1 mg/ml and Streptomycin 5 mg/ml for 7 days in the drinking water. Upon palpable tumors, antibiotics were removed and 100 μg anti-CTLA-4 (5 times every 72 hours) started. Mice concomitantly received either PBS, inosine (300 mg/KG/BW daily), B. pseudolongum or heat inactivated (H.i.) B. pseudolongum orally through gavage (5 times every 72 hours). (F) Pictures of tumors and (G) tumor weight are shown at day 20. scale bars: 1 cm. (H) Quantification of intratumoral IFN-γ+ cells amongst CD8+ or CD4+ cells are shown. Data are mean+/−SEM and (A-H) n=7 mice/group. P<0.05; **, P<0.01, ***, P<0.001.

FIG. 36A-36B. Enrichment of Bifidobacteria in tumors of Msh2LoxP/LoxPVillin-Cre mice. SPF Msh2LoxP/LoxPVillin-Cre were treated with 100 μg isotype antibody, anti-CTLA-4 or anti-PD-L1 (5 times every 72 hours) 10 months after birth. Three days following the last treatment, tumor tissues were collected. (A) 16S and (B) Bifidobacteria DNA copy numbers were assessed (normalized to all 16S copy numbers) in the tumor tissue. Analysis through quantitative PCR assay. n=7-11 tumors/group (tumors collected from 4 individual mice in the isotype group and 8 individual mice in the ICB-therapy group). *, P<0.05.

FIG. 37A-37B. Bifidobacteria abundance in responders compared to nonresponding cancer patients. (A) Abundance of B. pseudolongum in fecal samples of non-small cell lung cancer and renal cell carcinoma patients receiving checkpoint blockade therapy (n=37 nonresponders and 44 responders) (8). B. pseudolongum abundance was normalized to nonresponders. (B) Abundance of Bifidobacteria in fecal samples of melanoma patients receiving checkpoint blockade therapy (n=24 nonresponders and 13 responders) (9). Bifidobacteria abundance was normalized to nonresponders.

FIG. 38. Inosine levels in vivo. Inosine concentrations in duodenal, jejunal or cecal content of B. pseudolongum monocolonized mice and in the serum of B. pseudolongum or Colidextribacter sp. monocolonized mice treated with anti-CTLA-4 or anti-PD-L1, as indicated. N=8-11 mice per group. ***, P<0.001, ****, P<0.0001.

FIG. 39. Reduced barrier integrity upon anti-CTLA-4 treatment. Serum from monocolonized mice treated with or without anti-CTLA-4 was collected and binding against commensal bacteria was assessed. N=6-11 mice/group. *, P<0.05; **, P<0.01; ***, P<0.001, ****, P<0.0001.

### DETAILED DESCRIPTION

Generally, the present disclosure provides a compound(s) and/or a compositions for use in treating a subject having cancer, or suspected of having cancer.

In some examples, the cancer may be colorectal cancer (CRC), lung cancer, melanoma, bladder cancer, or kidney cancer. In other examples, the cancer may be breast cancer, prostate cancer, stomach cancer, liver cancer, esophageal cancer, pancreatic cancer, brain cancer, cervical cancer, ovarian cancer, thyroid cancer, lip cancer, oral cancer, larynx cancer, nasopharynx cancer, uterine cancer, or other cancer as disclosed herein.

In a specific aspect, the present disclosure provides a compound(s) and/or a compositions for use in treating a subject having Colorectal cancer (CRC), or suspected of having CRC.

In one aspect, there is described a method of treating a subject having a cancer, or suspect of having a cancer, comprising or consisting of: administering an ICB inhibitor and one or more bacteria selected from Bifidobacterium pseudolongum, Lactobacillus johnsonii, or Olsenella species.

In one aspect, there is described a method of treating a subject having a cancer, or suspect of having a cancer, comprising or consisting of: administering an ICB inhibitor and one or more bacteria selected from Bifidobacterium pseudolongum, Lactobacillus johnsonii, Olsenella profuse, Olsenella umbonata, or Olsenella uli

In a specific example the cancer may be colorectal cancer (CRC), lung cancer, melanoma, bladder cancer, or kidney cancer. In other examples, the cancer may be breast cancer, prostate cancer, stomach cancer, liver cancer, esophageal cancer, pancreatic cancer, brain cancer, cervical cancer, ovarian cancer, thyroid cancer, lip cancer, oral cancer, larynx cancer, nasopharynx cancer, uterine cancer.

In one aspect, there is described a method of treating a subject having CRC, or suspected of having CRC, comprising or consisting of: administering an ICB inhibitor and one or more bacteria selected from Bifidobacterium pseudolongum, Lactobacillus johnsonii, Olsenella profuse, Olsenella umbonata, or Olsenella uli.

In one aspect, there is described a method of treating a subject having CRC, or suspected of having CRC, comprising or consisting of: administering an ICB inhibitor and one or more bacterium selected from Bifidobacterium pseudolongum (B.p.), Lactobacillus johnsonii (L.j), or Olsenella sp. (O.sp.).

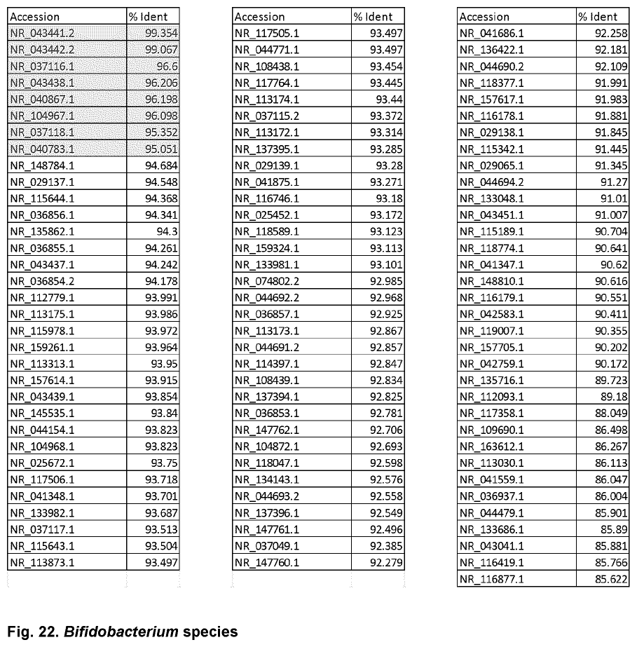

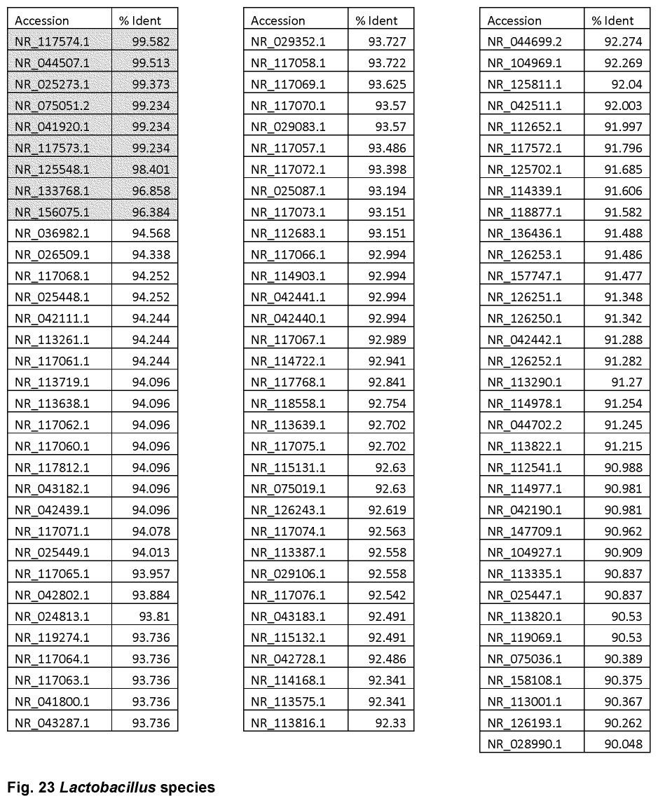

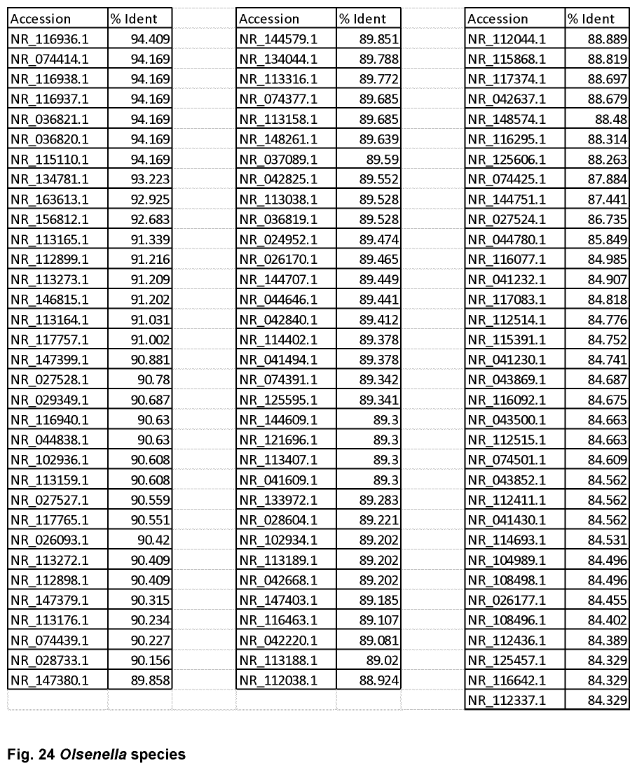

In one aspect, there is described a method of treating a subject having CRC, or suspected of having CRC, comprising or consisting of: administering an ICB inhibitor and one or more bacterium selected from Bifidobacterium sp. (B.sp.) listed in FIG. 22, Lactobacillus sp. (L. sp.) listed in FIG. 23, or Olsenella sp. (O.sp.) listed in FIG. 24.

In one aspect, there is described a method of treating a subject having a cancer, or suspected of having a cancer, comprising or consisting of: administering an ICB inhibitor and inosine, a derivative of inosine, functional derivative of inosine, or a physiologically functional derivative of inosine.

In a specific example the cancer may be colorectal cancer (CRC), lung cancer, melanoma, bladder cancer, or kidney cancer. In other examples, the cancer may be breast cancer, prostate cancer, stomach cancer, liver cancer, esophageal cancer, pancreatic cancer, brain cancer, cervical cancer, ovarian cancer, thyroid cancer, lip cancer, oral cancer, larynx cancer, nasopharynx cancer, uterine cancer.

In one aspect, there is described a method of treating a subject having CRC, or suspected of having CRC, comprising or consisting of: administering an ICB inhibitor and inosine, a derivative of inosine, functional derivative of inosine, or a physiologically functional derivative of inosine.

As used herein, the terms “immune checkpoint,” “checkpoint pathway,” and “immune checkpoint pathway” refer to a pathway by which the binding of an immune checkpoint ligand to an immune checkpoint receptor modulates the amplitude and quality of the activation of immune cells.

Immune checkpoint proteins include, but are not limited to, cytotoxic T lymphocyte-associated antigen 4 (CTLA-4), also known as CD152, programmed cell death protein 1 (PD-1), also known as CD279, PD-1 ligands (PD-L1 or CD274, PD-L2 or CD274), lymphocyte-activation gene 3 (LAG-3), also known as CD223, B7-H3 (CD276), V-domain Ig suppressor of T cell activation (VISTA), therapies targeting indoleamine 2′3′ dioxygenase (IDO, IDO1 and IDO2), TIGIT (also called T cell immunoreceptor with Ig and ITIM domains), B and T Lymphocyte Attenuator (BTLA), Herpes virus entry mediator (HVEM), CD226 (DNAM-1) and CD96 (Tactile), T cell immunoglobulin mucin (TIM-3), also known as HAVcr2, LAIR1 (Leukocyte Associated Immunoglobulin Like Receptor 1; CD305), CD160 (BY55), CD244 (2B4), VTCN1 (B7-H4), KIR, A2_{A}R, or B7-H3.

The term “immune checkpoint blockade” or “ICB,” as used herein, refers to the administration of one or more inhibitors of one or more immune checkpoint proteins or their ligand(s). Thus, the term “immune checkpoint blockade” refers to the inhibition of an immune checkpoint pathway by the administration or expression of a “blockade agent” or “inhibitor”. Typically, the “blockade agent” prevents the interaction of the immune checkpoint receptor and ligand, thereby inhibiting the checkpoint pathway. A blockade agent may be a small molecule, peptide, antibody or fragment thereof, etc. that binds to an immune checkpoint ligand or immune checkpoint receptor and inhibits the formation of the ICR/ICL complex. A blockade agent may also function by preventing signaling by the ICR/ICL complex. Exemplary ICB agents include antibodies, fusion proteins, and small molecules, such as ipilimumab (YERVOY®, anti-CDLA-4 antibody, Bristol-Myers Squibb), nivolumab (OPDIVO®, anti-PD-1 antibody, Bristol-Myers Squibb), pembrolizumab (KEYTRUDA®, anti-PD-1 antibody, Merck), atezolizumab (TECENTRIQ®, anti-PD-L1 antibody, Roche), avelumab (BAVENCIO®, anti-PD-L1 antibody, Merck KGaA/Pfizer), durvalumab (IMFINZI®, anti-PD-L1 antibody, Medimmune/AstraZeneca), cemiplimab (LIBTAYO®, anti-PD-1 antibody, Regeneron/Sanofi), lambrolizumab (anti-PD-1 antibody, Merck), pidilizumab (anti-PD-1 and anti-DLL antibody, Medivation), BMS-936559 (anti-PD-L1, Bristol-Myers Squibb), MEDI-0680 (anti-PD-1 antibody; AMP-514; AstraZeneca), REGN2810 (anti-PD-1 antibody, Regeneron), CA-170 (small molecule PD-1 and PD-L1 inhibitor; Curis), BMS-1166 (small molecule PD-L1 inhibitor, Bristol-Myers Squibb), AMP-224 (anti-PD-1 fusion protein, Medimmune), and spartalizumab (anti-PD-1 antibody, Novartis).

As used herein, the term “immune checkpoint inhibitor” refers to molecules that totally or partially reduce, inhibit, interfere with or modulate one or more checkpoint proteins. Checkpoint proteins regulate T-cell activation or function. These proteins are responsible for co stimulatory or inhibitory interactions of T-cell responses. Immune checkpoint proteins regulate and maintain self-tolerance and the duration and amplitude of physiological immune responses. In some embodiments, the subject can be administered an additional agent that can enhance or boost the immune response, e.g., immune response effected by the binding molecules (e.g., BCMA-binding molecules), recombinant receptors, cells and/or compositions provided herein, against a disease or condition, e.g., a cancer, such as any described herein.

Immune checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors, ligands and/or receptor-ligand interaction. In some embodiments, modulation, enhancement and/or stimulation of particular receptors can overcome immune checkpoint pathway components.

The terms “inhibit,” “block,” and “suppress” are used interchangeably and refer to any statistically significant decrease in biological activity, including full blocking of the activity.

An “inhibitor” is an active agent that inhibits, blocks, or suppresses biological activity in vitro or in vivo.

Inhibitors include but are not limited to small molecule compounds; nucleic acids, such as siRNA and shRNA; polypeptides, such as antibodies or antigen-binding fragments thereof, dominant-negative polypeptides, inhibitory peptides, and fusion proteins; and oligonucleotide or peptide aptamers.

In a specific example, the ICB inhibitor is an anti-CTLA4 antibody, or an anti-PD-L1 antibody, or an anti-PD-1 antibody.

Non-limiting examples of co-stimulants include: Toll like receptor (TLR) signals, for example CpG, LPS, Flagellin; Nucleotide-binding oligomerization domain-like receptors (NLRs), for example, meso-diaminopimelic acid, muramyl dipeptide, ATP, extracellular glucose, crystals of monosodium urate, calcium pyrophosphate dihydrate, alum, cholesterol or environmental irritants; silica; asbestos; UV irradiation and skin irritants; RIG-I-like receptors (retinoic acid-inducible gene-I-like receptors), for example, single- or double-stranded RNA (e.g., from viruses); C-type lectin receptors (CLR), for example, repeated mannose units, C-type lectin domain; Cytokine receptor signalling, for example, IL-12, IL-18, IL-33, IFN-g; Stimulation provided through antigen presenting cells or their counterpart on T-cells, for example, CD80-CD28, CD86-CD28, CD40CD40L, OX-40L-OX40; -cGAS-STING pathway; for example, cytosolic DNA.

A “standard dose” of ICB therapy is known by a person of skill in the art for each medication, and may be the dose that is indicated in the prescribing information and/or the dose that is most frequently administered under particular clinical circumstances (for example for the particular PD-1 inhibitor and/or CTLA-4 inhibitor being used, the particular route of administration being used, the particular stage of the CRC being treated, the age, weight, and/or sex of the particular patient, etc.).

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. Additional examples of domesticated animals include a ferret, horse, rabbit, guinea pig, gerbil, hamster, chinchilla, rat, mouse, or other small mammal; a bird; a reptile; a fish; an amphibian; an arthropod such as a tarantula or hermit crab. Additional livestock animals include donkey, alpaca, camel, water buffalo, mink, or chicken.

In a specific example, the subject is a human.

The terms “colorectal cancer” or “CRC”, used interchangeably herein, are used in the broadest sense and refer to (1) all stages and all forms of cancer arising from epithelial cells of the intestinal tract below the small intestine (i.e., the large intestine (colon), including the cecum, ascending colon, transverse colon, descending colon, and sigmoid colon, and rectum), and/or (2) all stages and all forms of cancer affecting the lining of the large intestine and/or rectum.

In some examples, CRC is mismatch repair deficient (MMRD) CRC or inflammation-associated CRC.

Typically, in the staging systems used for classification of colorectal cancer, the colon and rectum are treated as one organ.

Additionally, as used herein, the term “colorectal cancer” also includes medical conditions which are characterized by cancer of cells of the duodenum and small intestine (jejunum and ileum).

The staging of CRC is known.

In some examples, CRC may be staged according to the Dukes system, the Astler-Coller system or the TNM system (tumors/nodes/metastases), whereby the latter is most commonly used. The TNM system of the American Joint Committee of Cancer (AJCC) describes the size of the primary tumor (T), the degree of lymph node involvement (N) and whether the cancer has already formed distant metastasis (M), i.e., spread to other parts of the body. Here, stages 0, IA, IB, IIA, IIB, III and IV are defined based on the determined T-, N- and M-values. A corresponding staging scheme can be derived from the Cancer Staging Manual of the AJCC. Another system for staging of colorectal cancer is the Dukes system, defining cancer stages A, B, C and D. This system was adapted by Astler and Coller, who further subdivided stages B and C (“modified Astler-Coller classification”).

As used herein, a CRC patient includes patients staged according to any staging system used and irrespective of the stage diagnosed.

As use herein “a patient suffering from colorectal cancer” or “a subject suffering from colorectal cancer” refers to any mammalian, in particular human, patient having developed atypical and/or malignant cells in the lining and/or the epithelium of the large intestine and/or rectum. This includes CRC patients independent of the stage and form of the CRC.

Patients suffering from colorectal cancer also include patients which are recurrent with colorectal cancer, i.e., patients wherein after surgical treatment the tumor could no longer be detected for a certain time span, but wherein the cancer has returned in the same or different part of the large intestine, and/or rectum and/or wherein metastases have developed at different sites of the patient's body such as in the liver, lung, peritoneum, lymph nodes, brain and/or bones.

In another example, the patient suffering from CRC is a patient wherein the initial tumor has already been treated surgically and the CRC is non-metastatic.

In some examples, a derivative of inosine, functional derivative of inosine, a prodrug of inosine, or a physiologically functional derivative of inosine, may be used.

The term “derivative”, “functional derivative” and “physiologically functional derivative” as used herein means an active compound with equivalent or near equivalent physiological functionality to the named active compound when used and/or administered as described herein. As used herein, the term “physiologically functional derivative” includes any pharmaceutically acceptable salts, solvates, esters, prodrugs derivatives, enantiomers, or polymorphs.

The term “prodrug” used herein refers to compounds which are not pharmaceutically active themselves but which are transformed into their pharmaceutical active form in vivo, for example in the subject to which the compound is administered.

The term “therapeutically effective amount” or “effective amount”, as used herein, refers to an amount effective, at dosages and for periods of time necessary to achieve the desired result. Effective amounts may vary according to factors such as the disease state, age, sex and/or weight of the subject. The amount of a given compound or composition that will correspond to such an amount will vary depending upon various factors, such as the given drug or compound, the pharmaceutical formulation, the route of administration, the identity of the subject being treated, and the like, but can nevertheless be routinely determined by one skilled in the art.

The term “treatment” or “treat” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject in the early stage of disease can be treated to prevent progression or alternatively a subject in remission can be treated with a compound or composition described herein to prevent progression.

“Prevent” or “prevention” refers to prophylactic or preventative measures that prevent and/or slow the development of a targeted pathologic condition or disorder. Thus, those in need of prevention include those at risk of or susceptible to developing the disorder. In certain embodiments, a disease or disorder is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the invention.

In some examples, treatment results in prevention or delay of onset or amelioration of symptoms of a disease in a subject or an attainment of a desired biological outcome.

In some examples, treatment methods comprise administering to a subject a therapeutically effective amount of a compound or composition described herein and optionally consists of a single administration or application, or alternatively comprises a series of administrations or applications.

The term “pharmaceutically effective amount” as used herein refers to the amount of a compound, composition, drug or pharmaceutical agent that will elicit the biological or medical response of a tissue, system, animal or human that is being sought by a researcher or clinician, for example, the treatment of colorectal cancer. This amount can be a therapeutically effective amount.

The compounds and compositions may be provided in a pharmaceutically acceptable form.

The term “pharmaceutically acceptable” as used herein includes compounds, materials, compositions, and/or dosage forms (such as unit dosages) which are suitable for use in contact with the tissues of a subject without excessive toxicity, irritation, allergic response, or other problem or complication, commensurate with a reasonable benefit/risk ratio. Each carrier, excipient, etc. is also “acceptable” in the sense of being compatible with the other ingredients of the formulation.

The term “functional derivative” and “physiologically functional derivative” as used herein means an active compound with equivalent or near equivalent physiological functionality to the named active compound when used and/or administered as described herein. As used herein, the term “physiologically functional derivative” includes any pharmaceutically acceptable salts, solvates, esters, prodrugs derivatives, enantiomers, or polymorphs.

In some examples the compounds are prodrugs.

The formulation(s) may conveniently be presented in unit dosage form and may be prepared by any methods well known in the art of pharmacy. Such methods include the step of bringing the active compound into association with a carrier, which may constitute one or more accessory ingredients. In general, the formulations are prepared by uniformly and intimately bringing into association the active compound with liquid carriers or finely divided solid carriers or both, and then if necessary shaping the product.

The compounds and compositions may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intratumoral, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot/for example, subcutaneously or intramuscularly. Preferably compositions comprising bacteria are delivered to the gastrointestinal system, eig., by oral (such as ingestion) or rectal route.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection.

The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

The compounds and/or compositions described herein may be administered either simultaneously (or substantially simultaneously) or sequentially, dependent upon the condition to be treated, and may be administered in combination with other treatment(s). The other treatment(s) may be administered either simultaneously (or substantially simultaneously) or sequentially.

As used herein the term ‘reduces at least one symptom of CRC’ refers to a qualitative or quantitative reduction in detectable symptoms, including but not limited to a detectable impact on the rate of recovery from disease or the rate of disease progression or severity.

As used herein, the term ‘at risk of developing CRC′’ in reference to a subject is understood as referring to a subject predisposed to the development of CRC by virtue of the subject's medical status.

In some example, a subject having CRC′ is a subject having been “diagnosed with CRC′”.

The term ‘diagnosed with CRC′’ refers to a subject demonstrating one or more symptoms of CRC′. Methods of diagnosing CRC′, are known in the art.

The pharmaceutical compositions can be administered in a variety of unit dosage forms depending upon the method of administration. For example, unit dosage forms suitable for oral administration include powder, tablets, pills, capsules and lozenges.

The pharmaceutical compositions described herein may be useful for parenteral administration, such as intravenous administration, intraperitoneal administration, or administration into a body cavity or lumen of an organ or joint.

The term “pharmaceutical composition” refers to a preparation that is in such form as to permit the biological activity of the active ingredient to be effective, and which contains no additional components that are unacceptably toxic to a subject to which the composition would be administered. Pharmaceutical compositions can be administered in any of numerous dosage forms, for example, tablet, capsule, liquid, solution, softgel, suspension, emulsion, syrup, elixir, tincture, film, powder, hydrogel, ointment, paste, cream, lotion, gel, mousse, foam, lacquer, spray, aerosol, inhaler, nebulizer, ophthalmic drops, patch, suppository, and/or enema. Pharmaceutical compositions typically comprise a pharmaceutically acceptable carrier, and can comprise one or more of a buffer (e.g. acetate, phosphate or citrate buffer), a surfactant (e.g. polysorbate), a stabilizing agent (e.g. human albumin), a preservative (e.g. benzyl alcohol), a penetration enhancer, an absorption promoter to enhance bioavailability and/or other conventional solubilizing or dispersing agents. Choice of dosage form and excipients depends upon the active agent to be delivered and the disease or disorder to be treated or prevented, and is routine to one of ordinary skill in the art.

In some examples, the compositions for administration will commonly comprise a solution of the binding agent of the present disclosure dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of binding agents of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin.

Optionally the treatment is combined with another moiety useful for treating CRC.

According to at least some embodiments of the present invention, there is provided use of a combination of the therapeutic agents and/or a pharmaceutical composition comprising same, as recited herein, that can be combined with standard of care or novel treatments for CRC.

For example, treatment methods for a patient suffering from colorectal cancer, in particular after removal of the primary tumor, may include chemotherapy, radiotherapy, targeted therapy, and immunotherapy.

As used herein, the term “chemotherapy” relates to treatment of a subject with an antineoplastic drug.

The terms “radiation therapy” and “radiotherapy” relate to the use of ionizing radiation to treat or control a cancer such as CRC.

The term “targeted therapy”, as used herein, relates to application to a patient a chemical substance known to block growth of cancer cells by interfering with specific molecules known to be necessary for tumorigenesis or cancer or cancer cell growth

The term “immunotherapy” as used herein relates to the treatment of cancer by modulation of the immune response of a subject. Said modulation may be inducing, enhancing, or suppressing said immune response, e.g. by administration of at least one cytokine, and/or of at least one antibody specifically recognizing cancer cells. The term “cell-based immunotherapy” relates to a cancer therapy comprising application of immune cells, e.g. T-cells, preferably tumor-specific NK cells, to a subject.

Whether a patient or a tumor is “responsive,” as used herein with respect to a clinical response to treatment, can be assessed using any endpoint indicating a benefit to the patient, including, without limitation, (1) inhibition, to some extent, of tumor growth, including slowing down and complete growth arrest; (2) reduction in the number of tumor cells; (3) reduction or shrinkage in tumor size; (4) inhibition (i.e., reduction, slowing down or complete stopping) of tumor cell infiltration into adjacent peripheral organs and/or tissues; (5) inhibition of metastasis; (6) enhancement of anti-tumor immune response, possibly resulting in regression or rejection of the tumor; (7) relief, to some extent, of one or more symptoms associated with the tumor; (8) increase in the length of survival following treatment; and/or (9) decreased mortality at a given point of time following treatment. Responsiveness may also be expressed in terms of various measures of clinical outcome. Positive clinical outcome can also be considered in the context of an individual's outcome relative to an outcome of a population of patients having a comparable clinical diagnosis. In one example, an increase in the likelihood of positive clinical response corresponds to a decrease in the likelihood of cancer recurrence.

In another example, clinical response to treatment can be measured based on disease control (DC), wherein tumors displaying disease control include tumors whose response to treatment is a complete response (CR), partial response (PR) or stable disease (SD). In one example, tumors displaying disease control do not include tumors in a progressive disease (PD) state.

In another example, clinical response to treatment can be measured based on an objective tumor response, e.g., tumor shrinkage, wherein tumors undergoing an objective tumor response include tumors undergoing either a complete response (CR) or a partial response (PR). In one embodiment, tumors undergoing an objective tumor response do not include tumors that display stable disease (SD) or tumors in a progressive disease (PD) state.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, “one or more” is understood as each value 1, 2, 3, 4, 5, 6, 7, 8, 9, 10, and any value greater than 10.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### Examples

### Summary

Cancer is a leading cause of death globally. Immune checkpoint blockade therapies offer a promising new therapeutic strategy for many cancers, but have been relatively ineffective for colorectal cancers. Previous studies have shown that the efficacy of immune checkpoint therapies is modulated by the microbiota. Consequently, we hypothesized that specific gut bacteria could be harnessed to promote immunotherapy for colorectal cancer. Herein, we identify three commensal bacterial species and a microbial metabolite, inosine, that enhance the efficacy of immune checkpoint blockade therapy in colorectal cancer. We show that inosine interacts with the adenosine A_{2A} receptor on T cells resulting in intestinal Th1 cell differentiation. Decreased gut barrier function induced by immune checkpoint blockade increased the translocation of bacterial metabolites and promoted cancer protective Th1 cell activation. This novel microbial-metabolite-immune circuit may provide a mechanism for a new class of bacteria-elicited checkpoint blockade therapies. We show that the efficacy of this mechanism differs among the subtypes of colorectal cancer and highlight the strengths and potential limitations of this novel bacterial co-therapy for cancer.

### Methods

### Microbiota Composition by 16S Amplicon Sequencing

DNA extraction and purification from feces and cancer epithelial cells was performed using QIAamp Fast DNA Stool extraction kit (Qiagen). The V4 region of the 16S rRNA gene was amplified with barcoded primers (Kozich et al., 2013) using KAPA HiFi polymerase (Roche) under the following cycling conditions: initial denaturation 98° C. for 2 min, 25 cycles of 98° C. for 30 sec, 55ºC for 30 sec, 72ºC for 20 sec and final elongation at 72ºC for 7 min. NucleoMag® NGS (Macherey-Nagel) was used for PCR clean-up and size selection followed by PCR product normalization with the SequalPrep™ Normalization Plate Kit (ThermoFisher) according to the manufacturer's protocols. Individual PCR libraries were pooled, then qualitatively and quantitatively assessed on a High Sensitivity D1000 ScreenTape station (Agilent) and on a Qubit fluorometer (ThermoFisher). 16S rRNA v4 gene amplicon sequencing was performed using a V2-500 cycle cartridge (Illumina) on the MiSeq platform (Illumina). Sequences were demultiplexed and processed using the dada2 pipeline (Callahan et al., 2016) within R. Forward and reverse reads were trimmed to 230 and 210 base pairs, respectively. Dereplicated sequences were merged, and chimeras were identified and removed using the removeBimeraDenovo function. Taxonomy was assigned using the Greengenes database (DeSantis et al., 2006). Differentially abundant taxa were identified using DEseq2 (Love et al., 2014), fit to the mean (base mean intensity threshold 20) and with Benjamini-Hochberg correction applied to calculate adjusted p-values. For weighted UniFrac analysis, PERMANOVA was used (9999 permutations).

### In Vitro Culture of Bacteria and Full 16SrRNA Gene Sequencing

AOM/DSS tumors were homogenized in sterile culture media (see below) using a steel bead and a TissueLyser II (Qiagen). Homogenized lysate was streaked on brain heart infusion agar (BHI) and Fastidious Anaerobic Agar (FAA), both supplemented with hemin (5 μg/ml), menadione (0.5 μg/ml), mucin (250 μg/ml), cysteine-HCL (250 μg/ml) and Sodium sulfide nonahydrate (250 μg/ml) (all reagents from SIGMA) in anaerobe conditions (Whitley, A95 Workstation). Single colonies were picked 48 hours later and cultivated in BHA or FAA medium containing the same supplements as the agar plates. 48 hours after liquid culture, bacteria were lysed and full-length 16S rRNA PCR was performed followed by standard Sanger sequencing. Bacteria were identified by using BLAST. 16S rRNA full length PCR was performed with the following primers: forward: AGA GTT TGA TCC TGG CTC AG, and a mix of both reverse1: AGA GTT TGA TCA TGG CTC AG, reverse2: ACG GTT ACC TTG TTA CGA CTT.

### Animal Experiments

C57BL/6J and B6(Cg)-Zbtb46^{tm1(HBEGF)Mnz}/J (CDC-DTR)(Meredith et al., 2012) and C; 129S-Adora2a^{tm1Jfc}/J mice were obtained from Jackson and then bred and maintained in house. Apc^{2lox14/+}; Kras^{LSL-G12D/+}; Fabpl-Cre and Msh2^{LoxP/LoxP} Villin-Cre were kindly provided by Dr. Kevin Haigis and Dr. Winfried Edelman. C57BL/6-Tg(TcraTcrb)1100Mjb/J(Hogquist et al., 1994) (OT-I) mice were bred in house. B6.Cg-Tg(TcraTcrb)425Cbn/J(Barnden et al., 1998) (OT-II) mice were kindly supplied by Dr. Markus Geuking. Global B.6-Adora2a^{tm1Jfc}/J (Allard et al., 2019) were obtained from Dr. John Stagg (Barnden et al., 1998). All animals were kept in a 12-hour light-dark cycle on standard 4% fat chow. Offsprings of different SPF (specific pathogen free) breeding pairs were housed together after weaning to minimize cage effects. Germ free C57BL/6J and Rag1^{-/-} mice were bred and maintained in flexible film isolators at the IMC, University of Calgary, Canada. Germ-free status was routinely monitored by culture-dependent and -independent methods and all mice were independently confirmed to be pathogen-free. For experiments, germ-free and monocolonized mice were housed in HEPA filtered Isocages (Tecniplast). Male and female mice between 7-12 weeks were used for experiments. In each experiment mice were age and sex matched and randomly assigned to the different experimental groups. All experiments were performed in accordance with the ethical laws of Alberta and with protocols approved by the Health Sciences Animal Care Committee (AC17-0090 and AC17-0011) following the guidelines set forth by the Canadian Council for Animal Care.

### Single Cell Preparation and Flow Cytometry

Single cells were isolated from spleen, small intestine, mesenteric-, colon draining- and inguinal lymph nodes. Spleen and lymph nodes were cleaned of fat and connective tissue, minced and digested for 20 minutes at 37° C. in a shaking incubator (220 rpm) in RPMI-1640 supplemented with Collagenase type IA (Sigma) 1 mg/ml and DNase I (Roche) 10 IU/ml. Tissues was then filtered through a 40 μm cell strainer (Thermo Fisher) and resuspended in PBS with 2% heat-inactivated fetal bovine serum (FBS) and 2 mM EDTA. Fat, connective tissue and Peyer's patches were removed from small intestines, which was then cut into 0.5-1 cm small pieces. Tissue pieces were washed in pre-warmed calcium- and magnesium-free HBSS (Sigma) containing 5 mM EDTA (Sigma) at 37° C. in a shaking incubator (220 rpm) for 20 min, twice. Supernatant containing intestinal epithelial cells and intraepithelial lymphocytes was discarded. The remaining tissue pieces were then resuspended in in pre-warmed calcium- and magnesium-free HBSS containing Collagenase type VIII (Sigma) 1 mg/ml and digested for 20-25 min at 37° C. in a shaking incubator (220 rpm). Supernatant was filtered first through a 100 μm and then 40 μm cell strainer (Thermo Fisher). For intracellular staining, cells were plated in a 96-well U-bottom plate (Greiner Bio-One) in 200 μl IMDM supplemented with 10% FBS, 0.05 mM 2-Mercaptoethanol, 50 ng/ml Phorbol 12-Myristate 13-Acetate (PMA), 750 ng/ml lonomycin and 10 μg/ml Brefeldin-A (all Sigma) and incubated at 37° C., 5% CO2 for four hours. Cells were then incubated in Fcγ receptor blocking antibody (BD Biosciences) for 10 min at 4° C. followed by surface staining for 25 min at 4° C. For intracellular staining, cells were fixed and permeabilized using the eBioscience™ Foxp3 Fixation/Permeabilization kit (eBioscience) according to the manufacturer's protocol. Cells were then stained with intracellular markers overnight at 4° C. Prior to acquisition, cells were washed and flow cytometry was performed on a FACSCanto (BD Biosciences). Data was analyzed using Flowjo v10.5.3 (Treestar). The antibodies used are tabulated below:

TABLE 1

| | | | | | |
|---|---|---|---|---|---|
| IgG1 | A85-1 | BD | 560089 | APC | RRID: AB_1645625 |
| IgG2b | R12-3 | BD | 743179 | BV786 | RRID: AB_2741330 |
| phospho- | 87G3 | Cell | 9198 | PE | RRID: AB_2561044 |
| CREB | | Signaling | | | |
| (Ser133) | | | | | |
| IL-12 R beta | 305719 | R&D | FAB1959P | PE | RRID: AB_2124048 |
| B220 | RA3-6B2 | BD | 561101 | PerCP/Cy5.5 | RRID: AB_10565970 |
| CD64 | X54- | BD | 558539 | Alexa Fluor | RRID: AB_647120 |
| | 5/7.1 | | | 647 | |

### Colorectal Cancer Models and Treatments

AOM/DSS tumors were induced as previously described in C57BL/6J mice (Mager et al., 2017; Mertz et al., 2016). In brief, AOM (10 mg/kg/BW) (Sigma) was injected twice at day 0 and 19. A 1% DSS (mpbio) in water solution was given to the animals 3 times at day 7, 19 and 29 for 5 days followed by regular water. Isotype, anti-CTLA-4 or anti-PD-L1 antibodies (all Bio X Cell) were injected 5 times every 72 hours (100 μg/injection) intraperitoneally (i.p.), starting at day 122. Apc^{2lox14/+}; Kras^{LSL-G12D/+}; Fabpl-Cre mice have been described previously (Haigis et al., 2008). In short, animals have a median survival of 70 days after birth. Isotype or anti-CTLA-4 antibodies were injected 5 times every 72 hours (100 μg/injection) i.p., starting at day 47. In case of microbial transfer co-therapy, antibiotics (ampicillin 1 mg/ml (Sigma), Colistin 1 mg/ml (Cayman Chemical) and streptomycin 5 mg/ml (Sigma), were mixed with water and given ad libidum for 7 days starting at day 40 post birth. Bacteria were given through oral and rectal gavage 5 times every 72 hours starting at day 47. Tumor development in Msh2^{LoxP/LoxP} Villin-Cre mice has been described before (Kucherlapati et al., 2010). The median survival of Msh2^{LoxP/LoxP} Villin-Cre animals is 365 days after birth. Therefore, we started treatment with isotype, anti-CTLA-4 or anti-IL12p40 (500 μg, Bio X Cell) antibodies 319 days after birth 5 times every 72 hours. In case of microbial transfer co-therapy antibiotics, same as described above, were given for 7 days starting at day 312 and bacteria were supplied orally through gavage 5 times every 72 hours starting on day 319. For heterotopic cancer models, 1×10⁶ cancer cells were injected subcutaneously (s.c.) in the flank of germ-free, monocolonized or SPF mice. Once tumors were palpable (7-10 days post injection) isotype or anti-CTLA-4 antibodies were injected 5 times every 72 hours (100 μg/injection i.p.). For serum transfer experiments, germ-free mice received pooled serum from animals shown in FIG. 2. Serum was transferred 3 times (200 μl serum each time) every 72 hour intravenously (i.v.). Concomitantly to serum transfer, mice received anti-CTLA-4 three times i.p Tumors were measured every 72 hours using a caliper (length×width×height×π/6). All tumors were weighed on a fine scale (Mettler Toledo).

### Dendritic Cell Depletion Experiment

For DC depletion experiments, chimeric mouse generation was adapted from previous reports (Mager et al., 2015; Meredith et al., 2012). In short, C57BL/6J mice were lethally irradiated with 1100 cGy, split into two sessions of 550 cGy each 4 hours apart in a Gamma Cell Exactor 40 (Nordion). Mice were then injected i.v. with 1×10⁷ whole bone marrow from cDC-DTR mice, followed by two weeks of antibiotic treatment in the drinking water (ampicillin 1 mg/ml, Colistin 1 mg/ml and streptomycin 5 mg/ml). Mice then received normal drinking water and were gavaged with a mixture of ICB-promoting bacteria (B.p., L.j., O.sp.). 1×10⁶ cancer cells were injected s.c. in the flank 8 week and DC depletion was initiated with diphtheria toxin (100 ng every 48 hours i.p., Sigma) 9 weeks after irradiation and bone marrow reconstitution). Anti-CTLA-4 was started one day after the first diphtheria toxin injection and given 5 times every 72 hours. Tumors were measured every 72 hours using a caliper (length×width×height×π/6) and weighed at the end of the experiment on a fine scale (Mettler Toledo).

### Cell Culture

MC38 parental strain, MC38-EGFP and MC38-OVA colorectal cancer cells were kindly provided by Dr. Charles Drake. Cells were tested for contamination (Charles River) initially and thereafter screened for absence of Mycoplasma every 6-8 weeks (PCR Mycoplasma detection kit, Thermo Scientific). MC38 cell were maintained at 37° C. under 5% CO2 in IMDM supplemented with 10% heat-inactivated FBS (Sigma), 100 units/ml penicillin, 100 μg/ml streptomycin sulfate, 2 mM L-glutamine, 1 mM sodium pyruvate and non-essential amino acids (all Thermo Fisher). MB49 and B16F10 cells were cultured in IMDM supplemented with 10% FBS (Sigma), 100 units/ml penicillin.

Bone marrow derived dendritic cells (BMDCs) were generated from flushed bone marrow cells, maintained in RPMI-1640 (Sigma) supplemented with 10% FBS, 50 μm 2-Mercaptoethanol (Sigma) 100 units/ml penicillin, 100 μg/ml streptomycin sulfate and 20 ng/ml rm GM-CSF (R&D). Medium was exchanged after 48 hours and 72 hours. 5 days after culture, a magnetic cell sorting step was performed to enrich for CD11c⁺ cells (Miltenyi Biotec). CD11c⁺ cells were seeded in 96 flat bottom wells and pulsed with 20 ng/ml OVA₃₂₃₋₃₃₉ and 100 ng/ml LPS (both Sigma) for 18 hours. In some conditions BMDCs were also cultured with 10 ng/ml rmIFN-γ (R&D).

Negative selection magnetic cell sorting (Miltenyi Biotec) was used to enrich naïve OT-II CD4⁺ T cells. Naïve OT-II CD4⁺ T cells were then co-cultured with BMDCs at a ratio of 2:1 or stimulated with anti-CD3/anti-CD28 T cell activation beads (Thermofisher) at a ratio of 1:1 for 48 hours prior to restimulation with PMA/lonomycin in the presence of Brefeldin-A and analysis (see Single cell preparation and flow cytometry for details). In some conditions cells were additionally cultured with various combinations of 2 μg/ml anti-CTLA-4, 100 μM db-cAMP (Sigma), 5 μM ZM 241385 (Sigma), 300 μM Rp-8-CPT-CAMPS (Cayman Chemical) or 2 mM inosine (Sigma) as described previously (He et al., 2017; Yao et al., 2013).

### Quantitative PCR

Naïve CD4 T-cells were MACS-purified (Miltenyi) according to the manufacturer's protocol. RNA was purified using TRI-reagent (Sigma-Aldrich). RNA was transcribed into cDNA using iScript™ (BioRad). PerfeCTa SYBR Green (Quanta Bio) was used to detect the target genes Il2rb1, Ifng, and Gapdh (QIAGEN). Expression levels of genes were normalized to Gapdh mRNA, and medium versus inosine stimulated groups were compared applying the 2^{-ΔΔCT} method.

### Evaluation of Intestinal Barrier Function

Ussing chamber measurements were performed as previously described (Mager et al., 2017). In brief, one intestinal section (approximately 3 cm long) per mouse was collected from the middle of the small intestine, taking care to exclude Peyer's patches. Electrical resistance was measured in 37% oxygenized HBSS after approximately 10 to 15 min of equilibration time.

Anti-commensal serum antibodies were measured as described before (Mager et al., 2017). Briefly, B.p. or C.sp. were cultured in anaerobe conditions and then diluted to an O.D.600 of 0.07. Bacteria were then inactivated using sodium-azide. Serum from germ-free, B.p. or C.sp. monocolonized mice, treated with or without anti-CTLA-4 was heat-inactivated and incubated with bacterial pellets. Fluorescent secondary antibodies against IgG1 and IgG2b were then used to detect systemic antibodies against pure cultured bacteria. Serum cytokines were by Multiplexing LASER Bead Technology (Eve Technologies).

### Metabolomic Profile Assessment

Metabolites in serum or bacterial cultures were extracted in 50% methanol, centrifuged, and the resulting supernatants were diluted into a linear range for mass spectrometry analysis (1:20 final dilution for microbial cultures and 1:50 total dilution for serum). Ultra-high performance liquid chromatography mass spectrometry (UHPLC-MS) data were then acquired on a Q Exactive™ HF Mass Spectrometer (Thermo Scientific) in negative ion full scan mode (50-750 m/z) at 240,000 resolution. Metabolites were separated via UHPLC using a binary solvent mixture of 20 mM ammonium formate at pH3.0 in LC-MS grade water (Solvent A) and 0.1% formic acid (% v/v) in LC-MS grade acetonitrile (Solvent B) in conjunction with a Syncronis™ column (Thermo Fisher Scientific 97502-102130). Samples were analyzed using a flow rate of 600 uL/min using the following gradient: 0-2 mins, 100% B; 2-7 mins, 100-80% B; 7-10 mins, 80-5% B; 10-12 mins, 5% B; 12-13 mins, 5-100% B; 13-15 mins, 100% B. For all runs the sample injection volume was 2 uL. Metabolite data were analyzed using the XCMS (Gowda et al., 2014; Tautenhahn et al., 2012) and MAVEN software packages (Clasquin et al., 2012; Melamud et al., 2010). Metabolites were identified by matching observed m/z signals (+/−10 ppm) and chromatographic retention times to those observed from commercial metabolite standards (Sigma). Inosine assignments, a key metabolite for this study, were confirmed via MS/MS fragmentation patterns using parallel reaction monitoring. These assignments were further validated by spiking inosine standards into microbial extracts to demonstrate co-retention and matching fragmentation patterns between the observed biomarker and a 50 UM inosine standard.

### Effect of Inosine In Vivo

To evaluate the effect of inosine on Th1 activation, mice received 30 μg CpG, 100 mg EndoFit Ovalbumin (both Invivogen) and 2 μg OVA₃₂₃₋₃₃₉ (Sigma) i.p. and 24 hours later mice received 300 mg/kg/BW inosine or PBS as a control through i.p. injection. T cell differentiation was assessed 48 hours later. To assess the impact of inosine on tumor development during ICB therapy 1×10⁶ cancer cells were injected subcutaneously (s.c.) in the flank of germ-free mice. Once tumors were palpable (7-10 days post injection) 100 μg anti-CTLA-4 antibodies and 20 μg CpG were injected 5 times every 72 hours injection i.p. 24 hours following the first anti-CTLA-4/CpG treatment mice received 300 mg/kg/BW inosine daily orally (gavage) or systemically (200 μl i.p. and 50 μl s.c.) until the end of the experiment. WT or A2_{A} deficient cells were isolated from spleens using CD4/CD8 (TIL) MicroBeads (Miltenyi). T-cell purity was >95% and 1×10⁷ T-cells were transferred i.v.

### Bacterial Detection

SYTOX green nucleic acid stain (Thermo Fisher) was performed according to the manufacturer's instructions. In brief, homogenized feces or tumor tissue was fixed in a 4% paraformaldehyde solution (Sigma) for 30 minutes. Subsequently, samples were diluted in PBS at a ratio 1:5 and stained with SYTOX green nucleic acid stain for 60 minutes prior to picture acquisition (Leica DM2500). 16SrRNA full length PCR was performed as described in above (“In vitro culture of bacteria and full 16SrRNA gene sequencing”).

### Statistical Analysis

GraphPad Prism v.5.04 for Windows was used. If variance between groups was similar parametric tests were used, such as standard student t test or one-way ANOVA with Bonferroni post-test, in case of significantly different variance between groups non-parametric tests, such as Mann Whitney U test or Kruskal Wallis with Dunn's post-test, were applied. Two-way ANOVA with Bonferroni post-test was used for tumor growth curves. Survival was analyzed using the Mantel-Cox Log-rank test. Other tests are denoted in the corresponding figure or table legend. Only statistically significant differences are indicated in the figures. For all statistical analyses: *, P<0.05; **, P<0.01; ***, P<0.001; ****, P<0.0001. Exact P values and statistical tests used for each panel are reported in the source data.

### Results

### ICB Therapy Efficacy Depends on the Gut Microbiota

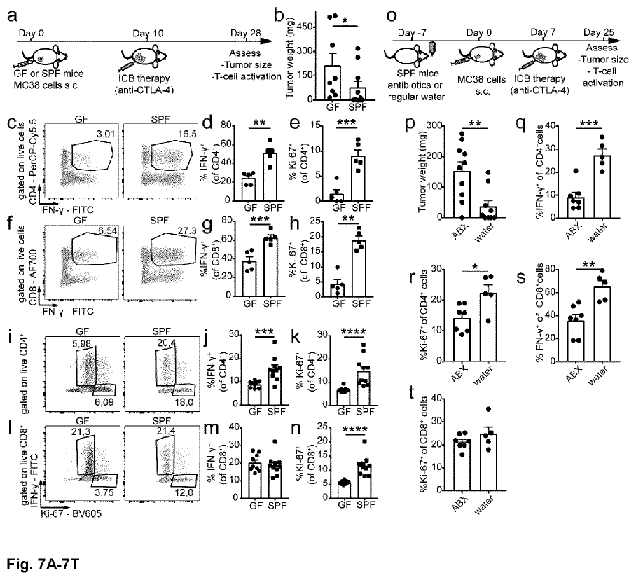

We first questioned whether ICB therapy efficacy in CRC is dependent on the microbiota. Heterotopic MC38 colorectal cancers were implanted into germ-free (GF) and specific pathogen free (SPF) mice and, upon palpable tumor development, ICB therapy was initiated, which led to smaller tumors in SPF animals (FIGS. 7a and 7b). Moreover, intratumoral and splenic CD4⁺ and CD8⁺ T cell activation and proliferation were markedly increased in SPF animals (FIG. 7c-7n). To ensure this was not merely a reflection of the immature immune system of germ-free mice, we also assessed the effect and ICB therapy in antibiotic-treated SPF mice (FIG. 7o). Compared to control treatment, broad spectrum antibiotics also reduced ICB therapy efficacy in tumor-bearing SPF mice (FIG. 7p-7t). These results indicated that ICB efficacy is enhanced in the presence of microbes, corroborating previous reports with other tumor types (Vetizou et al., 2015).

### Identification of ICB-Promoting Bacteria in CRC

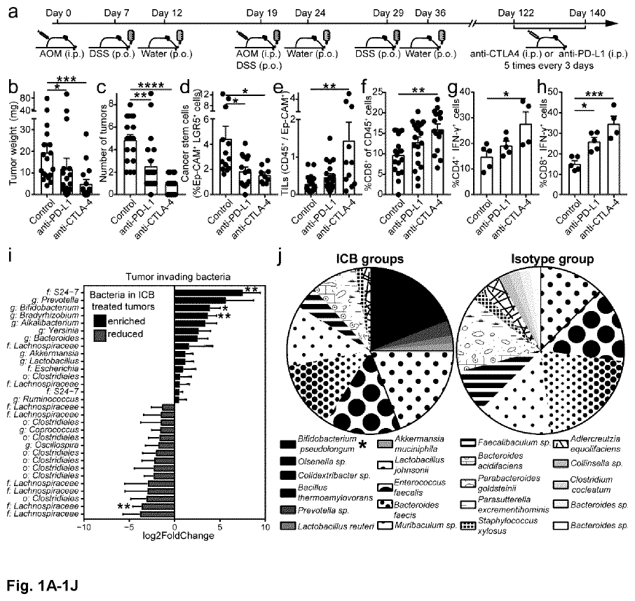

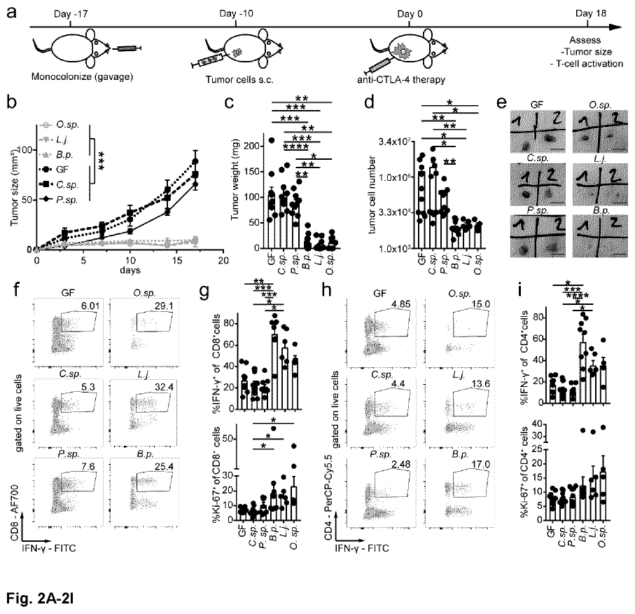

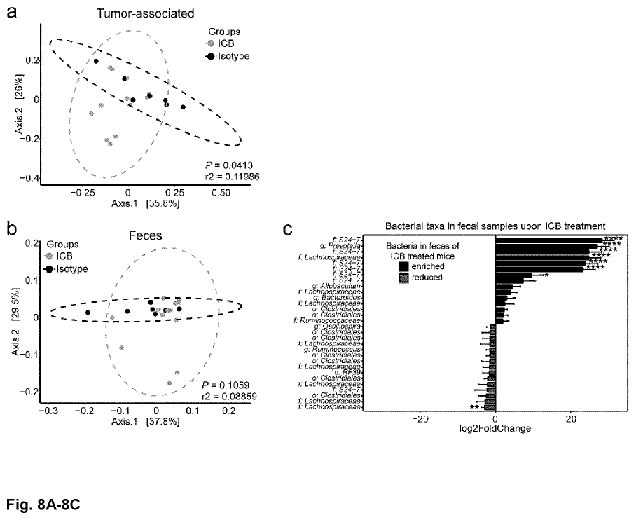

Clinically, ICB therapies are notoriously ineffective in most CRC cases (Le et al., 2015) and heterotopic tumors may not adequately model the spatially close interactions between the gut microbiota and local immunity in intestinal tumors. We therefore employed a more physiological model of CRC to investigate the interactions between the microbiota and immunity in the context of ICB therapy. Intestinal tumors were induced using azoxymethane (AOM) and dextran sulfate sodium (DSS) in SPF animals. Following tumor development, we evaluated the ability of ICB therapy to induce anti-tumor immunity (FIG. 1a). Notably, ICB therapy led to smaller and fewer tumors (FIGS. 1b and c), reduced cancer stem cell numbers (FIG. 1d), increased immune cell infiltration into the tumors (FIG. 1e), and increased CD8⁺ T cell frequencies in the tumor draining lymph node together with increased splenic CD4⁺ and CD8⁺ T cell activation (FIG. 1f-h). In this model, anti-CTLA-4 tumoricidal effects were greater than those induced by anti-PD-L1 treatment when using the same antibody dose. In order to identify potentially beneficial tumor-associated bacteria, we performed 16S rRNA gene V4 region amplicon sequencing of genomic DNA isolated from homogenized tumors as well as anaerobic culture of homogenized tumor tissue. Microbial sequencing revealed that the tumor-associated bacterial community composition of ICB-treated tumors differed from that of control-treated tumors (FIG. 8a and FIG. 1i). Furthermore, we were able to culture twenty-one different bacterial species from tumor tissues. Notably, seven of these cultured bacteria were found only in the ICB-treated group, whereas four were found only in the control group (FIG. 1j).

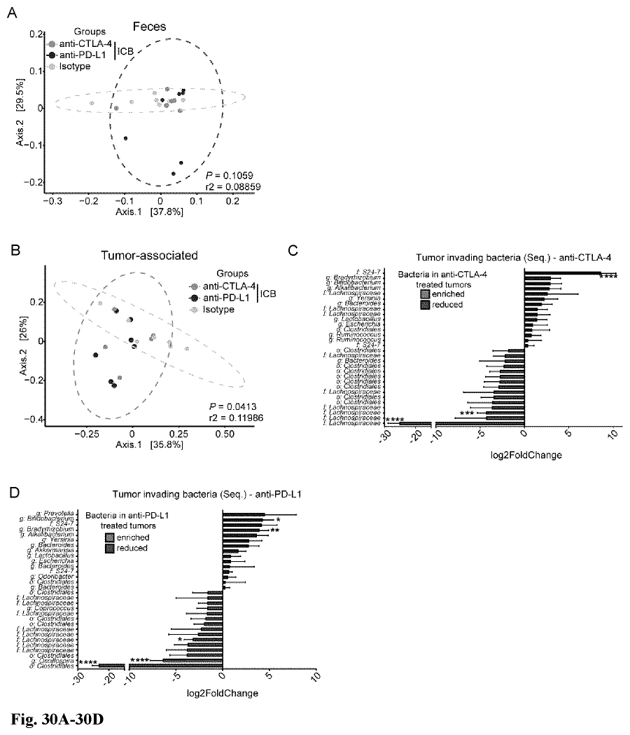

Although no significant changes were observed in the overall fecal bacterial composition (β-diversity) between ICB-treated and control mice (FIG. 30A), a few bacterial families were differentially abundant (FIG. 8C). In contrast, sequencing of tumor-associated bacterial communities revealed differences in B-diversity (FIG. 30B) and additional bacterial genera were differentially abundant in the ICB-treated tumors (FIGS. 30C and 30D).

Interestingly, Akkermansia muciniphila, which was recently identified to enhance the efficacy of anti-PD-L1 and anti-PD-1 treatments in lung and kidney cancers (Routy et al., 2018), was one of the seven bacteria cultured only from ICB-treated tumors. We also performed 16S rRNA gene V4 amplicon sequencing of fecal samples from control and ICB groups but found no significant differences in microbiota composition (FIG. 8b-8c), indicating that the tumor-associated bacterial communities provided a better source for identification of ICB-promoting bacteria in CRC.

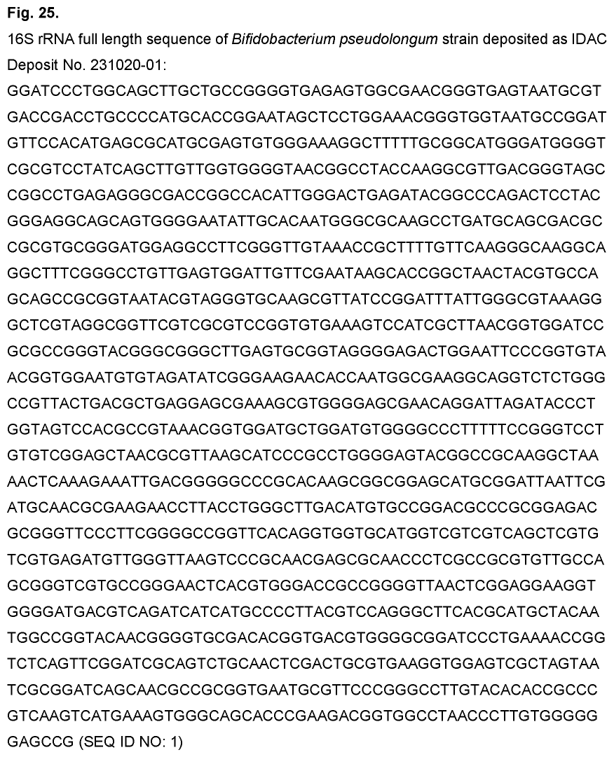

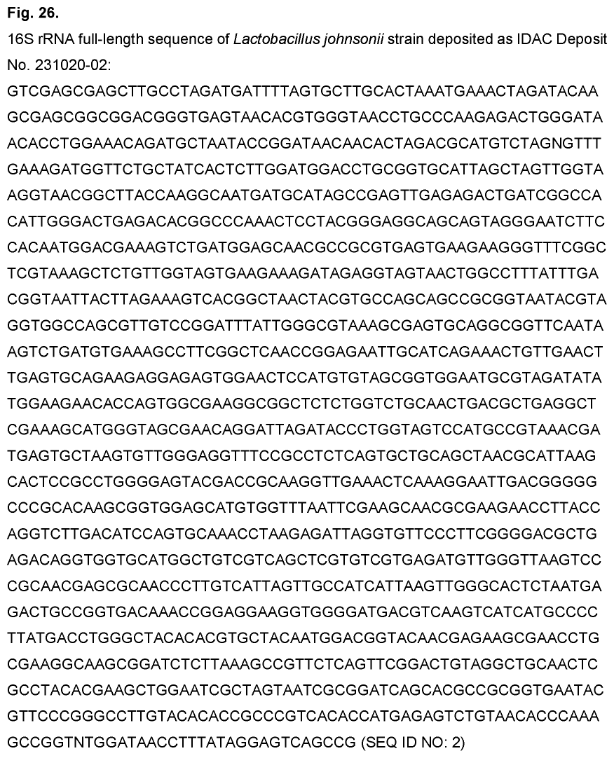

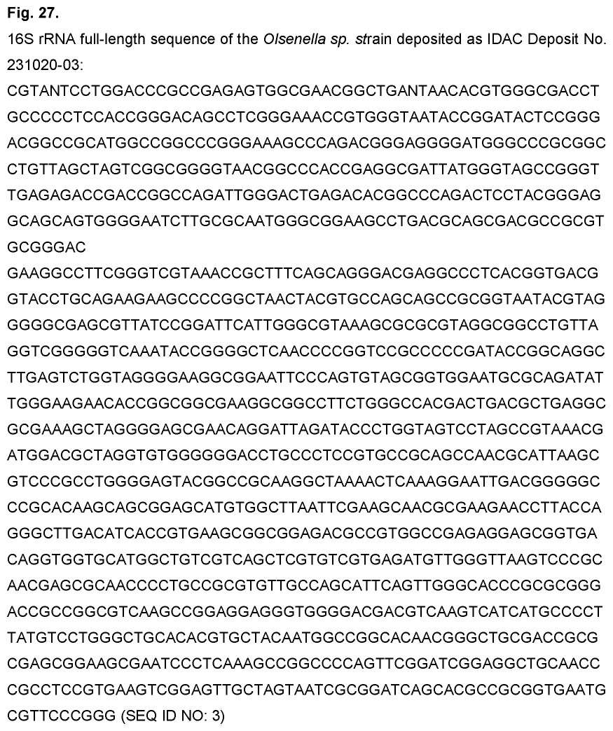

To address whether the bacteria that were found to be enriched in the ICB-treated tumors were able to boost the efficacy of ICB therapy, we selected five of the isolated culturable bacterial species for monocolonization of GF mice. Monocolonized or GF mice were injected with MC38 tumor cells, treated with anti-CTLA-4 upon palpable tumor development and assessed for effects on tumor growth and anti-tumor immunity (FIG. 2a). We chose the heterotopic model of CRC for this approach as the development of orthotopic CRC is severely reduced in animals with a limited microbiota (Schwabe and Jobin, 2013). Of the five bacteria tested, monocolonization with Bifidobacterium pseudolongum (B.p.), Lactobacillus johnsonii (L.j.), and Olsenella sp. (O.sp.) significantly enhanced the efficacy of anti-CTLA-4 treatment compared to GF mice or mice monocolonized with Colidextribacter sp. (C.sp.) or Prevotella sp. (P.sp.) (FIG. 2b-e). The rDNA sequence of the administered Bifidobacterium pseudolongum strain, Lactobacillus johnsonii strain, and Olsenella sp. strain are shown in FIGS. 25-27. Said strains were deposited with the International Depositary Authority of Canada (IDAC, located at National Microbiology Laboratory, Public Health Agency of Canada, 1015 Arlington Street, Winnipeg, Manitoba, Canada R3E 3R2) as follows: Bifidobacterium pseudolongum, IDAC deposit no. 231020-01, deposited on Oct. 23, 2020; Lactobacillus johnsonii, IDAC deposit no. 231020-02, deposited on Oct. 23, 2020; and Olsenella sp., IDAC deposit no. 231020-03, deposited on Oct. 23, 2020.

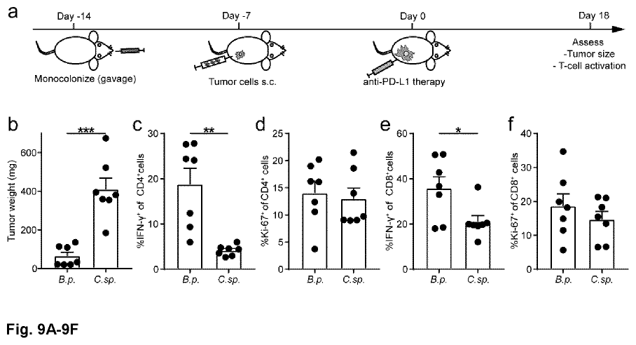

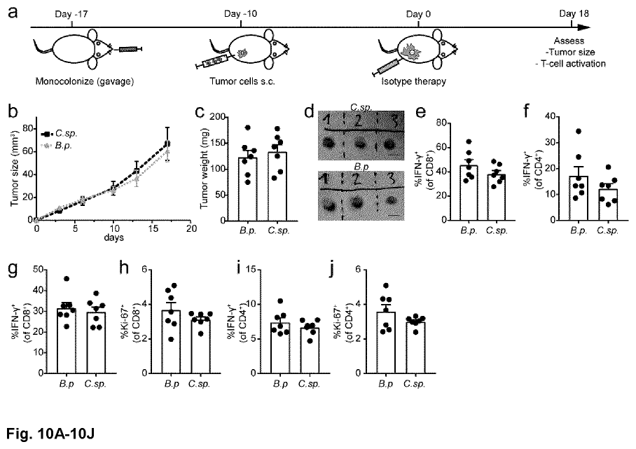

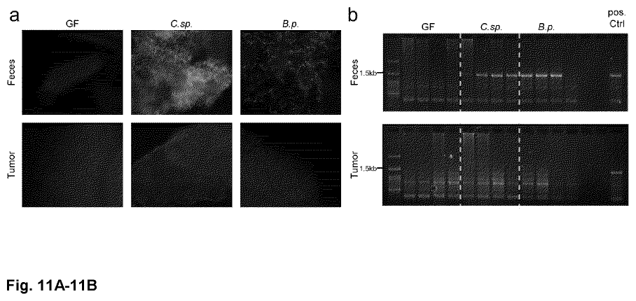

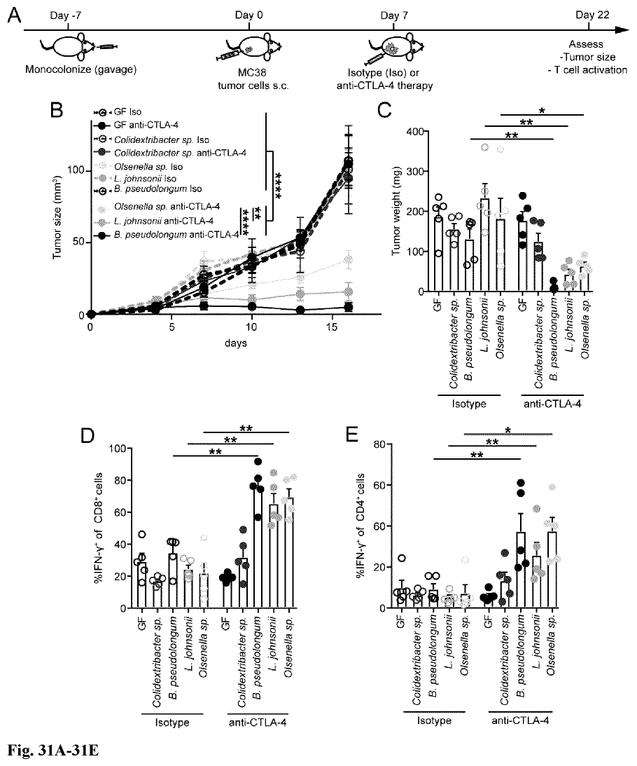

In addition, CD4⁺ and CD8⁺ T cell activation and proliferation were substantially increased in the tumors of B.p., L.j., and O.sp. monocolonized animals (FIG. 2f-i). The isolated ICB-promoting B.p. strain also improved the efficacy of anti-PD-L1 treatment in the MC38 heterotopic tumor model compared to the C.sp. control strain (FIG. 9), albeit to a lower extent than observed for anti-CTLA-4 treatment (at the same dose), which is similar to our observations in the AOM/DSS model. Due to its greater observed efficacy, we performed all subsequent mechanistic studies using anti-CTLA-4 treatment. Of note, anti-tumor immunity was dependent on anti-CTLA-4 or anti-PD-L1 co-therapy as monocolonization with B.p. alone was not able to reduce tumor growth (FIG. 10a-10d and FIG. 31A-31C) or induce anti-tumor immunity (FIG. 10e-10j and FIG. 31D-31E), similar to previous studies with other ICB-promoting bacteria (Routy et al., 2018; Vetizou et al., 2015).

### Induction of Th1 Immunity Through ICB Promoting Bacteria

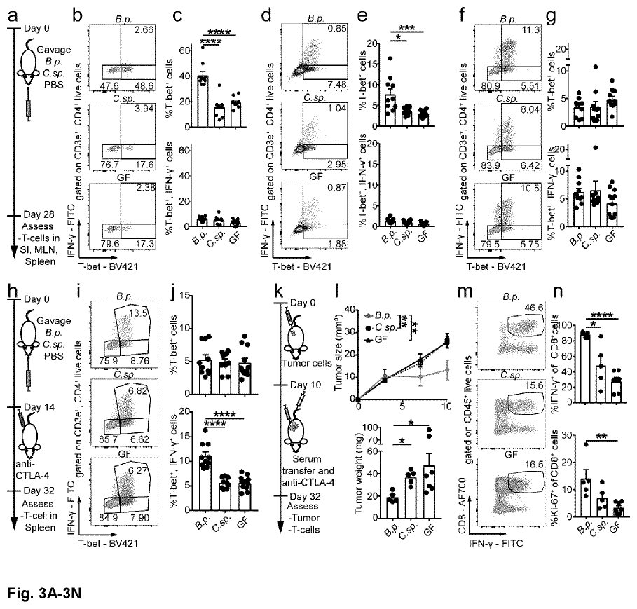

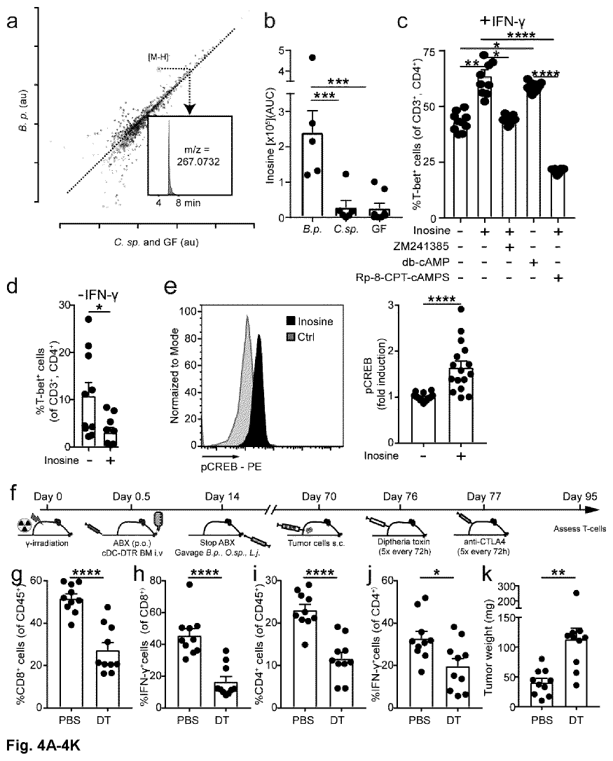

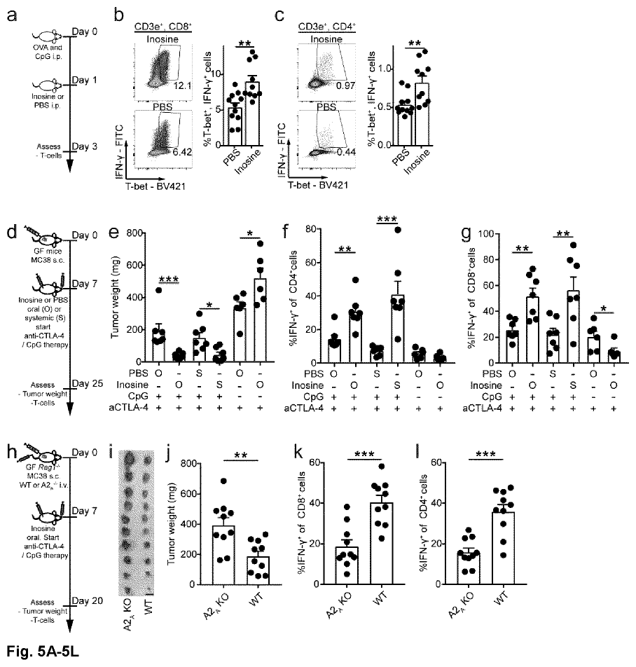

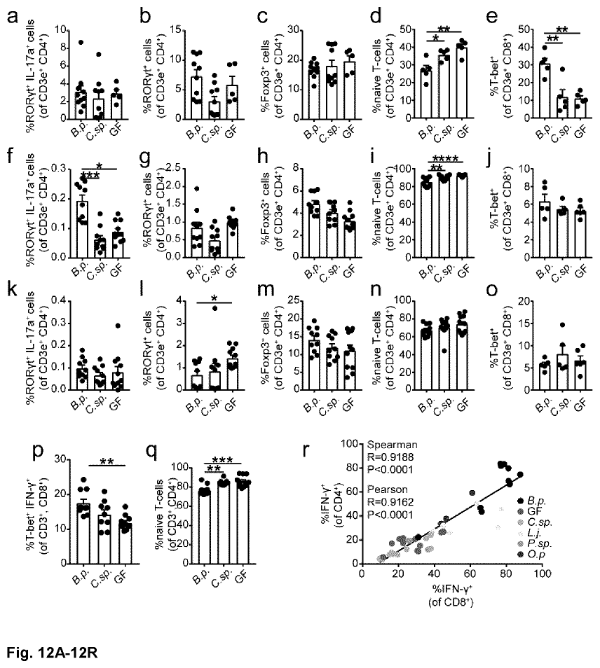

To investigate the mechanism by which the identified bacteria enhanced ICB therapy we selected B.p. as a representative of the beneficial bacteria since it appeared to have the strongest ICB-promoting effect. GF or C.sp. monocolonized served as negative controls. Previous studies revealed the ability of some bacteria to accumulate in the tumor environment where they locally stimulate the immune system and kill tumor cells through toxic metabolites (Zheng et al., 2018). Although bacteria were abundantly present in the feces of B.p. and C.sp. monocolonized mice, we were unable to detect bacteria or amplify 16S rDNA from heterotopic tumors of these mice (FIG. 11), indicating that the beneficial effect of bacteria in this model does not require bacteria to reside within the tumor itself. Compared to GF or C.sp. monocolonized mice, B.p. monocolonization induced a significant increase in expression of the Th1 master transcription regulator T-bet in small intestinal Lamina propria CD4⁺ T cells. Similarly, albeit to a lower extent, B.p. induced T-bet expression in CD4⁺ T cells in the mesenteric lymph nodes (MLN), but not in the spleen (FIG. 3a-g). Intriguingly, B.p. did not activate the effector function of Th1 cells as T-bet⁺⁻IFN-γ⁺ double positive cells did not differ between B.p, C.sp. or GF groups in any of the tissues assessed. Taken together, in the absence of tumors and ICB therapy, B.p. alone promoted Th1 transcriptional differentiation without increasing effector function locally in the gut and draining lymph nodes but not systemically. While B.p. had no effect on other CD4⁺ T cell subsets in the small intestine, it also increased CD8⁺T-bet⁺T cells (FIG. 12a-12e. Moreover, B.p. had minimal impact on Th17 and Treg cells in the MLN and spleen (FIG. 12f-12o).

### Systemic Effect of ICB Promoting Bacteria

Since B.p. alone promoted only local and not systemic Th1 differentiation during homeostasis, we next asked whether the combination of B.p. monocolonization and anti-CTLA-4 therapy (in the absence of a tumor) would induce systemic Th1 activation. Indeed, when combined with anti-CTLA-4, B.p. was able to significantly enhance splenic Th1 cell activation and effector function as evidenced by IFN-γ production compared to C.sp. monocolonized or GF animals (FIG. 3h-j, FIGS. 12p and 12q). We concluded that B.p. induces Th1 differentiation and, together with anti-CTLA-4, activation of Th1 T cells. Interestingly, a recently defined consortium of eleven bacteria was found to induce IFN-γ production preferentially in CD8⁺ T cells and promote anti-tumor immunity in the absence of immunotherapy (Tanoue et al., 2019). In contrast, B.p.-induced IFN-γ production in both CD4⁺ and CD8⁺ T cells (FIG. 12r), and ICB treatment was required for tumoricidal function.

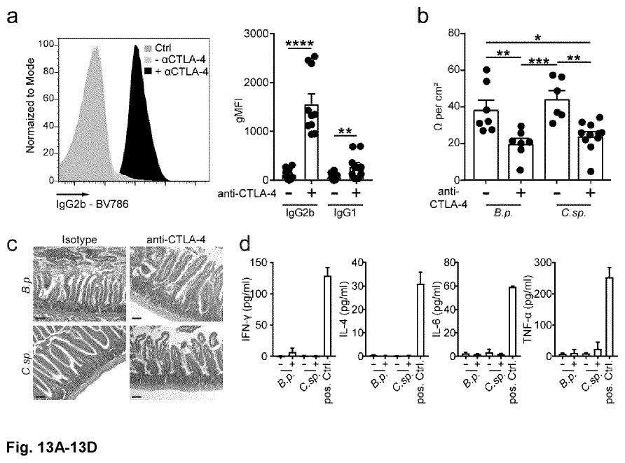

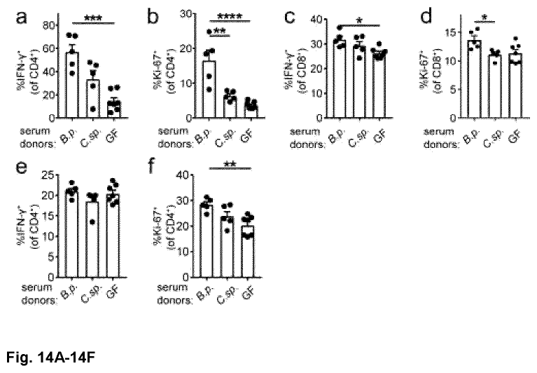

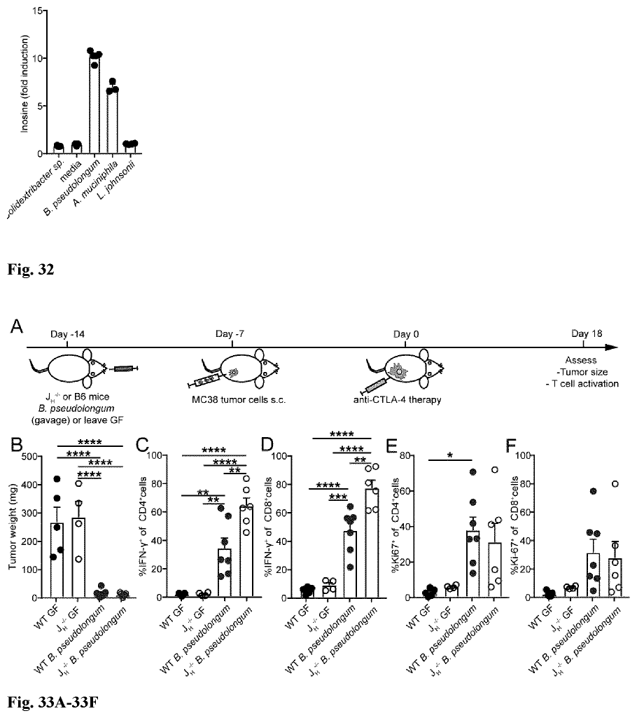

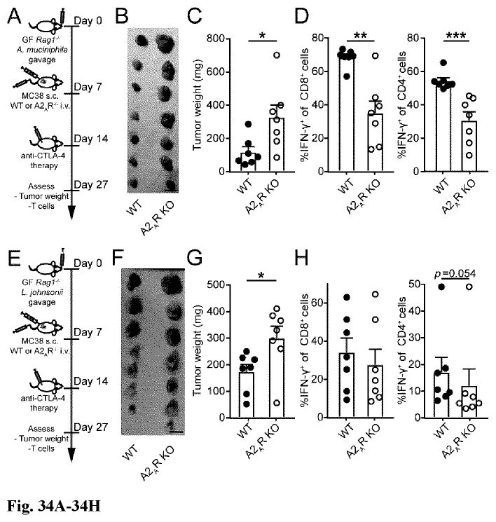

We were intrigued by the ability of B.p. to induce Th1 transcriptional differentiation during homeostasis versus activation of effector function following ICB treatment. Gastrointestinal inflammation is a common immune-related adverse effect of anti-CTLA-4 treatment (Hodi et al., 2010) and we reasoned that this may be due to alterations in gut barrier integrity. Indeed, animals treated with anti-CTLA-4 had increased systemic serum anti-commensal antibodies, particularly Th1-associated IgG2b (Germann et al., 1995), and reduced small intestinal transepithelial electrical resistance compared to controls (FIGS. 13a and 13b; FIG. 39). Although anti-CTLA-4 treatment caused impairment of intestinal barrier integrity it did not induce local or systemic inflammation (FIGS. 13c and 13d). The induction of systemic anti-bacterial antibodies following ICB therapy was not required for the ICB-promoting effect as anti-CTLA-4 treatment was also effective in B. pseudolongum monocolonized mice deficient in B cells and antibodies (FIG. 33). Since bacteria did not accumulate in the (heterotopic) tumors and anti-CTLA-4 reduced the integrity of the gut barrier, we hypothesized that increased systemic translocation of metabolites may be responsible for the systemic effect of B.p. during ICB therapy. To address this, we collected serum from tumor-bearing GF, B.p. or C.sp. monocolonized mice treated with anti-CTLA-4 (see FIG. 2a) and transferred it concomitantly with anti-CTLA-4 into GF MC38 tumor-bearing mice. Remarkably, serum from CTLA-4-treated B.p. monocolonized mice, but not from GF or C.sp. monocolonized mice, was sufficient to reduce tumor growth and elicit strong anti-tumor immunity in the tumor and spleen of GF mice (FIG. 3k-n, FIG. 14a-14f). In sum, these data show that soluble factors derived from or induced by B.p. were responsible for the observed ICB-promoting effects.

### Molecular Mechanism Underlying Th1 Immune Cell Differentiation

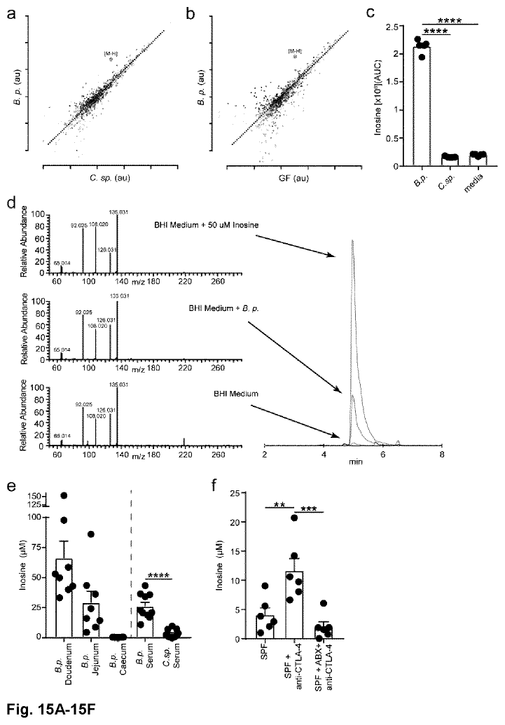

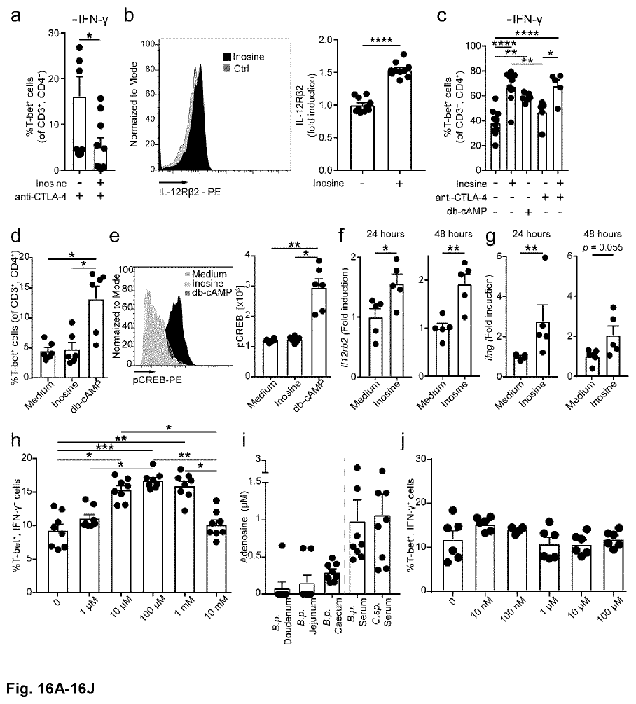

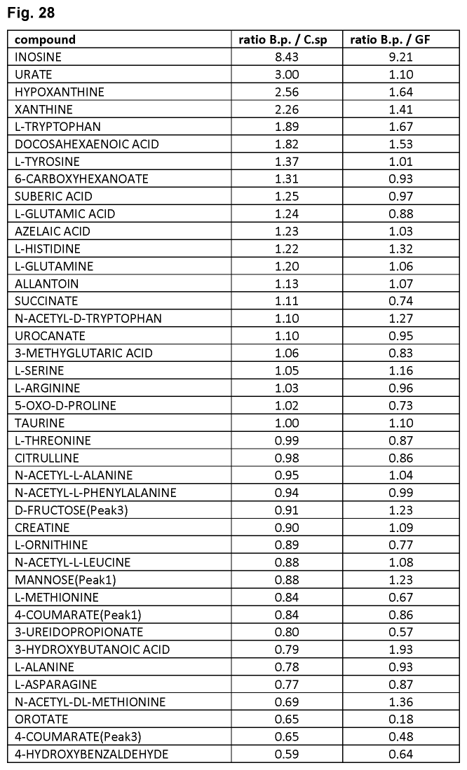

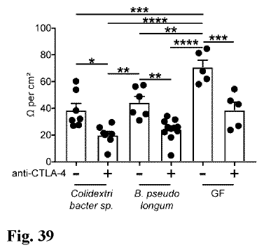

In order to identify putative metabolites that might be responsible for the anti-tumor effects of the transferred serum, we determined the metabolomic profile of the transferred serum samples and identified metabolites that were increased in the serum of mice monocolonized with B.p. compared to C.sp. or GF mice. Untargeted metabolomics analysis revealed increased levels of several metabolites in sera from B.p. compared to C.sp. monocolonized and GF mice (FIG. 4a and FIG. 15a, 15b). Notably, the purine metabolite inosine was the only metabolite that was significantly more abundant (8 to 9-fold) in sera from B.p. monocolonized mice compared to sera from C.sp. monocolonized or GF mice (FIG. 4b). Of note, xanthine and hypoxanthine, degradation products of inosine, were also elevated in the sera of B.p. monocolonized mice (FIG. 28). Analysis of bacterial culture supernatant revealed that B.p. produced approximately ten-fold higher amounts of inosine than C.sp. cultured under the same culture conditions, revealing that inosine is a bacterial metabolite produced by B.p. (FIG. 15c; FIG. 32). The identity of inosine was confirmed by fragmentation analysis (FIG. 15d). To determine physiological inosine levels in vivo we next measured inosine concentrations in duodenal, jejunal and cecal contents of B.p. monocolonized mice. Inosine concentrations were highest in the duodenum and gradually decreased along the gastrointestinal tract (duodenum 66.13±14.23 μM>jejunum 29.26±9.38 μM>cecum 0.5±0.05 μM; FIG. 15e; FIG. 38). We also quantified inosine concentrations in the serum of B.p. (26.16±3.32 μM) and C.sp. (3.26±1.01 μM) monocolonized mice (FIG. 15e), verifying our previous results. Furthermore, inosine levels in the serum of SPF mice (4.08±1.12 μM) increased significantly following anti-CTLA-4 treatment (11.65±2.09 μM) and this was greatly diminished in antibiotic-treated SPF mice (2.03±0.86 μM) (FIG. 15f). These data indicated that bacterial production in the upper gastrointestinal tract is likely to be the predominant source of systemic inosine in B.p. monocolonized mice.

We next investigated whether inosine could directly enhance anti-tumor Th1 cell differentiation. To test this, we co-cultured activated OVA₃₂₃₋₃₃₉ peptide-pulsed bone marrow derived dendritic cells (BMDCs) with naïve OVA₃₂₃₋₃₃₉-specific OT-II CD4⁺ T cells in the presence or absence of inosine. Intriguingly, inosine led to context-dependent induction or inhibition of CD4⁺ T cell differentiation. Specifically, in the presence of exogenous IFN-γ, inosine strongly boosted Th1 differentiation of naïve T cells (FIG. 4c) whereas in the absence of IFN-γ, inosine reduced Th1 induction (FIG. 4d and FIG. 16a). We then dissected the molecular mechanism through which inosine enhanced Th1 differentiation and found that addition of ZM241385, a pharmacological inhibitor of adenosine A_{2A} receptor (A_{2A}R) signaling, completely abrogated the effect of inosine (FIG. 4c). Moreover, addition of cell permeable cyclic AMP (db-CAMP), a signaling molecule downstream of A_{2A}R, restored Th1 differentiation and bypassed the need for inosine. In addition, inhibition of protein kinase A (PKA), a downstream effector molecule of CAMP, similarly negated inosine-driven Th1 differentiation (FIG. 4c). Lastly, the inosine-A2_{A}R-CAMP-PKA signaling cascade led to phosphorylation of the transcription factor cAMP response element-binding protein (CREB) (FIG. 4e), a known transcriptional enhancer of key Th1 differentiation factors such as IL-12 receptor and IFN-γ (Samten et al., 2005; Samten et al., 2008; Yao et al., 2013). Indeed, inosine-dependent upregulation of IL12Rβ2 was also observed (FIG. 16b). The effect of inosine was T cell intrinsic and was not mediated indirectly through DCs because the addition of inosine to naïve T cells that had been activated with anti-CD3/anti-CD28-coated beads also enhanced Th1 differentiation, even in the absence of IFN-γ (FIG. 16c). Furthermore, induction of Th1 differentiation and phosphorylation of CREB was absent when A2_{A}R-deficient T cells were stimulated with inosine (FIG. 16d, 16e). In contrast, bypassing the need for A2_{A}R signaling by using db-cAMP increased Th1 differentiation and phosphorylation of CREB in A2_{A}R-deficient T cells, confirming that the Th1 promoting effect of inosine is depended on A2_{A}R signaling (FIG. 16d, 16e). In addition, since pCREB is known to bind to key Th1 target genes, we also confirmed that inosine stimulation led to a sustained upregulation of Il12rb2 and Ifng gene transcription in CD4 T cells (FIG. 16f, 16g). Importantly, inosine dose response experiments revealed that the physiological concentrations of inosine observed in sera of B.p. but not C.sp. monocolonized mice were sufficient to induce Th1 activation (FIG. 16h). Since adenosine also binds to the A2_{A}R we also measured adenosine levels and found extremely low levels in intestinal contents and, importantly, no differences in serum levels between B.p. and C.sp. monocolonized mice (FIG. 16i), indicating that adenosine could not be mediating the ICB-promoting effects of B.p. Furthermore, adenosine dose response experiments revealed that the levels of adenosine in the serum were insufficient to promote Th1 activation and effector function (FIG. 16j).

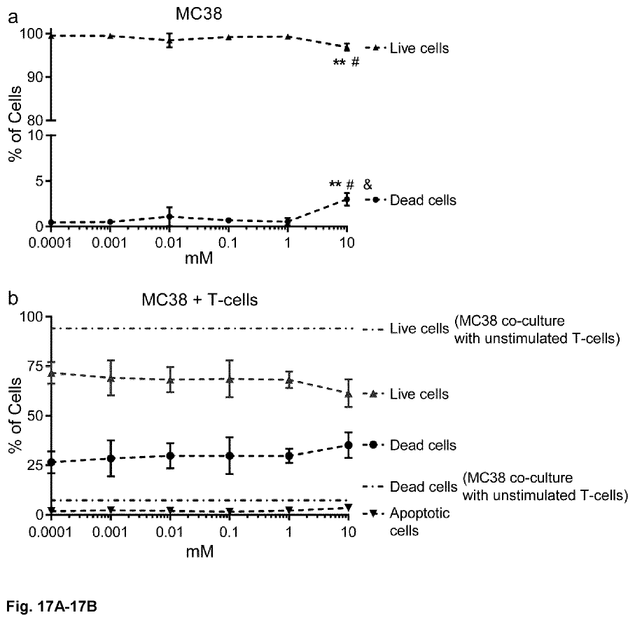

We next wondered if inosine could directly affect tumor cell survival or susceptibility to T cell-mediated killing. Direct exposure of MC38 tumor cells to inosine in vitro did not exert any effects on tumor cell viability (FIG. 17a). In addition, pretreatment of MC38 tumor cells prior to co-culture with activated tumor-specific T cells did not promote or inhibit T cell-mediated killing of tumor cells (FIG. 17b). These data indicate that the anti-tumor effect of inosine is mediated through T cells.

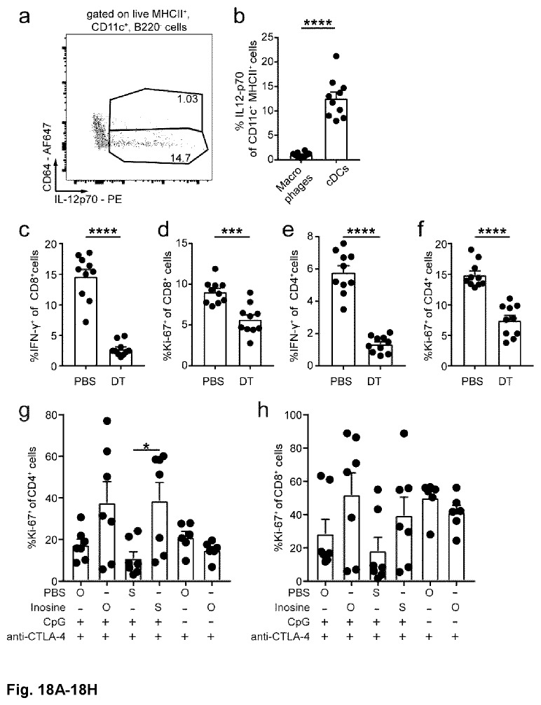

Combined, these data suggest that the effect of inosine on T cells required sufficient co-stimulation (likely by DCs), IL-12 receptor engagement for Th1 differentiation and IFN-γ production for efficient anti-tumor immunity. Classical dendritic cells (cDCs) were found to be the primary source of IL-12 compared to macrophages (FIGS. 18a and 18b). Thus, we evaluated the impact of cDCs during cancer and ICB-bacteria co-therapy. To do so, bone marrow (BM) cells from cDC-DTR mice were transferred into lethally γ-irradiated recipient (SPF) mice to allow for inducible, conditional depletion of cDCs. Following BM reconstitution, mice were treated with antibiotics and then gavaged with a mixture of the three previously identified ICB-promoting bacteria, B.p., L.j., and O.sp. Ten weeks after γ-irradiation, mice were subcutaneously injected with MC38 CRC cells and when palpable tumors were established, cDCs were depleted by injection of diphtheria toxin followed one day later by anti-CTLA-4 treatment (FIG. 4f). Depletion of cDCs led to a significant reduction in intratumoral CD8⁺ and CD4⁺ T cell frequencies and IFN-γ production (FIG. 4g-j), which resulted in larger tumors (FIG. 4k). Similarly, IFN-γ production and proliferation of splenic CD8⁺ and CD4⁺ T cells were also markedly reduced in cDC-depleted animals (FIG. 18c-18f). Therefore, depletion of cDC strongly reduced the efficacy of bacteria-elicited ICB to reduce established tumors, which indicates the requirement for continuous antigen presentation, IL-12 production and T cell co-stimulation by cDCs for efficient ICB therapy.

### Inosine Promotes Th1 Immunity and Tumoricidal Effects In Vivo

To confirm whether the inosine-mediated Th1 promoting effect in vitro also applied to in vivo conditions, GF mice were immunized with ovalbumin in combination with CpG as a co-stimulus. One day later mice received inosine or vehicle only intraperitoneally. Inosine increased the proportions of T-bet⁺, IFN-γ⁺ CD8⁺ and CD4⁺ T cells in the MLN (FIG. 5a-c), validating our in vitro results. In order to assess whether the effect of inosine also promoted anti-tumor immunity, we challenged GF mice with MC38 tumor cells. Upon palpable tumors, inosine or PBS was given orally or systemically in combination with anti-CTLA-4 treatment and CpG as indicated (FIG. 5 d). Compared to PBS, inosine led to a reduction of tumor weight and increased anti-tumor immunity irrespective of oral or systemic application routes when given together with anti-CTLA-4 and CpG (FIG. 5e-g and FIG. 18g, 18h). In contrast, in the absence of CpG as a co-stimulus, inosine increased tumor weight and reduced anti-tumor immunity (FIG. 5 e-g and FIG. 18g, 18h). These results validate our previous in vitro findings demonstrating that the effect of inosine is context dependent based on the amount of co-stimulation present.

We then confirmed that inosine-induced anti-tumor immunity was dependent on A2_{A}R signaling in vivo. Germ-free Rag1-deficient animals were challenged with MC38 tumor cells and simultaneously received WT or A2_{A}R-deficient T cells. Seven days later, inosine was given orally in combination with anti-CTLA-4 and CpG (FIG. 5h). Inosine enhanced anti-CTLA-4/CpG-mediated anti-tumor immunity in animals that received WT but not A2AR-deficient T cells (FIG. 5i-l). Specifically, in the presence of inosine, WT but not A2AR-deficient T cells displayed increased IFN-γ production within tumors and subsequently only GF mice receiving WT T cells showed reduced tumor weights (FIG. 5i-l). This demonstrated a dependency on A2_{A}R signaling specifically in T cells for the anti-tumor effect of inosine-ICB co-therapy.

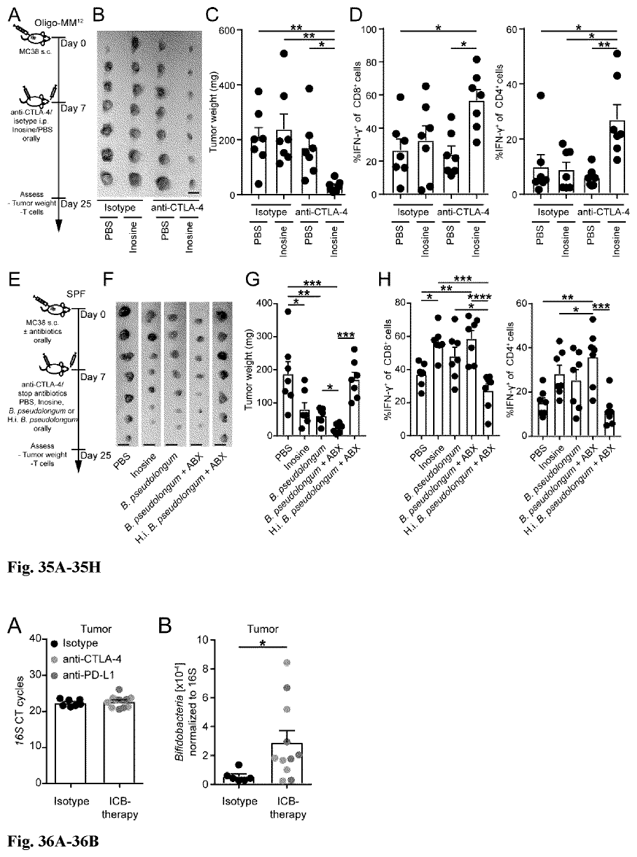

Since we detected A. muciniphila in ICB-treated tumors, that was previously shown to increase ICB therapy efficacy and to produce inosine in vitro, we further investigated whether A. muciniphila also relies on A2_{A}R signaling to enhance ICB-therapy efficacy. We found that monocolonization with A. muciniphila in combination with anti-CTLA-4 led to smaller tumors and increased anti-tumor immunity and this was dependent on T cell expression of A2_{A}R (FIG. 34A-34D). Although monocolonization with L. johnsonii was able promote the anti-tumor effects of anti-CTLA-4, hypoxanthine (another ligand of the A2_{A}R), and not inosine, was elevated in in vitro cultures. Despite this, the ICB-promoting effect of L. johnsonii, although less potent than that of B. pseudolongum and A. muciniphila, was also partially dependent on T cell expression of A2_{A}R (FIG. 34E-34H).

We next tested whether inosine could also promote the efficacy of anti-CTLA-4 therapy in the presence of a complex microbiota. We first utilized a gnotobiotic model where mice are stably colonized with a defined microbiota consisting of 12 bacterial species, referred to as Oligo-Mouse-Microbiota-12 (Oligo-MM12), which lacks B. pseudolongum. We found that inosine was able to promote the anti-tumor effects of anti-CTLA-4 with reduced tumor size and increased intra-tumoral IFN-γ+CD8+ and IFN-y+CD4+ T cells even in gnotobiotic Oligo-MM12 mice (FIG. 35A-35D). We also found that inosine could promote the efficacy of anti-CTLA-4 in SPF mice, that contain a highly diverse microbiota (FIG. 35E-35H). We then examined whether B. pseudolongum needed to be viable to enhance anti-CTLA-4 efficacy. While gavage of live B. pseudolongum, with or without antibiotic pretreatment, enhanced anti-CTLA-4 effects in SPF mice, heat-killed B. pseudolongum was unable to boost the effects of ICB therapy, likely due to the inability to produce inosine (FIG. 35E to 35H).

Taken together, inosine-A2_{A}R signaling drives or inhibits anti-tumor immunity in vivo, depending on the amount of co-stimulation present.

### Differential Effect of ICB-Promoting Bacteria on CRC Subtypes

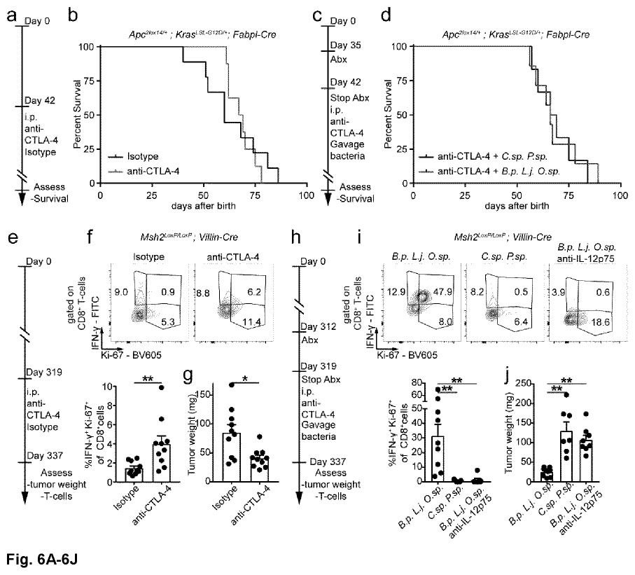

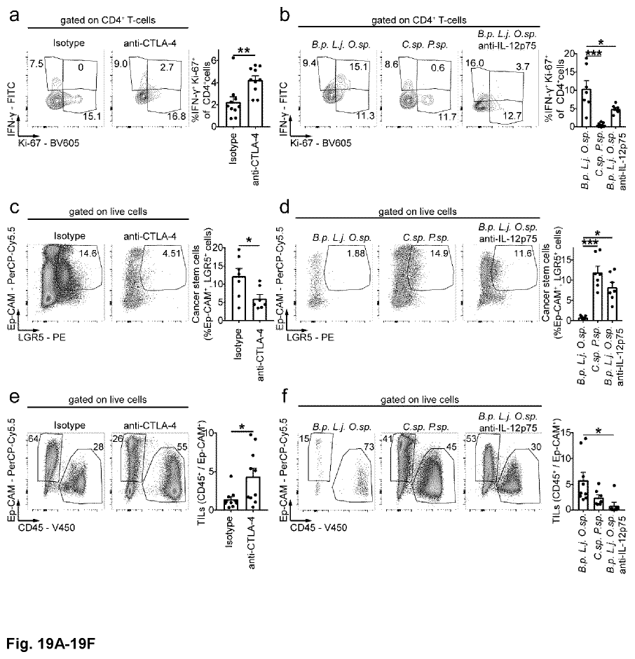

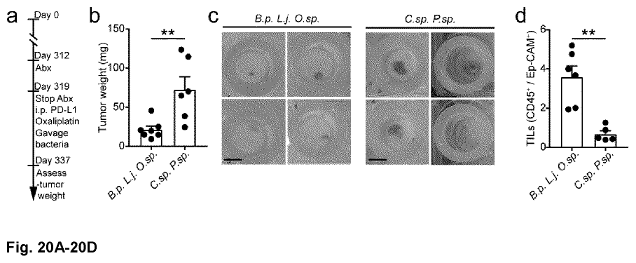

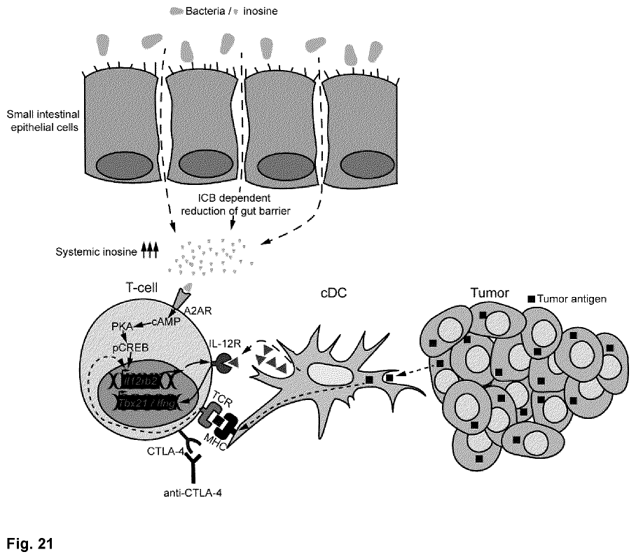

Lastly, we examined the effect of the identified ICB-promoting bacteria in two distinct models of CRC that mimic different subtypes of human CRC. First, we tested the ICB-promoting effect of B.p., L.j., and O.sp. in Apc^{2lox14/+}; Kras^{LSL-G12D/+}; Fabpl-Cre (Haigis et al., 2008) SPF mice, which have conditional Apc deficiency and activation of Kras specifically in colonocytes. In this model of CRC, anti-CTLA-4 treatment alone did not improve survival compared to isotype-treated animals (FIG. 6a, b). To test if the addition of ICB-promoting bacteria could switch a non-responsive to a responsive effect in this model, SPF mice were treated with a mixture of broad-spectrum antibiotics for 7 days to overcome colonization resistance (Lee et al., 2013), followed by bacterial transfer and treatment with anti-CTLA-4. Although the ICB-promoting bacteria colonized the intestine, this combined approach did not enhance survival (FIG. 6c, d), revealing a limitation of bacterial co-therapy in this model. Next, we examined the effect of ICB-promoting bacteria in SPF Msh2^{LoxP/LoxP} Villin-Cre (Kucherlapati et al., 2010) animals that have conditional inactivation of Msh2 in intestinal epithelial cells. In this model, anti-CTLA-4 treatment alone (without the addition of ICB-promoting bacteria) led to reduced tumor weight and cancer stem cells and increased T cell activation and immune cell infiltration in the tumor (FIG. 6e-g, FIGS. 19a, 19c, and 19e). Remarkably, co-treatment with ICB-promoting bacteria boosted the effect of anti-CTLA-4, leading to a further marked reduction of tumor weight and cancer stem cell numbers together with drastically enhanced T cell activation and immune cell infiltration in the tumor compared to control bacteria (FIG. 6h-j, FIGS. 19b, 19d, and 19f). In support of a critical role for inosine-dependent upregulation of IL12Rβ2 on T cells and cDC IL-12 production and function, anti-IL-12p75 treatment almost completely abrogated the effect of ICB-promoting, anti-CTLA-4 co-therapy in Msh2^{LoxP/LoxP} Villin-Cre tumors, which corroborates similar findings upon simultaneous depletion of IL-12 and IL-23, using anti-IL-12p40 treatment (Routy et al., 2018; Vetizou et al., 2015). Finally, since oxaliplatin-anti-PD-L1 co-treatment is a more commonly used therapy in the clinics, we confirmed that ICB-promoting bacteria also promoted the efficacy of oxaliplatin-anti-PD-L1 treatment in SPF Msh2^{LoxP/LoxP} Villin-Cre (Kucherlapati et al., 2010) animals (FIG. 20).

As B. pseudolongum was enriched in AOM/DSS tumors of ICB-treated animals and Bifidobacteria were previously associated with improved ICB-therapy efficacy in cancer patients, we wondered whether Bifidobacteria were also enriched in Msh2^{LoxP/LoxP} Villin-Cre tumors of ICB-treated mice. While the total amount of tumor-associated bacteria did not change with anti-CTLA-4 or anti-PD-L1 treatment (FIG. 36A), ICB treatment led to specific enrichment of tumor-associated Bifidobacteria (FIG. 36B).

In summary, our results reveal a novel bacterial-inosine-immune pathway that boosts a cDC-dependent Th1 T cell circuit to greatly enhance the effect of ICB therapies in CRC (FIG. 21).

### Effects of Additional Bifidobacterium Species on ICB Therapy

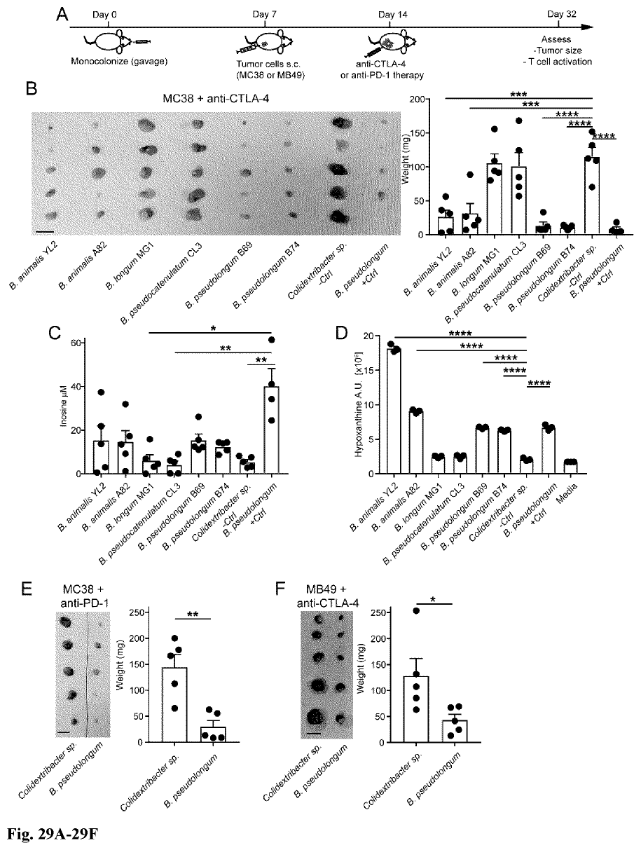

Next, we investigated whether ICB-promoting properties were a conserved feature among multiple Bifidobacterium species. To test this, we monocolonized GF mice with different Bifidobacterium species, challenged these mice with MC38 colorectal cancer cells and treated them with anti-CTLA-4. Intriguingly, some but not all Bifidobacterium species tested improved ICB therapy efficacy (FIGS. 29A and B). All three bacterial strains belonging to Bifidobacterium pseudolongum species had the greater ICB-promoting effect among other species of Bifidobacterium tested. These results show that the ICB-promoting effects were species dependent. Consistent with our findings, ICB-improving Bifidobacterium species had elevated serum inosine levels (FIG. 29C) and the corresponding bacteria produced increased amounts of the A2_{A} ligand hypoxanthine (degradation product of inosine) in vitro, whereas Bifidobacteria that did not improve ICB-therapy did not produce hypoxanthine (FIG. 29 D).

Moreover, we also tested if the Bifidobacterium pseudolongum strain isolated by us improved the efficacy of another ICB, anti-PD-1. Indeed, compared to our control bacterium (Colidextribacter sp.), B. pseudolongum together with anti-PD-1 improved anti-tumor immunity against MC38 tumor cells (FIG. 29 E). Lastly, B. pseudolongum compared to Colidextribacter sp. also increased the efficacy of anti-CTLA-4 against a bladder cancer cell line (MB49) (FIG. 29F). This indicates that B. pseudolongum increased ICB-efficacy in different tumor types.

### Discussion

ICB therapy has yielded rather disappointing results in CRC, with an objective response only in 40% of patients with the mismatch repair deficient (MMRD) sub-type of CRC, which amounts to only 4% of all CRC (Le et al., 2015). We have now identified a novel microbial-metabolite-immune circuit that enhances ICB therapy in two mouse models of CRC. These data indicate that modification of the microbiota may provide a promising adjuvant therapy to ICB in CRC. Of note, compared to anti-PD-L1, anti-CTLA-4 induced stronger anti-tumor effects in the AOM/DSS and heterotopic tumor models when both antibodies were administered at the same dose. At this point it is difficult to know if this is due to differences in the biological effects of blocking CTLA-4 versus PD-L1 in these models, but it should be noted that other experimental studies routinely use anti-PD-1 mAb at much higher does than anti-CTLA4 mAb (Routy et al., 2018).

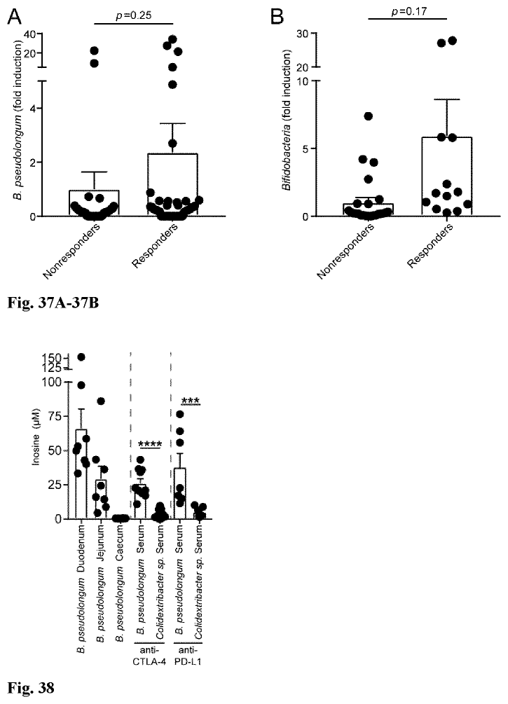

By isolating tumor-associated bacteria we have identified several bacterial species that were found to be associated exclusively with tumors following treatment with ICB, with three of these bacteria able to significantly enhance the efficacy of ICB therapy in CRC. This suggests that the isolation of bacterial species from intestinal tumor biopsies rather than from feces may be a better approach in a clinical setting for defining ICB-promoting bacteria in CRC. Although isolated from mice, all three ICB-promoting bacteria are also found in humans, indicating their potential for clinical translation (Dewhirst et al., 2001; Pridmore et al., 2008; Turroni et al., 2009). Furthermore, we analyzed published human fecal microbiome metagenomic datasets and found a trend, although not significant, where B. pseudolongum was enriched [up to 2.4-fold] in responders compared to nonresponding cancer patients (FIG. 37A). At the genus level, Bifidobacteria were also enriched (albeit non-significantly) in responders versus nonresponders [5.9-fold; FIG. 37B], with the species B. longum and B. adolescentis significantly enriched. Although Bifidobacterium species, such as B. breve and B. longum, have previously been associated with anti-tumor immunity (Sivan et al., 2015), other Bifidobacterium species have been reported to provide protection from anti-CTLA-4-induced enterocolitis with no effect on tumor growth (Wang et al., 2018). Bifidobacterium pseudolongum species are widely distributed in the mammalian gut with many different strains displaying genomic diversity and differential metabolic capacities (Lugli et al., 2019), suggesting strain-dependent functions and a need for a precision approach to microbial therapy. Lactobacillus johnsonii has not previously been associated with anti-tumor immunity, in contrast, it has been shown to have anti-inflammatory effects (Bereswill et al., 2017). Much less is known about the functions of Olsenella species.

Our findings demonstrate a critical role for the bacterial metabolite inosine in setting a baseline Th1 level in local mucosal tissues. Initially, this was surprising because previous reports have demonstrated an inhibitory effect of inosine, and A2_{A}R engagement in general, on Th1 differentiation in vitro and anti-tumor immunity in vivo (Csoka et al., 2008; Hasko et al., 2000; He et al., 2017; Ohta et al., 2006). Indeed, the wealth of data supporting an immunosuppressive role for adenosine and A2_{A}R signaling has led to the development of novel immune checkpoint inhibitor targets, such as mAb targeting CD73, CD39 and CD38, and pharmacological antagonists of A2_{A}R, many of which are currently in clinical trials (reviewed in (Vigano et al., 2019)). However, a small body of literature has demonstrated that inosine can be pro-inflammatory and A2_{A}R signaling can sustain Th1/anti-tumor immunity in mice (Cekic and Linden, 2014; Lasek et al., 2015; Lioux et al., 2016). Our findings reconcile these contrasting observations by revealing a context-dependent effect of inosine-A2_{A} receptor signaling based on the amount of co-stimulation. Mechanistically, inosine engages the A2_{A} receptor and activates the transcription factor CREB, through cAMP. CREB, together with co-factors and the formation of heterodimers with ATF-2 and/or c-Jun, modulates the transcription of key Th1 genes, including Il2rb2 and Ifng (Samten et al., 2008). It is worth noting that in addition to CAMP signaling, inosine (compared to adenosine) has a distinct A2_{A}R-dependent signaling bias, with a 3.3-fold preference for ERK1/2 phosphorylation. In light of our findings, blockade of inosine-A2_{A} receptor signaling in cancer immunotherapy could negate a positive effect provided by beneficial microbes. We suggest that A2_{A} receptor signaling is likely an integral anti-tumor pathway for bacterial-ICB co-therapies. Indeed, Tanoue et al recently identified a consortium of eleven bacteria that improve ICB therapies (Tanoue et al., 2019), which are not related to the bacteria identified in this work. Remarkably though, two of the most elevated metabolites in the cecum and serum of mice colonized with the consortium of 11 bacteria were inosine monophosphate and hypoxanthine, a substrate and product of inosine respectively, which are both A2_{A} receptor agonists like inosine (Welihinda et al., 2016). The identification of this context-dependent effect of inosine-A2_{A} receptor signaling is particularly relevant as inosine is currently used as an intervention in clinical trials in various Th1-associated diseases (Clinical.trials.gov), including multiple sclerosis, amyotrophic lateral sclerosis and Parkinson's disease (Bettelli et al., 2004; Kustrimovic et al., 2018; Lovett-Racke et al., 2004; Saresella et al., 2013).

We identified cDCs and their production of IL-12 as essential components for efficient induction of anti-tumor T cell immunity elicited by ICB therapy in the presence of beneficial bacteria. The critical involvement of cDC and IL-12 has also been recently reported upon anti-PD-1 treatment (Garris et al., 2018).

Seminal work by Guinney et. al revealed four molecular consensus subtypes of CRC (Guinney et al., 2015); MMRD, canonical, metabolic and mesenchymal. In line with the positive results of ICB in MMRD patients in the clinical setting (Le et al., 2015), in our animal model of MMRD (Msh2^{LoxP/LoxP} Villin-Cre) we indeed observed some efficacy of anti-CTLA-4 single therapy. However, co-therapy with ICB-promoting bacteria strongly enhanced the tumoricidal effect of anti-CTLA-4. Thus, bacterial co-therapy may optimize treatment regimens in MMRD CRC patients. Secondly, ICB therapy was efficacious and was associated with B. pseudolongum, L. johnsonii, and Olsenella sp. in the AOM/DSS model of CRC. AOM/DSS tumors have been used to model inflammation-associated CRC. AOM/DSS tumors also display characteristics of epithelial to mesenchymal transition (Lin et al., 2015), such as reduced E-Cadherin, increased N-Cadherin, Vimentin and SNAIL expression as well as inflammation and increased TGF-β expression (Becker et al., 2004; Mager et al., 2017), which are hallmarks of the mesenchymal consensus molecular CRC subtype (Guinney et al., 2015). Thus, our results indicate a benefit of bacterial co-therapy also in this subtype. Canonical and metabolic CRC subtypes are both characterized by inactivation of Apc, canonical additionally by Wnt pathway and metabolic by KRAS activation (Guinney et al., 2015). These hallmarks are well represented in the Apc^{2lox14/+}; Kras^{LSL-G12D/+}; Fabpl-Cre animal model and intriguingly bacterial co-therapy did not improve anti-CTLA-4 treatment. The divergent effect of ICB-promoting bacteria in the Msh2^{LoxP/LoxP} Villin-Cre- compared to the Apc^{2lox14/+}; Kras^{LSL-G12D/+}; Fabpl-Cre model is intriguing and at this stage we can only speculate about the underlying reason(s). The mutational load and associated number of neoantigens, which is likely higher in Msh2^{LoxP/LoxP} Villin-Cre tumors, certainly impacts on the efficacy of ICB therapies (Havel et al., 2019). Moreover, anti-CTLA-4 had no effect on its own in the Apc^{2lox14/+}; Kras^{LSL-G12D/+}; Fabpl-Cre model and bacteria alone did not impact on heterotopic tumor development. We also showed that B.p. increased the Th1 cell pool and their anti-tumor effect was unleashed followed by effective ICB therapy. Thus, we reason that the discovery of novel checkpoint blockade targets or other therapies that have an effect of their own in the Apc^{2lox14/+}; Kras^{LSL-G12D/+}; Fabpl-Cre model are required to enable efficacious bacterial co-therapy to treat similar subtypes in CRC patients.

Together, this work paves the way for new approaches to treatment of cancers including CRC.

### REFERENCES

Allard, B., Cousineau, I., Allard, D., Buisseret, L., Pommey, S., Chrobak, P., and Stagg, J. (2019). Adenosine A2a receptor promotes lymphangiogenesis and lymph node metastasis. Oncoimmunology 8, 1601481.
Anitei, M. G., Zeitoun, G., Mlecnik, B., Marliot, F., Haicheur, N., Todosi, A. M., Kirilovsky, A., Lagorce, C., Bindea, G., Ferariu, D., et al. (2014). Prognostic and predictive values of the immunoscore in patients with rectal cancer. Clin Cancer Res 20, 1891-1899.
Arthur, J. C., Perez-Chanona, E., Muhlbauer, M., Tomkovich, S., Uronis, J. M., Fan, T. J., Campbell, B. J., Abujamel, T., Dogan, B., Rogers, A. B., et al. (2012). Intestinal inflammation targets cancer-inducing activity of the microbiota. Science 338, 120-123.
Barnden, M. J., Allison, J., Heath, W. R., and Carbone, F. R. (1998). Defective TCR expression in transgenic mice constructed using cDNA-based alpha- and beta-chain genes under the control of heterologous regulatory elements. Immunol Cell Biol 76, 34-40.
Becker, C., Fantini, M. C., Schramm, C., Lehr, H. A., Wirtz, S., Nikolaev, A., Burg, J., Strand, S., Kiesslich, R., Huber, S., et al. (2004). TGF-beta suppresses tumor progression in colon cancer by inhibition of IL-6 trans-signaling. Immunity 21, 491-501.
Bereswill, S., Ekmekciu, I., Escher, U., Fiebiger, U., Stingl, K., and Heimesaat, M. M. (2017). Lactobacillus johnsonii ameliorates intestinal, extra-intestinal and systemic pro-inflammatory immune responses following murine Campylobacter jejuni infection. Sci Rep 7, 2138.
Bettelli, E., Sullivan, B., Szabo, S. J., Sobel, R. A., Glimcher, L. H., and Kuchroo, V. K. (2004). Loss of T-bet, but not STAT1, prevents the development of experimental autoimmune encephalomyelitis. J Exp Med 200, 79-87.
Brahmer, J. R., Tykodi, S. S., Chow, L. Q., Hwu, W. J., Topalian, S. L., Hwu, P., Drake, C. G., Camacho, L. H., Kauh, J., Odunsi, K., et al. (2012). Safety and activity of anti-PD-L1 antibody in patients with advanced cancer. N Engl J Med 366, 2455-2465.
Callahan, B. J., McMurdie, P. J., Rosen, M. J., Han, A. W., Johnson, A. J., and Holmes, S P. (2016). DADA2: High-resolution sample inference from Illumina amplicon data. Nat Methods 13, 581-583.
Cekic, C., and Linden, J. (2014). Adenosine A2A receptors intrinsically regulate CD8⁺ T cells in the tumor microenvironment. Cancer Res 74, 7239-7249.
Clasquin, M. F., Melamud, E., and Rabinowitz, J. D. (2012). LC-MS data processing with MAVEN: a metabolomic analysis and visualization engine. Curr Protoc Bioinformatics Chapter 14, Unit 14 11.
Clinical.trials.gov at. https://clinicaltrialsgov/ct2/results?intr-inosine&Search=Apply&age_v=&gndr-&type=& rslt=.
Csoka, B., Himer, L., Selmeczy, Z., Vizi, E. S., Pacher, P., Ledent, C., Deitch, E. A., Spolarics, Z., Nemeth, Z. H., and Hasko, G. (2008). Adenosine A2A receptor activation inhibits T helper 1 and T helper 2 cell development and effector function. FASEB J 22, 3491-3499.
Dejea, C. M., Fathi, P., Craig, J. M., Boleij, A., Taddese, R., Geis, A. L., Wu, X., DeStefano Shields, C. E., Hechenbleikner, E. M., Huso, D. L., et al. (2018). Patients with familial adenomatous polyposis harbor colonic biofilms containing tumorigenic bacteria. Science 359, 592-597.
DeSantis, T. Z., Hugenholtz, P., Larsen, N., Rojas, M., Brodie, E. L., Keller, K., Huber, T., Dalevi, D., Hu, P., and Andersen, G. L. (2006). Greengenes, a chimera-checked 16S rRNA gene database and workbench compatible with ARB. Appl Environ Microbiol 72, 5069-5072.
Dewhirst, F. E., Paster, B. J., Tzellas, N., Coleman, B., Downes, J., Spratt, D. A., and Wade, W. G. (2001). Characterization of novel human oral isolates and cloned 16S rDNA sequences that fall in the family Coriobacteriaceae: description of olsenella gen. nov., reclassification of Lactobacillus uli as Olsenella uli comb. nov. and description of Olsenella profusa sp. nov. Int J Syst Evol Microbiol 51, 1797-1804.
Ferlay, J., Soerjomataram, I., Dikshit, R., Eser, S., Mathers, C., Rebelo, M., Parkin, D. M., Forman, D., and Bray, F. (2015). Cancer incidence and mortality worldwide: sources, methods and major patterns in GLOBOCAN 2012. Int J Cancer 136, E359-386.
Galon, J., Costes, A., Sanchez-Cabo, F., Kirilovsky, A., Mlecnik, B., Lagorce-Pages, C., Tosolini, M., Camus, M., Berger, A., Wind, P., et al. (2006). Type, density, and location of immune cells within human colorectal tumors predict clinical outcome. Science 313, 1960-1964.
Garris, C. S., Arlauckas, S P., Kohler, R. H., Trefny, M. P., Garren, S., Piot, C., Engblom, C., Pfirschke, C., Siwicki, M., Gungabeesoon, J., et al. (2018). Successful Anti-PD-1 Cancer Immunotherapy Requires T Cell-Dendritic Cell Crosstalk Involving the Cytokines IFN-gamma and IL-12. Immunity 49, 1148-1161 e1147.
Germann, T., Bongartz, M., Dlugonska, H., Hess, H., Schmitt, E., Kolbe, L., Kolsch, E., Podlaski, F. J., Gately, M. K., and Rude, E. (1995). Interleukin-12 profoundly up-regulates the synthesis of antigen-specific complement-fixing IgG2a, IgG2b and IgG3 antibody subclasses in vivo. Eur J Immunol 25, 823-829.
Gowda, H., Ivanisevic, J., Johnson, C. H., Kurczy, M. E., Benton, H. P., Rinehart, D., Nguyen, T., Ray, J., Kuehl, J., Arevalo, B., et al. (2014). Interactive XCMS Online: simplifying advanced metabolomic data processing and subsequent statistical analyses. Anal Chem 86, 6931-6939.
Grivennikov, S. I., Wang, K., Mucida, D., Stewart, C. A., Schnabl, B., Jauch, D., Taniguchi, K., Yu, G. Y., Osterreicher, C. H., Hung, K. E., et al. (2012). Adenoma-linked barrier defects and microbial products drive IL-23/IL-17-mediated tumour growth. Nature 491, 254-258.
Guinney, J., Dienstmann, R., Wang, X., de Reynies, A., Schlicker, A., Soneson, C., Marisa, L., Roepman, P., Nyamundanda, G., Angelino, P., et al. (2015). The consensus molecular subtypes of colorectal cancer. Nat Med 21, 1350-1356.
Haigis, K. M., Kendall, K. R., Wang, Y., Cheung, A., Haigis, M. C., Glickman, J. N., Niwa-Kawakita, M., Sweet-Cordero, A., Sebolt-Leopold, J., Shannon, K. M., et al. (2008). Differential effects of oncogenic K-Ras and N-Ras on proliferation, differentiation and tumor progression in the colon. Nat Genet 40, 600-608.
Hasko, G., Kuhel, D. G., Nemeth, Z. H., Mabley, J. G., Stachlewitz, R. F., Virag, L., Lohinai, Z., Southan, G. J., Salzman, A. L., and Szabo, C. (2000). Inosine inhibits inflammatory cytokine production by a posttranscriptional mechanism and protects against endotoxin-induced shock. J Immunol 164, 1013-1019.
Havel, J. J., Chowell, D., and Chan, T. A. (2019). The evolving landscape of biomarkers for checkpoint inhibitor immunotherapy. Nat Rev Cancer 19, 133-150.
He, B., Hoang, T. K., Wang, T., Ferris, M., Taylor, C. M., Tian, X., Luo, M., Tran, D. Q., Zhou, J., Tatevian, N., et al. (2017). Resetting microbiota by Lactobacillus reuteri inhibits T reg deficiency-induced autoimmunity via adenosine A2A receptors. J Exp Med 214, 107-123.
Hodi, F. S., O'Day, S. J., McDermott, D. F., Weber, R. W., Sosman, J. A., Haanen, J. B., Gonzalez, R., Robert, C., Schadendorf, D., Hassel, J. C., et al. (2010). Improved survival with ipilimumab in patients with metastatic melanoma. N Engl J Med 363, 711-723.
Hogquist, K. A., Jameson, S. C., Heath, W. R., Howard, J. L., Bevan, M. J., and Carbone, F. R. (1994). T cell receptor antagonist peptides induce positive selection. Cell 76, 17-27.
Iida, N., Dzutsev, A., Stewart, C. A., Smith, L., Bouladoux, N., Weingarten, R. A., Molina, D. A., Salcedo, R., Back, T., Cramer, S., et al. (2013). Commensal bacteria control cancer response to therapy by modulating the tumor microenvironment. Science 342, 967-970.
Kozich, J. J., Westcott, S. L., Baxter, N. T., Highlander, S. K., and Schloss, P. D. (2013). Development of a dual-index sequencing strategy and curation pipeline for analyzing amplicon sequence data on the MiSeq Illumina sequencing platform. Appl Environ Microbiol 79, 5112-5120.
Kucherlapati, M. H., Lee, K., Nguyen, A. A., Clark, A. B., Hou, H., Jr., Rosulek, A., Li, H., Yang, K., Fan, K., Lipkin, M., et al. (2010). An Msh2 conditional knockout mouse for studying intestinal cancer and testing anticancer agents. Gastroenterology 138, 993-1002 e1001.
Kustrimovic, N., Comi, C., Magistrelli, L., Rasini, E., Legnaro, M., Bombelli, R., Aleksic, I., Blandini, F., Minafra, B., Riboldazzi, G., et al. (2018). Parkinson's disease patients have a complex phenotypic and functional Th1 bias: cross-sectional studies of CD4+ Th1/Th2/T17 and Treg in drug-naive and drug-treated patients. J Neuroinflammation 15, 205.
Lasek, W., Janyst, M., Wolny, R., Zapala, L., Bocian, K., and Drela, N. (2015). Immunomodulatory effects of inosine pranobex on cytokine production by human lymphocytes. Acta Pharm 65, 171-180.
Le, D. T., Durham, J. N., Smith, K. N., Wang, H., Bartlett, B. R., Aulakh, L. K., Lu, S., Kemberling, H., Wilt, C., Luber, B. S., et al. (2017). Mismatch repair deficiency predicts response of solid tumors to PD-1 blockade. Science 357, 409-413.
Le, D. T., Uram, J. N., Wang, H., Bartlett, B. R., Kemberling, H., Eyring, A. D., Skora, A. D., Luber, B. S., Azad, N. S., Laheru, D., et al. (2015). PD-1 Blockade in Tumors with Mismatch-Repair Deficiency. N Engl J Med 372, 2509-2520.
Le Gouvello, S., Bastuji-Garin, S., Aloulou, N., Mansour, H., Chaumette, M. T., Berrehar, F., Seikour, A., Charachon, A., Karoui, M., Leroy, K., et al. (2008). High prevalence of Foxp3 and IL17 in MMR-proficient colorectal carcinomas. Gut 57, 772-779.
Lee, S. M., Donaldson, G. P., Mikulski, Z., Boyajian, S., Ley, K., and Mazmanian, S. K. (2013). Bacterial colonization factors control specificity and stability of the gut microbiota. Nature 501, 426-429.
Lin, L., Sun, Y., Wang, D., Zheng, S., Zhang, J., and Zheng, C. (2015). Celastrol Ameliorates Ulcerative Colitis-Related Colorectal Cancer in Mice via Suppressing Inflammatory Responses and Epithelial-Mesenchymal Transition. Front Pharmacol 6, 320.
Lioux, T., Mauny, M. A., Lamoureux, A., Bascoul, N., Hays, M., Vernejoul, F., Baudru, A. S., Boularan, C., Lopes-Vicente, J., Qushair, G., et al. (2016). Design, Synthesis, and Biological Evaluation of Novel Cyclic Adenosine-Inosine Monophosphate (cAIMP) Analogs That Activate Stimulator of Interferon Genes (STING). J Med Chem 59, 10253-10267.
Love, M. I., Huber, W., and Anders, S. (2014). Moderated estimation of fold change and dispersion for RNA-seq data with DESeq2. Genome Biol 15, 550.
Lovett-Racke, A. E., Rocchini, A. E., Choy, J., Northrop, S. C., Hussain, R. Z., Ratts, R. B., Sikder, D., and Racke, M. K. (2004). Silencing T-bet defines a critical role in the differentiation of autoreactive T lymphocytes. Immunity 21, 719-731.
Lugli, G. A., Duranti, S., Albert, K., Mancabelli, L., Napoli, S., Viappiani, A., Anzalone, R., Longhi, G., Milani, C., Turroni, F., et al. (2019). Unveiling genomic diversity among members of the Bifidobacterium pseudolongum species, a widely distributed gut commensal of the animal kingdom. Appl Environ Microbiol.
Mager, L. F., Koelzer, V. H., Stuber, R., Thoo, L., Keller, I., Koeck, I., Langenegger, M., Simillion, C., Pfister, S P., Faderl, M., et al. (2017). The ESRP1-GPR137 axis contributes to intestinal pathogenesis. Elife 6.
Mager, L. F., Riether, C., Schurch, C. M., Banz, Y., Wasmer, M. H., Stuber, R., Theocharides, A. P., Li, X., Xia, Y., Saito, H., et al. (2015). IL-33 signaling contributes to the pathogenesis of myeloproliferative neoplasms. J Clin Invest 125, 2579-2591.
Mager, L. F., Wasmer, M. H., Rau, T. T., and Krebs, P. (2016). Cytokine-Induced Modulation of Colorectal Cancer. Front Oncol 6, 96.
Melamud, E., Vastag, L., and Rabinowitz, J. D. (2010). Metabolomic analysis and visualization engine for LC-MS data. Anal Chem 82, 9818-9826.
Meredith, M. M., Liu, K., Darrasse-Jeze, G., Kamphorst, A. O., Schreiber, H. A., Guermonprez, P., Idoyaga, J., Cheong, C., Yao, K. H., Niec, R. E., et al. (2012). Expression of the zinc finger transcription factor zDC (Zbtb46, Btbd4) defines the classical dendritic cell lineage. J Exp Med 209, 1153-1165.
Mertz, K. D., Mager, L. F., Wasmer, M. H., Thiesler, T., Koelzer, V. H., Ruzzante, G., Joller, S., Murdoch, J. R., Brummendorf, T., Genitsch, V., et al. (2016). The IL-33/ST2 pathway contributes to intestinal tumorigenesis in humans and mice. Oncoimmunology 5, e1062966.
Mlecnik, B., Bindea, G., Angell, H. K., Maby, P., Angelova, M., Tougeron, D., Church, S. E., Lafontaine, L., Fischer, M., Fredriksen, T., et al. (2016). Integrative Analyses of Colorectal Cancer Show Immunoscore Is a Stronger Predictor of Patient Survival Than Microsatellite Instability. Immunity 44, 698-711.
Ohta, A., Gorelik, E., Prasad, S. J., Ronchese, F., Lukashev, D., Wong, M. K., Huang, X., Caldwell, S., Liu, K., Smith, P., et al. (2006). A2A adenosine receptor protects tumors from antitumor T cells. Proc Natl Acad Sci USA 103, 13132-13137.
Pridmore, R. D., Pittet, A. C., Praplan, F., and Cavadini, C. (2008). Hydrogen peroxide production by Lactobacillus johnsonii NCC 533 and its role in anti-Salmonella activity. FEMS Microbiol Lett 283, 210-215.
Routy, B., Le Chatelier, E., Derosa, L., Duong, C. P. M., Alou, M. T., Daillere, R., Fluckiger, A., Messaoudene, M., Rauber, C., Roberti, M. P., et al. (2018). Gut microbiome influences efficacy of PD-1-based immunotherapy against epithelial tumors. Science 359, 91-97.
Samten, B., Howard, S. T., Weis, S. E., Wu, S., Shams, H., Townsend, J. C., Safi, H., and Barnes, P. F. (2005). Cyclic AMP response element-binding protein positively regulates production of IFN-gamma by T cells in response to a microbial pathogen. J Immunol 174, 6357-6363.
Samten, B., Townsend, J. C., Weis, S. E., Bhoumik, A., Klucar, P., Shams, H., and Barnes, P. F. (2008). CREB, ATF, and AP-1 transcription factors regulate IFN-gamma secretion by human T cells in response to mycobacterial antigen. J Immunol 181, 2056-2064.
Saresella, M., Piancone, F., Tortorella, P., Marventano, I., Gatti, A., Caputo, D., Lunetta, C., Corbo, M., Rovaris, M., and Clerici, M. (2013). T helper-17 activation dominates the immunologic milieu of both amyotrophic lateral sclerosis and progressive multiple sclerosis. Clin Immunol 148, 79-88.
Schwabe, R. F., and Jobin, C. (2013). The microbiome and cancer. Nat Rev Cancer 13, 800-812.
Sivan, A., Corrales, L., Hubert, N., Williams, J. B., Aquino-Michaels, K., Earley, Z. M., Benyamin, F. W., Lei, Y. M., Jabri, B., Alegre, M. L., et al. (2015). Commensal Bifidobacterium promotes antitumor immunity and facilitates anti-PD-L1 efficacy. Science 350, 1084-1089.
Tanoue, T., Morita, S., Plichta, D. R., Skelly, A. N., Suda, W., Sugiura, Y., Narushima, S., Vlamakis, H., Motoo, I., Sugita, K., et al. (2019). A defined commensal consortium elicits CD8 T cells and anti-cancer immunity. Nature 565, 600-605.
Tautenhahn, R., Patti, G. J., Rinehart, D., and Siuzdak, G. (2012). XCMS Online: a web-based platform to process untargeted metabolomic data. Anal Chem 84, 5035-5039.
Turroni, F., Foroni, E., Pizzetti, P., Giubellini, V., Ribbera, A., Merusi, P., Cagnasso, P., Bizzarri, B., de'Angelis, G. L., Shanahan, F., et al. (2009). Exploring the diversity of the bifidobacterial population in the human intestinal tract. Appl Environ Microbiol 75, 1534-1545.
Vetizou, M., Pitt, J. M., Daillere, R., Lepage, P., Waldschmitt, N., Flament, C., Rusakiewicz, S., Routy, B., Roberti, M. P., Duong, C. P., et al. (2015). Anticancer immunotherapy by CTLA-4 blockade relies on the gut microbiota. Science 350, 1079-1084.
Viaud, S., Saccheri, F., Mignot, G., Yamazaki, T., Daillere, R., Hannani, D., Enot, D. P., Pfirschke, C., Engblom, C., Pittet, M. J., et al. (2013). The intestinal microbiota modulates the anticancer immune effects of cyclophosphamide. Science 342, 971-976.
Vigano, S., Alatzoglou, D., Irving, M., Menetrier-Caux, C., Caux, C., Romero, P., and Coukos, G. (2019). Targeting Adenosine in Cancer Immunotherapy to Enhance T-Cell Function. Front Immunol 10, 925.
Wang, F., Yin, Q., Chen, L., and Davis, M. M. (2018). Bifidobacterium can mitigate intestinal immunopathology in the context of CTLA-4 blockade. Proc Natl Acad Sci USA 115, 157-161.
Wang, L., Wang, Y., Song, Z., Chu, J., and Qu, X. (2015). Deficiency of interferon-gamma or its receptor promotes colorectal cancer development. J Interferon Cytokine Res 35, 273-280.
Welihinda, A. A., Kaur, M., Greene, K., Zhai, Y., and Amento, E. P. (2016). The adenosine metabolite inosine is a functional agonist of the adenosine A2A receptor with a unique signaling bias. Cell Signal 28, 552-560.
Yao, C., Hirata, T., Soontrapa, K., Ma, X., Takemori, H., and Narumiya, S. (2013). Prostaglandin E(2) promotes Th1 differentiation via synergistic amplification of IL-12 signalling by cAMP and PI3-kinase. Nat Commun 4, 1685.
Zheng, D. W., Chen, Y., Li, Z. H., Xu, L., Li, C. X., Li, B., Fan, J. X., Cheng, S. X., and Zhang, X. Z. (2018). Optically-controlled bacterial metabolite for cancer therapy. Nat Commun 9, 1680.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

### Exemplary Embodiments of the Invention

Exemplary aspects of the invention are specified by the following embodiments.
E1. A method of treating a subject having a cancer or suspected of having a cancer, comprising or consisting of, administering an immune checkpoint inhibitor and one or more bacterium selected from Bifidobacterium pseudolongum, Lactobacillus johnsonii, Olsenella profuse, Olsenella umbonata, or Olsenella uli.
E2. A method of treating a subject having a cancer or suspected of having a cancer, comprising or consisting of, administering an immune checkpoint inhibitor and one or more bacterium selected from Bifidobacterium pseudolongum, Lactobacillus johnsonii, or Olsenella sp.
E3. The method of embodiment E1 or E2, where the cancer is colorectal cancer (CRC), lung cancer, melanoma, bladder cancer, kidney cancer, breast cancer, prostate cancer, stomach cancer, liver cancer, esophageal cancer, pancreatic cancer, brain cancer, cervical cancer, ovarian cancer, thyroid cancer, lip cancer, oral cancer, larynx cancer, nasopharynx cancer, or uterine cancer.
E4. The method of embodiment E3, wherein the CRC is mismatch repair deficient (MMRD) CRC or inflammation-associated CRC.
E5. A method of treating a subject having or suspected of having colorectal cancer (CRC), comprising or consisting of, administering an immune checkpoint inhibitor and one or more bacterium selected from Bifidobacterium pseudolongum, Lactobacillus johnsonii, Olsenella profuse, Olsenella umbonata, or Olsenella uli.
E6. A method of treating a subject having or suspected of having colorectal cancer (CRC), comprising or consisting of, administering an immune checkpoint inhibitor and one or more bacteria selected from Bifidobacterium pseudolongum (B.p.), Lactobacillus johnsonii (L.j), or Olsenella sp. (O.sp.).
E7. A method of treating a subject having or suspected of having colorectal cancer (CRC), comprising or consisting of, administering an immune checkpoint inhibitor and one or more bacteria selected from Bifidobacterium sp. (B.sp.), Lactobacillus sp. (L.sp.), or Olsenella sp. (O.sp.).
E8. The method of any one of embodiments E5 to E7, wherein the CRC is mismatch repair deficient (MMRD) CRC or inflammation-associated CRC.
E9. The method of any one of embodiments E1 to E8, wherein said ICB inhibitor is an anti-CTLA4 antibody, or an anti-PD-L1 antibody, or an anti-PD-1 antibody.
E10. The method of any one of embodiments E1 to E9, wherein the Bifidobacterium sp. is presented in FIG. 22.
E11. The method of any one of embodiments E1 to E10, wherein the Lactobacillus sp. is presented in FIG. 23.
E12. The method of any one of embodiments E1 to E11, where the Olsenella sp. is presented in FIG. 24.
E13. The method of any one of embodiments E1 to E12, further comprising administration of a chemotherapeutic agent, an immunotherapeutic agent, or a radiotherapy.
E14. The method of any one of embodiments E1 to E13, wherein said subject is a human.
E15. Use of an immune checkpoint inhibitor and one or more bacterium selected from Bifidobacterium pseudolongum, Lactobacillus johnsonii, Olsenella profuse, Olsenella umbonata, or Olsenella uli, for treating a subject having a cancer or suspected of having a cancer.
E16. Use of an immune checkpoint inhibitor and one or more bacterium selected from Bifidobacterium pseudolongum, Lactobacillus johnsonii, or Olsenella sp. for treating a subject having a cancer or suspected of having a cancer.
E17. The use of embodiment E15 or E16, where the cancer is colorectal cancer (CRC), lung cancer, melanoma, bladder cancer, kidney cancer, breast cancer, prostate cancer, stomach cancer, liver cancer, esophageal cancer, pancreatic cancer, brain cancer, cervical cancer, ovarian cancer, thyroid cancer, lip cancer, oral cancer, larynx cancer, nasopharynx cancer, or uterine cancer.
E18. The use of embodiment E17, wherein the CRC is mismatch repair deficient (MMRD) CRC or inflammation-associated CRC.
E19. Use of an immune checkpoint inhibitor and one or more bacterium selected from Bifidobacterium pseudolongum, Lactobacillus johnsonii, Olsenella profuse, Olsenella umbonata, or Olsenella uli for treating a subject having or suspected of having colorectal cancer (CRC).
E20. Use of an immune checkpoint inhibitor and one or more bacteria selected from Bifidobacterium pseudolongum (B.p.), Lactobacillus johnsonii (L.j), or Olsenella sp. (O.sp.) for treating a subject having or suspected of having colorectal cancer (CRC).
E21. Use of an immune checkpoint inhibitor and one or more bacteria selected from Bifidobacterium sp. (B.sp.), Lactobacillus sp. (L.sp.), or Olsenella sp. (O.sp.) for treating a subject having or suspected of having colorectal cancer (CRC).
E22. The use of any one of embodiments E19 to E21, wherein the CRC is mismatch repair deficient (MMRD) CRC or inflammation-associated CRC.
E23. The use of any one of embodiments E15 to E22, wherein said ICB inhibitor is an anti-CTLA4 antibody, or an anti-PD-L1 antibody, or an anti-PD-1 antibody.
E24. The use of any one of embodiments E15 to E23, wherein the Bifidobacterium sp. is presented in FIG. 22.
E25. The use of any one of embodiments E15 to E24, wherein the Lactobacillus sp. is presented in FIG. 23.
E26. The use of any one of embodiments E15 to E25, where the Olsenella sp. is presented in FIG. 24.
E27. The use of any one of embodiments E15 to E26, further comprising a use of a chemotherapeutic agent, an immunotherapeutic agent, or a radiotherapy.
E28. The use of any one of embodiments E15 to E27, wherein said subject is a human.
E29. A kit for treating a subject having a cancer or suspected of having a cancer, comprising or consisting of, an immune checkpoint inhibitor and one or more bacterium selected from Bifidobacterium pseudolongum, Lactobacillus johnsonii, Olsenella profuse, Olsenella umbonata, or Olsenella uli, and optionally a container.
E30. A kit for treating a subject having a cancer or suspected of having a cancer, comprising or consisting of an immune checkpoint inhibitor and one or more bacterium selected from Bifidobacterium pseudolongum, Lactobacillus johnsonii, or Olsenella sp. and optionally a container.
E31. The kit of embodiment E29 or E30, where the cancer is colorectal cancer (CRC), lung cancer, melanoma, bladder cancer, kidney cancer, breast cancer, prostate cancer, stomach cancer, liver cancer, esophageal cancer, pancreatic cancer, brain cancer, cervical cancer, ovarian cancer, thyroid cancer, lip cancer, oral cancer, larynx cancer, nasopharynx cancer, or uterine cancer.
E32. The kit of embodiment E31, wherein the CRC is mismatch repair deficient (MMRD) CRC or inflammation-associated CRC.
E33. A kit for treating a subject having or suspected of having colorectal cancer (CRC), comprising or consisting of, administering an immune checkpoint inhibitor and one or more bacterium selected from Bifidobacterium pseudolongum, Lactobacillus johnsonii, Olsenella profuse, Olsenella umbonata, or Olsenella uli and optionally a container.
E34. A kit for treating a subject having or suspected of having colorectal cancer (CRC), comprising or consisting of, administering an immune checkpoint inhibitor and one or more bacteria selected from Bifidobacterium pseudolongum (B.p.), Lactobacillus johnsonii (L.j), or Olsenella sp. (O.sp.) and optionally a container.
E35. A kit for treating a subject having or suspected of having colorectal cancer (CRC), comprising or consisting of, administering an immune checkpoint inhibitor and one or more bacteria selected from Bifidobacterium sp. (B.sp.), Lactobacillus sp. (L.sp.), or Olsenella sp. (O.sp.) and optionally a container.
E36. The kit of any one of embodiments E33 to E35, wherein the CRC is mismatch repair deficient (MMRD) CRC or inflammation-associated CRC.
E37. The kit of any one of embodiments E29 to E36, wherein said ICB inhibitor is an anti-CTLA4 antibody, or an anti-PD-L1 antibody, or an anti-PD-1 antibody.
E38. The kit of any one of embodiments E29 to E37, wherein the Bifidobacterium sp. is presented in FIG. 22.
E39. The kit of any one of embodiments E29 to E38, wherein the Lactobacillus sp. is presented in FIG. 23.
E40. The kit of any one of embodiments E29 to E39, where the Olsenella sp. is presented in FIG. 24.
E41. The kit of any one of embodiments E29 to E40, further comprising administration of a chemotherapeutic agent, an immunotherapeutic agent, or a radiotherapy.
E42. The kit of any one of embodiments E29 to E41, wherein said subject is a human.
E42. A method of treating a subject having a cancer or suspected of having a cancer, comprising or consisting of, administering: an immune checkpoint inhibitor; inosine, a derivative of inosine, functional derivative of inosine, a prodrug of inosine, or a physiologically functional derivative of inosine; and a co-stimulant.
E43. The method of embodiment E42, where the cancer is colorectal cancer (CRC), lung cancer, melanoma, bladder cancer, kidney cancer, breast cancer, prostate cancer, stomach cancer, liver cancer, esophageal cancer, pancreatic cancer, brain cancer, cervical cancer, ovarian cancer, thyroid cancer, lip cancer, oral cancer, larynx cancer, nasopharynx cancer, or uterine cancer.
E44. The method of embodiment E43, wherein the CRC is mismatch repair deficient (MMRD) CRC or inflammation-associated CRC.
E45. A method of treating a subject having or suspected of having colorectal cancer (CRC), comprising or consisting of, administering: an immune checkpoint inhibitor; inosine, a derivative of inosine, functional derivative of inosine, a prodrug of inosine, or a physiologically functional derivative of inosine; and a co-stimulant.
E46. The method of embodiment E45, wherein the CRC is mismatch repair deficient (MMRD) CRC or inflammation-associated CRC.
E47. The method of any one of embodiments E42 to E46, wherein said ICB inhibitor is an anti-CTLA4 antibody, or an anti-PD-L1 antibody, or an anti-PD-1 antibody.
E48. The method of any one of embodiments E42 to E47, further comprising administration of a chemotherapeutic agent, an immunotherapeutic agent, or a radiotherapy.
E49. The method of any one of embodiments E42 to E48, where said co-stimulant is Toll like receptor (TLR) signals, CpG, LPS, Flagellin, Nucleotide-binding oligomerization domain-like receptors (NLRs), meso-diaminopimelic acid, muramyl dipeptide, ATP, extracellular glucose, crystals of monosodium urate, calcium pyrophosphate dihydrate, alum, cholesterol or environmental irritants; silica; asbestos; UV irradiation and skin irritants. RIG-I-like receptors (retinoic acid-inducible gene-I-like receptors), single- or double-stranded RNA (e.g., from viruses), C-type lectin receptors (CLR), repeated mannose units, C-type lectin domain, Cytokine receptor signalling, IL-12, IL-18, IL-33, IFN-g, Stimulation provided through antigen presenting cells or their counterpart on T-cells, CD80-CD28, CD86-CD28, CD40CD40L, OX-40L-OX40, -cGAS-STING pathway, for example, cytosolic DNA.
E50. The method of any one of embodiments E42 to E48, wherein said subject is a human.
E51. A Use of an immune checkpoint inhibitor; inosine, a derivative of inosine, functional derivative of inosine, a prodrug of inosine, or a physiologically functional derivative of inosine; and a co-stimulant, for treating a subject having a cancer or suspected of having a cancer.
E52. The use of embodiment E51, where the cancer is colorectal cancer (CRC), lung cancer, melanoma, bladder cancer, kidney cancer, breast cancer, prostate cancer, stomach cancer, liver cancer, esophageal cancer, pancreatic cancer, brain cancer, cervical cancer, ovarian cancer, thyroid cancer, lip cancer, oral cancer, larynx cancer, nasopharynx cancer, or uterine cancer.
E53. The use of embodiment E52, wherein the CRC is mismatch repair deficient (MMRD) CRC or inflammation-associated CRC.
E54. A use of an immune checkpoint inhibitor; inosine, a derivative of inosine, functional derivative of inosine, a prodrug of inosine, or a physiologically functional derivative of inosine; and a co-stimulant, for treating a subject having a cancer or suspected of having a cancer.
E55. The use of any one of embodiments E51 to E54, wherein said ICB inhibitor is an anti-CTLA4 antibody, or an anti-PD-L1 antibody, or an anti-PD-1 antibody.
E56. The use of any one of embodiments E51 to E55, further comprising use of a chemotherapeutic agent, an immunotherapeutic agent, or a radiotherapy.
E57. The use of any one of embodiments E51-E56, wherein the Toll like receptor (TLR) signals, CpG, LPS, Flagellin, Nucleotide-binding oligomerization domain-like receptors (NLRs), meso-diaminopimelic acid, muramyl dipeptide, ATP, extracellular glucose, crystals of monosodium urate, calcium pyrophosphate dihydrate, alum, cholesterol or environmental irritants; silica; asbestos; UV irradiation and skin irritants. RIG-I-like receptors (retinoic acid-inducible gene-I-like receptors), single- or double-stranded RNA (e.g., from viruses), C-type lectin receptors (CLR), repeated mannose units, C-type lectin domain, Cytokine receptor signalling, IL-12, IL-18, IL-33, IFN-g, Stimulation provided through antigen presenting cells or their counterpart on T-cells, CD80-CD28, CD86-CD28, CD40CD40L, OX-40L-OX40, -cGAS-STING pathway, for example, cytosolic DNA.
E58. The use of any one of embodiments E51 to E57, wherein said subject is a human.
E59. A kit for treating a subject having a cancer or suspected of having a cancer, comprising or consisting of an immune checkpoint inhibitor; inosine, a derivative of inosine, functional derivative of inosine, a prodrug of inosine, or a physiologically functional derivative of inosine; and a co-stimulant, and optionally a container.
E60. The kit of embodiment E59, where the cancer is colorectal cancer (CRC), lung cancer, melanoma, bladder cancer, kidney cancer, breast cancer, prostate cancer, stomach cancer, liver cancer, esophageal cancer, pancreatic cancer, brain cancer, cervical cancer, ovarian cancer, thyroid cancer, lip cancer, oral cancer, larynx cancer, nasopharynx cancer, or uterine cancer.
E61. The method of embodiment E60, wherein the CRC is mismatch repair deficient (MMRD) CRC or inflammation-associated CRC.
E62. The kit of any one of embodiments E59 to E61, wherein said ICB inhibitor is an anti-CTLA4 antibody, or an anti-PD-L1 antibody, or an anti-PD-1 antibody.
E63. The kit of any one of embodiments E59 to E62, further comprising a chemotherapeutic agent, an immunotherapeutic agent, or a radiotherapy.
E64. The kit of any one of embodiments E59 to E63, wherein the Toll like receptor (TLR) signals, CpG, LPS, Flagellin, Nucleotide-binding oligomerization domain-like receptors (NLRs), meso-diaminopimelic acid, muramyl dipeptide, ATP, extracellular glucose, crystals of monosodium urate, calcium pyrophosphate dihydrate, alum, cholesterol or environmental irritants; silica; asbestos; UV irradiation and skin irritants. RIG-I-like receptors (retinoic acid-inducible gene-I-like receptors), single- or double-stranded RNA (e.g., from viruses), C-type lectin receptors (CLR), repeated mannose units, C-type lectin domain, Cytokine receptor signalling, IL-12, IL-18, IL-33, IFN-g, Stimulation provided through antigen presenting cells or their counterpart on T-cells, CD80-CD28, CD86-CD28, CD40CD40L, OX-40L-OX40, -cGAS-STING pathway, for example, cytosolic DNA.
E65. The kit of any one of embodiments E59 to E64, wherein said subject is a human.

### Additional Exemplary Embodiments of the Invention

Additional Exemplary aspects of the invention are specified by the following embodiments.
A1. Use of one or more bacteria selected from Bifidobacterium sp. (B.sp.), Lactobacillus sp. (L.sp.), Olsenella sp. (O.sp.), or a combination thereof and an immune checkpoint inhibitor for treating a subject having a cancer or suspected of having a cancer.
A2. The use of embodiment A1, wherein the bacteria comprise one or more Bifidobacterium sp. presented in FIG. 22, Lactobacillus sp. presented in FIG. 23, and/or Olsenella sp. presented in FIG. 24, or a combination thereof.
A3. The use of any one of the foregoing embodiments, wherein said bacteria comprise a Bifidobacterium sp. comprising a 16S rDNA sequence having at least 85%, at least 90%, at least 95%, at least 96%, at least 97%, at least 98%, at least 99%, at least 99.5%, or having 100% identity to SEQ ID NO: 1.
A4. The use of any one of the foregoing embodiments, wherein said bacteria comprise a Lactobacillus sp. comprising a 16S rDNA sequence having at least 85%, at least 90%, at least 95%, at least 96%, at least 97%, at least 98%, at least 99%, at least 99.5%, or having 100% identity to SEQ ID NO: 2.
A5. The use of any one of the foregoing embodiments, wherein said bacteria comprise an Olsenella sp. comprising a 16S rDNA sequence having at least 85%, such as at least 90%, at least 95%, at least 96%, at least 97%, at least 98%, at least 99%, at least 99.5%, or having 100% identity to SEQ ID NO: 3.
A6. The use of any one of the foregoing embodiments, wherein said bacteria comprise Bifidobacterium pseudolongum, Lactobacillus johnsonii, Olsenella sp. or a combination thereof.
A7. The use of any one of the foregoing embodiments, wherein said bacteria comprise an Olsenella sp. comprising Olsenella profuse, Olsenella umbonata, or Olsenella uli, or a combination thereof.
A8. The use of any one of the foregoing embodiments, wherein said bacteria include the Bifidobacterium pseudolongum strain deposited as IDAC Deposit No. 231020-01.
A9. The use of any one of the foregoing embodiments, wherein said bacteria include the Lactobacillus johnsonii strain deposited as IDAC Deposit No. 231020-02.
A10. The use of any one of the foregoing embodiments, wherein said bacteria include the Olsenella sp. strain deposited as IDAC Deposit No. 231020-03.
A11. The use of any one of the foregoing embodiments, wherein said bacteria include at least two of the Bifidobacterium pseudolongum strain deposited as IDAC Deposit No. 231020-01, the Lactobacillus johnsonii strain deposited as IDAC Deposit No. 231020-02, and the Olsenella sp. strain deposited as IDAC Deposit No. 231020-03.
A12. The use of any one of the foregoing embodiments, wherein said bacteria produce elevated levels of inosine, xanthine, hypoxanthine, and/or inosine monophosphate, preferably inosine, in an in vitro or in vivo assay.
A13. The use of any one of the foregoing embodiments, wherein said bacteria produce elevated levels of inosine, xanthine, hypoxanthine, and/or inosine monophosphate, preferably inosine, when administered to said subject.
A14. The use of any one of the foregoing embodiments, wherein said bacteria are for administration to the gastrointestinal tract of said subject, preferably orally or rectally.
A15. Use of an immune checkpoint inhibitor and an additional agent selected from inosine, a derivative of inosine, functional derivative of inosine, a prodrug of inosine, or a physiologically functional derivative of inosine; and a co-stimulant for treating a subject having a cancer or suspected of having a cancer.
A16. The use of embodiment A15, wherein said additional agent comprises inosine, xanthine, hypoxanthine, inosine monophosphate, or a combination thereof.
A17. The use of embodiment A15, wherein said additional agent comprises an A2A agonist.
A18. The use of any one of embodiments A15 to A17, wherein said additional agent is for administration in an amount effective to potentiate the therapeutic effects of said ICB inhibitor on said cancer.
A19. The use of any one of embodiments A15 to A18, where said co-stimulant comprises one or more Toll like receptor (TLR) signals, CpG, LPS, Flagellin, Nucleotide-binding oligomerization domain-like receptors (NLRs), meso-diaminopimelic acid, muramyl dipeptide, ATP, extracellular glucose, crystals of monosodium urate, calcium pyrophosphate dihydrate, alum, cholesterol or environmental irritants; silica; asbestos; UV irradiation and skin irritants. RIG-I-like receptors (retinoic acid-inducible gene-I-like receptors), single- or double-stranded RNA (e.g., viral RNA), C-type lectin receptors (CLR), repeated mannose units, C-type lectin domain, cytokine receptor signalling, IL-12, IL-18, IL-33, IFN-g, stimulation provided through antigen presenting cells or their counterpart on T-cells, CD80-CD28, CD86-CD28, CD40CD40L, OX-40L-OX40, -cGAS-STING pathway, or cytosolic DNA.
A20. The use of any one of the foregoing embodiments, where the cancer is colorectal cancer (CRC), lung cancer, melanoma, bladder cancer, kidney cancer, breast cancer, prostate cancer, stomach cancer, liver cancer, esophageal cancer, pancreatic cancer, brain cancer, cervical cancer, ovarian cancer, thyroid cancer, lip cancer, oral cancer, larynx cancer, nasopharynx cancer, or uterine cancer, preferably CRC.
A21. The use of any one of embodiments A1-A19, wherein said cancer is selected from non-small cell lung cancer, small cell lung cancer, gastric carcinoma, testicular cancer, mesothelioma, head and neck cancers, glioblastoma, thymic carcinoma, or Merkel cell cancer. In another example, the cancer is selected from leukemias, myeloproliferative neoplasms (MPN), myelodysplastic syndromes (MDS), chronic lymphocytic leukemia (CLL), chronic myelocytic leukemia (CML), acute lymphoblastic leukemia (ALL), acute myeloid leukemia (ALL), myelodysplastic syndrome (MDS), Hodgkin lymphoma (HL), Non-Hodgkin lymphoma (NHL), multiple myeloma (MM), polycythemia vera (PV), essential thrombocythemia (ET), primary myelofibrosis (PMF), chronic eosinophilic leukemia, or mycosis fungoides.
A22. The use of any one of the foregoing embodiments, wherein said cancer is a mismatch repair deficient (MMRD) cancer or inflammation-associated cancer.
A23. The use of any one of the foregoing embodiments, wherein said cancer is a mismatch repair deficient (MMRD) colorectal cancer, gastrointestinal cancer, endometrial cancer, breast cancer, prostate cancer, bladder cancer, or thyroid cancer.
A24. The use of any one of the foregoing embodiments, wherein said cancer is a mismatch repair deficient (MMRD) cancer in a subject having a Lynch syndrome.
A25. The use of any one of the foregoing embodiments, wherein said cancer is mismatch repair deficient (MMRD) CRC or inflammation-associated CRC.
A26. The use of any one of embodiments A22 to A25, wherein said MMRD comprises (1) decreased or abolished expression of an MMRD protein selected from MLH1, MSH2, MSH6 and PMS2; and/or (2) methylation of an MMRD gene selected from MLH1, MSH2, MSH6 and PMS2, preferably MLH1; and/or (3) microsatellite instability.
A27. The use of any one of embodiments A22 to A26, wherein said use further comprises detecting MMRD in said cancer by a method comprising: (1) measuring expression of an MMRD protein selected from MLH1, MSH2, MSH6 and PMS2 in said cancer or a sample thereof, such as by immunohistochemical analysis; and/or (2) detecting methylation of an MMRD gene selected from MLH1, MSH2, MSH6 and PMS2, preferably MLH1 in said cancer or a sample thereof; and/or (3) detecting microsatellite instability in said cancer or a sample thereof.
A28. The use of any one of the foregoing embodiments, wherein said ICB inhibitor comprises an antagonist of CTLA-4, PD-1, PD-L1, PD-L2, LAG-3, VISTA, IDO, IDO1 IDO2, TIGIT, BTLA, HVEM, CD226 (DNAM-1), CD96 (Tactile), TIM-3, LAIR1, CD160 (BY55), CD244 (2B4), VTCN1 (B7-H4), KIR, A2_{A}R, B7-H3, or a combination thereof.
A29. The use of any one of the foregoing embodiments, wherein said ICB inhibitor comprises ipilimumab (YERVOY®, anti-CDLA-4 antibody, Bristol-Myers Squibb), nivolumab (OPDIVO®, anti-PD-1 antibody, Bristol-Myers Squibb), pembrolizumab (KEYTRUDA®, anti-PD-1 antibody, Merck), atezolizumab (TECENTRIQ®, anti-PD-L1 antibody, Roche), avelumab (BAVENCIO®, anti-PD-L1 antibody, Merck KGaA/Pfizer), durvalumab (IMFINZI®, anti-PD-L1 antibody, Medimmune/AstraZeneca), cemiplimab (LIBTAYOR, anti-PD-1 antibody, Regeneron/Sanofi), lambrolizumab (anti-PD-1 antibody, Merck), pidilizumab (anti-PD-1 and anti-DLL antibody, Medivation), BMS-936559 (anti-PD-L1, Bristol-Myers Squibb), MEDI-0680 (anti-PD-1 antibody; AMP-514; AstraZeneca), REGN2810 (anti-PD-1 antibody, Regeneron), CA-170 (small molecule PD-1 and PD-L1 inhibitor; Curis), BMS-1166 (small molecule PD-L1 inhibitor, Bristol-Myers Squibb), AMP-224 (anti-PD-1 fusion protein, Medimmune), spartalizumab (anti-PD-1 antibody, Novartis), STI-A1110 (anti-PD1 antibody, Sorrento/Servier), Dostarlimab (anti-PD-1 antibody, TSR-042, Tesaro), RG-7446 (anti-PD-L1 antibody, Roche), AUR-012 (peptide antagonist of PD1, Aurigene), STI-A1010 (anti-PD-L1 antibody, Sorrento), or a combination thereof.
A30. The use of any one of the foregoing embodiments, wherein said ICB inhibitor is an anti-CTLA4 antibody, or an anti-PD-L1 antibody, anti-PD-L2 antibody, or an anti-PD-1 antibody.
A31. The use of any one of the foregoing embodiments, wherein said use further comprises, prior to administration, measuring the level of inosine in the serum of said subject, wherein optionally said subject has reduced inosine levels prior to administration.
A32. The use of any one of the foregoing embodiments, wherein said use further comprises, prior to administration, measuring the level of said bacteria in the gastrointestinal tract of said subject, wherein optionally said subject has reduced or absent levels of said bacteria prior to administration.
A33. There use of any one of the foregoing embodiments, wherein said subject has completed a single dose of antibiotics or a course of antibiotics prior to, such as up to one day, two days, three days, four days, five days, six days, one week, up to two weeks, up to three weeks, or up to four weeks, prior to said administration.
A34. The use of any one of the foregoing embodiments, wherein said use further comprises administration of a chemotherapeutic agent, an immunotherapeutic agent, or a radiotherapy to said subject.
A35. The use of any one of the foregoing embodiments, wherein said subject is a human.
A36. The use of any one of the foregoing embodiments, wherein said bacteria are for administration in an amount comprising between 10⁶ and 10¹² colony forming units (CFU) of said bacteria, such as between 10⁷ and 10¹¹ CFU of said bacteria, between 10⁸ and 10¹¹ CFU of said bacteria, between 10⁹ and 10¹¹ CFU of said bacteria, or between 10⁹ and 10¹⁰ CFU of said bacteria.

What is claimed is:

1. A method of treating a subject having a cancer or suspected of having a cancer, comprising or consisting of, administering an effective amount of an immune checkpoint (ICB) inhibitor and one or more bacteria selected from Bifidobacterium sp. (B.sp.), Lactobacillus sp. (L.sp.), Olsenella sp. (O.sp.), or a combination thereof.

2. The method of claim 1, wherein:
(i) said bacteria comprise one or more Bifidobacterium sp. presented in FIG. 22, Lactobacillus sp. presented in FIG. 23, and/or Olsenella sp. presented in FIG. 24, or a combination thereof;
(ii) said bacteria of claim 1, or embodiment (i), comprises a Bifidobacterium sp. comprising a 16S rDNA sequence having at least 85%, at least 90%, at least 95%, at least 96%, at least 97%, at least 98%, at least 99%, at least 99.5%, or having 100% identity to SEQ ID NO: 1;
(iii) said bacteria of claim 1, or embodiment (i) or (ii), comprise a Lactobacillus sp. comprising a 16S rDNA sequence having at least 85%, at least 90%, at least 95%, at least 96%, at least 97%, at least 98%, at least 99%, at least 99.5%, or having 100% identity to SEQ ID NO: 2;
(iv) said bacteria of claim 1 or any one of embodiments (i) to (iii), comprise an Olsenella sp. comprising a 16S rDNA sequence having at least 85%, such as at least 90%, at least 95%, at least 96%, at least 97%, at least 98%, at least 99%, at least 99.5%, or having 100% identity to SEQ ID NO: 3;
(v) said bacteria of claim 1, or any one of embodiments (i) to (iv) comprise Bifidobacterium pseudolongum, Lactobacillus johnsonii, Olsenella sp. or a combination thereof;
(vi) said bacteria of claim 1, or any one of embodiments (i) to (v) comprise an Olsenella sp. comprising Olsenella profuse, Olsenella umbonata, or Olsenella uli, or a combination thereof.
(vii) said bacteria of claim 1, or any one of embodiments (i) to (vi) include the Bifidobacterium pseudolongum strain deposited as IDAC Deposit No. 231020-01;
(viii) said bacteria of claim 1, or any one of embodiments (i) to (vii) include the Lactobacillus johnsonii strain deposited as IDAC Deposit No. 231020-02;
(ix) said bacteria of claim 1, or any one of embodiments (i) to (viii) include the Olsenella sp. strain deposited as IDAC Deposit No. 231020-03;
(x) said bacteria of claim 1, or any one of embodiments (i) to (ix) include at least two of the Bifidobacterium pseudolongum strain deposited as IDAC Deposit No. 231020-01, the Lactobacillus johnsonii strain deposited as IDAC Deposit No. 231020-02, and the Olsenella sp. strain deposited as IDAC Deposit No. 231020-03;
(xi) said bacteria of claim 1 or any one of embodiments (i) to (x), produce elevated levels of inosine, xanthine, hypoxanthine, and/or inosine monophosphate, preferably inosine, in an in vitro or in vivo assay;
(xii) said bacteria of claim 1 or any one of embodiments (i) to (xi), produce elevated levels of inosine, xanthine, hypoxanthine, and/or inosine monophosphate, preferably inosine, in said subject; or
(xiii) said bacteria of claim 1 or any one of embodiments (i) to (xii), are administered to the gastrointestinal tract of said subject, preferably orally or rectally;
(xiv) said cancer of claim 1, any one of embodiments (i) to (xiii), is colorectal cancer (CRC), lung cancer, melanoma, bladder cancer, kidney cancer, breast cancer, prostate cancer, stomach cancer, liver cancer, esophageal cancer, pancreatic cancer, brain cancer, cervical cancer, ovarian cancer, thyroid cancer, lip cancer, oral cancer, larynx cancer, nasopharynx cancer, or uterine cancer, preferably CRC;
(xv) said cancer of claim 1, or any one of embodiments (i) to (xiv), is selected from non-small cell lung cancer, small cell lung cancer, gastric carcinoma, testicular cancer, mesothelioma, head and neck cancers, glioblastoma, thymic carcinoma, or Merkel cell cancer;
(xvi) said cancer of claim 1, or any one of embodiments (i) to (xv), is selected from leukemias, myeloproliferative neoplasms (MPN), myelodysplastic syndromes (MDS), chronic lymphocytic leukemia (CLL), chronic myelocytic leukemia (CML), acute lymphoblastic leukemia (ALL), acute myeloid leukemia (ALL), myelodysplastic syndrome (MDS), Hodgkin lymphoma (HL), Non-Hodgkin lymphoma (NHL), multiple myeloma (MM), polycythemia vera (PV), essential thrombocythemia (ET), primary myelofibrosis (PMF), chronic eosinophilic leukemia, or mycosis fungoides;
(xvii) said cancer of claim 1, or any one of embodiments (i) to (xvi), is a mismatch repair deficient (MMRD) cancer or inflammation-associated cancer, or has microsatellite instability (MSI);
(xviii) said cancer of claim 1, or any one of embodiments (i) to (xvii), is a mismatch repair deficient (MMRD) colorectal cancer, gastrointestinal cancer, endometrial cancer, breast cancer, prostate cancer, bladder cancer, or thyroid cancer;
(xix) said cancer of claim 1, or any one of embodiments (i) to (xviii), is a mismatch repair deficient (MMRD) cancer in a subject having a Lynch syndrome;
(xx) said cancer of claim 1, or any one of embodiments (i) to (xix), is mismatch repair deficient (MMRD) CRC or inflammation-associated CRC;
(xxi) said cancer of claim 1, or any one of embodiments (i) to (xi), is a mismatch repair deficient (MMRD) cancer, wherein said MMRD comprises (1) decreased or abolished expression of an MMRD protein selected from MLH1, MSH2, MSH6 and PMS2; and/or (2) methylation of an MMRD gene selected from MLH1, MSH2, MSH6 and PMS2, preferably MLH1; and/or (3) microsatellite instability;
(xxii) said method of claim 1, or any one of embodiments (i) to (xxi), further comprises detecting MMRD in said cancer by a method comprising: (1) measuring expression of an MMRD protein selected from MLH1, MSH2, MSH6 and PMS2 in said cancer or a sample thereof, such as by immunohistochemical analysis; and/or (2) detecting methylation of an MMRD gene selected from MLH1, MSH2, MSH6 and PMS2, preferably MLH1 in said cancer or a sample thereof; and/or (3) detecting microsatellite instability (MSI) in said cancer or a sample thereof;
(xxiii) said ICB inhibitor of claim 1, or any one of embodiments (i) to (xxii), comprises an antagonist of CTLA-4, PD-1, PD-L1, PD-L2, LAG-3, VISTA, IDO, ID01 ID02, TIGIT, BTLA, HVEM, CD226 (DNAM-1), CD96 (Tactile), TIM-3, LAIR1, CD160 (BY55), CD244 (2B4), VTCN1 (B7-H4), KIR, A2_{A}R, B7-H3, or a combination thereof;
(xxiv) said ICB inhibitor of claim 1, or any one of embodiments (i) to (xxiii), comprises ipilimumab (YERVOY®, anti-CDLA-4 antibody, Bristol-Myers Squibb), nivolumab (OPDIVO®, anti-PD-1 antibody, Bristol-Myers Squibb), pembrolizumab (KEYTRUDA®, anti-PD-1 antibody, Merck), atezolizumab (TECENTRIQ®, anti-PD-L1 antibody, Roche), avelumab (BAVENCIO®, anti-PD-L1 antibody, Merck KGaA/Pfizer), durvalumab (IMFINZI®, anti-PD-L1 antibody, Medimmune/AstraZeneca), cemiplimab (LIBTAYO®, anti-PD-1 antibody, Regeneron/Sanofi), lambrolizumab (anti-PD-1 antibody, Merck), pidilizumab (anti-PD-1 and anti-DLL antibody, Medivation), BMS-936559 (anti-PD-L1, Bristol-Myers Squibb), MEDI-0680 (anti-PD-1 antibody; AMP-514; AstraZeneca), REGN2810 (anti-PD-1 antibody, Regeneron), CA-170 (small molecule PD-1 and PD-L1 inhibitor; Curis), BMS-1166 (small molecule PD-L1 inhibitor, Bristol-Myers Squibb), AMP-224 (anti-PD-1 fusion protein, Medimmune), spartalizumab (anti-PD-1 antibody, Novartis), STI-A1110 (anti-PD1 antibody, Sorrento/Servier), Dostarlimab (anti-PD-1 antibody, TSR-042, Tesaro), RG-7446 (anti-PD-L1 antibody, Roche), AUR-012 (peptide antagonist of PD1, Aurigene), STI-A1010 (anti-PD-L1 antibody, Sorrento), or a combination thereof;
(xxv) said ICB inhibitor of claim 1, or any one of embodiments (i) to (xxiv), is an anti-CTLA4 antibody, or an anti-PD-L1 antibody, anti-PD-L2 antibody, or an anti-PD-1 antibody;
(xxvi) the method of claim 1, or any one of embodiments (i) to (xxv), further comprises, prior to said administration, measuring the level of inosine in the serum of said subject, wherein optionally said subject has reduced inosine levels prior to said administration;
(xxvii) the method of claim 1, or any one of embodiments (i) to (xxvi) further comprises, prior to said administration, measuring the level of said bacteria in the gastrointestinal tract of said subject, wherein optionally said subject has reduced or absent levels of said bacteria prior to said administration;
(xxviii) said subject of claim 1, or any one of embodiments (i) to (xxvii), has completed a single dose of antibiotics or a course of antibiotics prior to, such as up to one day, two days, three days, four days, five days, six days, one week, up to two weeks, up to three weeks, or up to four weeks, prior to said administration;
(xxix) the method of claim 1, or any one of embodiments (i) to (xxviii) further comprises the administration of a chemotherapeutic agent, an immunotherapeutic agent, or a radiotherapy to said subject;
(xxx) said subject of claim 1, or any one of embodiments (i) to (xxix), is a human;
(xxxi) said subject of claim 1, or any one of embodiments (i) to (xxx) is human and comprises an infant, child, adolescent, adult, or elderly person;
(xxxii) said subject claim 1, or any one of embodiments (i) to (xxxi), is a non-human animal, such as a non-human primate, a companion animal (e.g., a mammalian animal such as a dog, cat, ferret, horse, rabbit, guinea pig, gerbil, hamster, chinchilla, rat, mouse, or other small mammal; a bird; a reptile; a fish; an amphibian; an arthropod) or a livestock animal (e.g., a mammalian livestock animal such as a cow, pig, sheep, goat, alpaca, donkey, camel, water buffalo, or mink; or a chicken); or
(xxxiii) said bacteria claim 1, or any one of embodiments (i) to (xxxii) are administered to said subject in an amount comprising between 10⁵ and 10¹² colony forming units (CFU) of said bacteria, such as between 10⁷ and 10¹¹ CFU of said bacteria, between 10⁸ and 10¹¹ CFU of said bacteria, between 10⁹ and 10¹¹ CFU of said bacteria, or between 10⁹ and 10¹⁰ CFU of said bacteria.

3-14. (canceled)

15. A method of treating a subject having a cancer or suspected of having a cancer, comprising or consisting of, administering to said subject an effective amount of an immune checkpoint (ICB) inhibitor and an additional agent selected from inosine, a derivative of inosine, functional derivative of inosine, a prodrug of inosine, or a physiologically functional derivative of inosine; and optionally a co-stimulant.

16. The method of claim 15, wherein:
(i) said additional agent comprises inosine, xanthine, hypoxanthine, inosine monophosphate, or a combination thereof;
(ii) said additional agent of claim 15, or embodiment (i), comprises an A2A agonist;
(iii) said additional agent of claim 15, or embodiment (i) or (ii), is administered in an amount effective to potentiate the therapeutic effects of said ICB inhibitor on said cancer;
(iv) said co-stimulant of claim 15, or any one of embodiments (i) to (iii), comprises one or more Toll like receptor (TLR) signals, CpG, LPS, Flagellin, Nucleotide-binding oligomerization domain-like receptors (NLRs), meso-diaminopimelic acid, muramyl dipeptide, ATP, extracellular glucose, crystals of monosodium urate, calcium pyrophosphate dihydrate, alum, cholesterol or environmental irritants; silica; asbestos; UV irradiation and skin irritants. RIG-I-like receptors (retinoic acid-inducible gene-I-like receptors), single- or double-stranded RNA (e.g., viral RNA), C-type lectin receptors (CLR), repeated mannose units, C-type lectin domain, cytokine receptor signaling, IL-12, IL-18, IL-33, IFN-g, stimulation provided through antigen presenting cells or their counterpart on T-cells, CD80-CD28, CD86-CD28, CD40CD40L, OX-40L-OX40, -cGAS-STING pathway, or cytosolic DNA;
(v) said cancer of claim 15, any one of embodiments (i) to (iv), is colorectal cancer (CRC), lung cancer, melanoma, bladder cancer, kidney cancer, breast cancer, prostate cancer, stomach cancer, liver cancer, esophageal cancer, pancreatic cancer, brain cancer, cervical cancer, ovarian cancer, thyroid cancer, lip cancer, oral cancer, larynx cancer, nasopharynx cancer, or uterine cancer, preferably CRC;
(vi) said cancer of claim 15, or any one of embodiments (i) to (v), is selected from non-small cell lung cancer, small cell lung cancer, gastric carcinoma, testicular cancer, mesothelioma, head and neck cancers, glioblastoma, thymic carcinoma, or Merkel cell cancer;
(vii) said cancer of claim 15, or any one of embodiments (i) to (vi), is selected from leukemias, myeloproliferative neoplasms (MPN), myelodysplastic syndromes (MDS), chronic lymphocytic leukemia (CLL), chronic myelocytic leukemia (CML), acute lymphoblastic leukemia (ALL), acute myeloid leukemia (ALL), myelodysplastic syndrome (MDS), Hodgkin lymphoma (HL), Non-Hodgkin lymphoma (NHL), multiple myeloma (MM), polycythemia vera (PV), essential thrombocythemia (ET), primary myelofibrosis (PMF), chronic eosinophilic leukemia, or mycosis fungoides;
(viii) said cancer of claim 15, or any one of embodiments (i) to (vii) is a mismatch repair deficient (MMRD) cancer or inflammation-associated cancer, or has microsatellite instability (MSI);
(ix) said cancer of claim 15, or any one of embodiments (i) to (viii), is a mismatch repair deficient (MMRD) colorectal cancer, gastrointestinal cancer, endometrial cancer, breast cancer, prostate cancer, bladder cancer, or thyroid cancer;
(x) said cancer of claim 15, or any one of embodiments (i) to (ix), is a mismatch repair deficient (MMRD) cancer in a subject having a Lynch syndrome;
(xi) said cancer of claim 15, or any one of embodiments (i) to (x), is mismatch repair deficient (MMRD) CRC or inflammation-associated CRC;
(xii) said cancer of claim 15, or any one of embodiments (i) to (xi), is a mismatch repair deficient (MMRD) cancer, wherein said MMRD comprises (1) decreased or abolished expression of an MMRD protein selected from MLH1, MSH2, MSH6 and PMS2; and/or (2) methylation of an MMRD gene selected from MLH1, MSH2, MSH6 and PMS2, preferably MLH1; and/or (3) microsatellite instability;
(xiii) said method of claim 15, or any one of embodiments (i) to (xii), further comprises detecting MMRD in said cancer by a method comprising: (1) measuring expression of an MMRD protein selected from MLH1, MSH2, MSH6 and PMS2 in said cancer or a sample thereof, such as by immunohistochemical analysis; and/or (2) detecting methylation of an MMRD gene selected from MLH1, MSH2, MSH6 and PMS2, preferably MLH1 in said cancer or a sample thereof; and/or (3) detecting microsatellite instability (MSI) in said cancer or a sample thereof;
(xiv) said ICB inhibitor of claim 15, or any one of embodiments (i) to (xiii), comprises an antagonist of CTLA-4, PD-1, PD-L1, PD-L2, LAG-3, VISTA, IDO, ID01 ID02, TIGIT, BTLA, HVEM, CD226 (DNAM-1), CD96 (Tactile), TIM-3, LAIR1, CD160 (BY55), CD244 (2B4), VTCN1 (B7-H4), KIR, A2_{A}R, B7-H3, or a combination thereof;
(xv) said ICB inhibitor of claim 15, or any one of embodiments (i) to (xiv), comprises ipilimumab (YERVOY®, anti-CDLA-4 antibody, Bristol-Myers Squibb), nivolumab (OPDIVO®, anti-PD-1 antibody, Bristol-Myers Squibb), pembrolizumab (KEYTRUDA®, anti-PD-1 antibody, Merck), atezolizumab (TECENTRIQ®, anti-PD-L1 antibody, Roche), avelumab (BAVENCIO®, anti-PD-L1 antibody, Merck KGaA/Pfizer), durvalumab (IMFINZI®, anti-PD-L1 antibody, Medimmune/AstraZeneca), cemiplimab (LIBTAYO®, anti-PD-1 antibody, Regeneron/Sanofi), lambrolizumab (anti-PD-1 antibody, Merck), pidilizumab (anti-PD-1 and anti-DLL antibody, Medivation), BMS-936559 (anti-PD-L1, Bristol-Myers Squibb), MEDI-0680 (anti-PD-1 antibody; AMP-514; AstraZeneca), REGN2810 (anti-PD-1 antibody, Regeneron), CA-170 (small molecule PD-1 and PD-L1 inhibitor; Curis), BMS-1166 (small molecule PD-L1 inhibitor, Bristol-Myers Squibb), AMP-224 (anti-PD-1 fusion protein, Medimmune), spartalizumab (anti-PD-1 antibody, Novartis), STI-A1110 (anti-PD1 antibody, Sorrento/Servier), Dostarlimab (anti-PD-1 antibody, TSR-042, Tesaro), RG-7446 (anti-PD-L1 antibody, Roche), AUR-012 (peptide antagonist of PD1, Aurigene), STI-A1010 (anti-PD-L1 antibody, Sorrento), or a combination thereof;
(xvi) said ICB inhibitor of claim 15, or any one of embodiments (i) to (xv), is an anti-CTLA4 antibody, or an anti-PD-L1 antibody, anti-PD-L2 antibody, or an anti-PD-1 antibody;
(xvii) the method of claim 15, or any one of embodiments (i) to (xvi), further comprises, prior to said administration, measuring the level of inosine in the serum of said subject, wherein optionally said subject has reduced inosine levels prior to said administration;
(xviii) the method of claim 15, or any one of embodiments (i) to (xvii), further comprises, prior to said administration, measuring the level of said bacteria in the gastrointestinal tract of said subject, wherein optionally said subject has reduced or absent levels of said bacteria prior to said administration;
(xix) said subject of claim 15, or any one of embodiments (i) to (xviii), has completed a single dose of antibiotics or a course of antibiotics prior to, such as up to one day, two days, three days, four days, five days, six days, one week, up to two weeks, up to three weeks, or up to four weeks, prior to said administration;
(xx) the method of claim 15, or any one of embodiments (i) to (xix), further comprises the administration of a chemotherapeutic agent, an immunotherapeutic agent, or a radiotherapy to said subject;
(xxi) said subject of claim 15, or any one of embodiments (i) to (xx), is a human;
(xxii) said subject of claim 15, or any one of embodiments (i) to (xxi), is human and comprises an infant, child, adolescent, adult, or elderly person;
(xxiii) said subject of claim 15, or any one of embodiments (i) to (xxii), is a non-human animal, such as a non-human primate, a companion animal (e.g., a mammalian animal such as a dog, cat, ferret, horse, rabbit, guinea pig, gerbil, hamster, chinchilla, rat, mouse, or other small mammal; a bird; a reptile; a fish; an amphibian; an arthropod) or a livestock animal (e.g., a mammalian livestock animal such as a cow, pig, sheep, goat, alpaca, donkey, camel, water buffalo, or mink; or a chicken); and
(xxiv) said bacteria claim 15, or any one of embodiments (i) to (xxiii), are administered to said subject in an amount comprising between 10⁵ and 10¹² colony forming units (CFU) of said bacteria, such as between 10⁷ and 10¹¹ CFU of said bacteria, between 10⁸ and 10¹¹ CFU of said bacteria, between 10⁹ and 10¹¹ CFU of said bacteria, or between 10⁹ and 10¹⁰ CFU of said bacteria.

17-38. (canceled)

39. A kit for treating a subject having a cancer or suspected of having a cancer, comprising or consisting of, an immune checkpoint inhibitor and one or more bacterium as specified in claim 1 and optionally a container.

40. A kit for treating a subject having a cancer or suspected of having a cancer, comprising or consisting of an immune checkpoint inhibitor, an additional agent, and a co stimulant as specified in claim 15 and optionally a container.

41. An isolated bacterium selected from the following:
(i) one comprising a 16S rDNA sequence having at least 85%, at least 90%, at least 95%, at least 96%, at least 97%, at least 98%, at least 99%, at least 99.5%, or having 100% identity to SEQ ID NO: 1, preferably having at least 99.5%, or having 100% identity to SEQ ID NO: 1;
(ii) one comprising a 16S rDNA sequence having at least 85%, at least 90%, at least 95%, at least 96%, at least 97%, at least 98%, at least 99%, at least 99.5%, or having 100% identity to SEQ ID NO: 2, preferably having at least 99.5%, or having 100% identity to SEQ ID NO: 2;
(iii) one comprising a 16S rDNA sequence having at least 85%, such as at least 90%, at least 95%, at least 96%, at least 97%, at least 98%, at least 99%, at least 99.5%, or having 100% identity to SEQ ID NO: 3, preferably having at least 95%, at least 96%, at least 97% at least 98%, at least 99%, at least 99.5%, or having 100% identity to SEQ ID NO: 3;
(iv) an isolated Bifidobacterium pseudolongum strain deposited as IDAC Deposit No. 231020-01;
(v) an isolated Lactobacillus johnsonii strain deposited as IDAC Deposit No. 231020-02; and
(vi) an isolated Olsenella sp. strain deposited as IDAC Deposit No. 231020-03.

42-46. (canceled)

47. A composition selected from the following:
(i) one comprising at least one bacterium according to claim 41, in an effective amount for the treatment of a cancer and optionally further comprising a pharmaceutically acceptable carrier;
(ii) one comprising at least one bacterium according to claim 41, and a pharmaceutically acceptable carrier;
(iii) one comprising at least one bacterium according to claim 41, which is formulated for use as a drug;
(iv) one comprising at least one bacterium according to claim 41, which is formulated for use as a medical food;
(v) one comprising a mixture of two or more strains of bacteria according to claim 41, and optionally further comprising a pharmaceutically acceptable carrier;
(vi) one comprising two or more strains of bacteria to according to claim 41, in an amount effective for the treatment of a cancer and optionally further comprising a pharmaceutically acceptable carrier;
(vii) one comprising at least one bacterium according to claim 41, which is a food, beverage, food supplement, probiotic, or nutraceutical, preferably is formulated for ingestion;
(viii) one comprising a mixture of two or more strains of bacteria according to claim 41, which is a food, beverage, food supplement, probiotic, or nutraceutical comprising a mixture of two or more strains of bacteria according to claim 41, which preferably is formulated for ingestion.
(ix) one comprising at least one bacterium according to claim 41, wherein said bacteria produce elevated levels of inosine, xanthine, hypoxanthine, and/or inosine monophosphate, preferably inosine, in an in vitro or in vivo assay;
(x) one comprising at least one bacterium according to claim 41, wherein said bacterium or composition is lyophilized;
(xi) one comprising at least one bacterium according to claim 41, wherein said composition is adapted for administration to a subject, preferably a human subject, optionally an infant, child, adolescent, adult, or elderly;
(xii) one comprising at least one bacterium according to claim 41, wherein said composition is adapted for administration to a non-human animal, such as a non-human primate, a companion animal (e.g., a mammalian animal such as a dog, cat, ferret, horse, rabbit, guinea pig, gerbil, hamster, chinchilla, rat, mouse, or other small mammal; a bird; a reptile; a fish; an amphibian; an arthropod) or a livestock animal (e.g., a mammalian livestock animal such as a cow, pig, sheep, goat, alpaca, donkey, camel, water buffalo, or mink; or a chicken);
(xiii) one comprising at least one bacterium according to claim 41, wherein said composition is adapted for use in treating a subject having a cancer or suspected of having a cancer, in combination with an effective amount of an immune checkpoint (ICB) inhibitor; and
(xiv) one comprising at least one bacterium according to claim 41, wherein said composition contains an effective amount of said bacteria for treating a subject having a cancer or suspected of having a cancer.

48-61. (canceled)

62. A method of treating a subject having a cancer or suspected of having a cancer, comprising or consisting of, administering an effective amount of an immune checkpoint (ICB) inhibitor and one or more bacteria selected from Bifidobacterium sp. (B.sp.), Lactobacillus sp. (L.sp.), Olsenella sp. (O.sp.), or a combination thereof, wherein method comprises administering a bacterium, composition, food, beverage, food supplement, probiotic, or nutraceutical according to claim 41.

63. A composition comprising at least one bacterium according to claim 41, in an effective amount for use in the treatment of a cancer and optionally further comprising a pharmaceutically acceptable carrier.

64. A composition comprising a mixture of two or more strains of bacteria according to claim 41, in an effective amount for use in the treatment of a cancer and optionally further comprising a pharmaceutically acceptable carrier.


SEQUENCE LISTING (3 sequences)

SEQ ID NO: 1 - DNA, 1393 bp, Bifidobacterium pseudolongum
ggatccctggcagcttgctgccggggtgagagtggcgaacgggtgagtaatgcgtgaccgacctgccccatgcaccggaatagctcctggaaacgggtggtaatgccggatgttccacatgagcgcatgcgagtgtgggaaaggctttttgcggcatgggatggggtcgcgtcctatcagcttgttggtggggtaacggcctaccaaggcgttgacgggtagccggcctgagagggcgaccggccacattgggactgagatacggcccagactcctacgggaggcagcagtggggaatattgcacaatgggcgcaagcctgatgcagcgacgccgcgtgcgggatggaggccttcgggttgtaaaccgcttttgttcaagggcaaggcaggctttcgggcctgttgagtggattgttcgaataagcaccggctaactacgtgccagcagccgcggtaatacgtagggtgcaagcgttatccggatttattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggccctttttccgggtcctgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgtgccggacgcccgcggagacgcgggttcccttcggggccggttcacaggtggtgcatggtcgtcgtcagctcgtgtcgtgagatgttgggttaagtcccgcaacgagcgcaaccctcgccgcgtgttgccagcgggtcgtgccgggaactcacgtgggaccgccggggttaactcggaggaaggtggggatgacgtcagatcatcatgccccttacgtccagggcttcacgcatgctacaatggccggtacaacggggtgcgacacggtgacgtggggcggatccctgaaaaccggtctcagttcggatcgcagtctgcaactcgactgcgtgaaggtggagtcgctagtaatcgcggatcagcaacgccgcggtgaatgcgttcccgggccttgtacacaccgcccgtcaagtcatgaaagtgggcagcacccgaagacggtggcctaacccttgtggggggagccg

SEQ ID NO: 2 - DNA, 1436 bp, Lactobacillus johnsonii
  misc_feature (165)..(165): n is a, c, g, or t
  misc_feature (1410)..(1410): n is a, c, g, or t
gtcgagcgagcttgcctagatgattttagtgcttgcactaaatgaaactagatacaagcgagcggcggacgggtgagtaacacgtgggtaacctgcccaagagactgggataacacctggaaacagatgctaataccggataacaacactagacgcatgtctagngtttgaaagatggttctgctatcactcttggatggacctgcggtgcattagctagttggtaaggtaacggcttaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtaggtggccagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccagtgcaaacctaagagattaggtgttcccttcggggacgctgagacaggtggtgcatggctgtcgtcagctcgtgtcgtgagatgttgggttaagtcccgcaacgagcgcaacccttgtcattagttgccatcattaagttgggcactctaatgagactgccggtgacaaaccggaggaaggtggggatgacgtcaagtcatcatgccccttatgacctgggctacacacgtgctacaatggacggtacaacgagaagcgaacctgcgaaggcaagcggatctcttaaagccgttctcagttcggactgtaggctgcaactcgcctacacgaagctggaatcgctagtaatcgcggatcagcacgccgcggtgaatacgttcccgggccttgtacacaccgcccgtcacaccatgagagtctgtaacacccaaagccggtntggataacctttataggagtcagccg

SEQ ID NO: 3 - DNA, 1286 bp, Olsenella sp.
  misc_feature (5)..(5): n is a, c, g, or t
  misc_feature (38)..(38): n is a, c, g, or t
cgtantcctggacccgccgagagtggcgaacggctgantaacacgtgggcgacctgccccctccaccgggacagcctcgggaaaccgtgggtaataccggatactccgggacggccgcatggccggcccgggaaagcccagacgggaggggatgggcccgcggcctgttagctagtcggcggggtaacggcccaccgaggcgattatgggtagccgggttgagagaccgaccggccagattgggactgagacacggcccagactcctacgggaggcagcagtggggaatcttgcgcaatgggcggaagcctgacgcagcgacgccgcgtgcgggacgaaggccttcgggtcgtaaaccgctttcagcagggacgaggccctcacggtgacggtacctgcagaagaagccccggctaactacgtgccagcagccgcggtaatacgtagggggcgagcgttatccggattcattgggcgtaaagcgcgcgtaggcggcctgttaggtcgggggtcaaataccggggctcaaccccggtccgcccccgataccggcaggcttgagtctggtaggggaaggcggaattcccagtgtagcggtggaatgcgcagatattgggaagaacaccggcggcgaaggcggccttctgggccacgactgacgctgaggcgcgaaagctaggggagcgaacaggattagataccctggtagtcctagccgtaaacgatggacgctaggtgtggggggacctgccctccgtgccgcagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatcaccgtgaagcggcggagacgccgtggccgagaggagcggtgacaggtggtgcatggctgtcgtcagctcgtgtcgtgagatgttgggttaagtcccgcaacgagcgcaacccctgccgcgtgttgccagcattcagttgggcacccgcgcgggaccgccggcgtcaagccggaggagggtggggacgacgtcaagtcatcatgccccttatgtcctgggctgcacacgtgctacaatggccggcacaacgggctgcgaccgcgcgagcggaagcgaatccctcaaagccggccccagttcggatcggaggctgcaacccgcctccgtgaagtcggagttgctagtaatcgcggatcagcacgccgcggtgaatgcgttcccggg